(19) United States
(12) Patent Application Publication
(10) Pub. No.: US 2023/0303577 A1
(43) Pub. Date: Sep. 28, 2023

(54) TEMOZOLOMIDE ANALOGS AND METHODS OF USE

(71) Applicant: St. John's Cancer Institute, Santa Monica, CA (US)

(72) Inventors: Venkata Yenugonda, Woodland Hills, CA (US); Santosh Kesari, Santa Monica, CA (US)

(73) Assignee: St. John's Cancer Institute, Santa Monica, CA (US)

(21) Appl. No.: 18/018,435

(22) PCT Filed: Jul. 29, 2021
(86) PCT No.: PCT/US21/43773

§ 371 Date: Jan. 27, 2023

Related U.S. Application Data
(60) Provisional application No. 63/059,034, filed on Jul. 30, 2020.

(51) Int. Cl. C07D 487/04; A61P 35/00

(52) U.S. Cl. CPC C07D 487/04; A61P 35/00

(57) ABSTRACT

Disclosed herein are compounds and methods for treating cancer, such as glioblastomas.

### CROSS-REFERENCE TO RELATED APPLICATION

This application claims priority to U.S. Provisional Application No. 63/059,034 filed Jul. 30, 2020, which is incorporated by reference herein in its entirety.

### FIELD

The present disclosure is directed to embodiments of a temozolomide-based compound and method embodiments for using the same to treat cancer.

### BACKGROUND

Glioblastoma multiforme (GBM) is the most common and deadly primary brain tumor in adults. GBM carries a very poor prognosis, resulting in an ˜30% survival rate over 1 year, with less than 5% of patients surviving beyond 5 years. The median survival rate remains at ˜8 months. There are few effective treatment options for patients. The standard of care is surgical resection, followed by radiotherapy and concomitant oral chemotherapy using the DNA-alkylating agent temozolomide (TMZ). Unfortunately, residual tumor cells are invariably left behind following surgery. The residual, invasive tumor cells contribute to an almost universal tumor recurrence.

The antitumor effects of TMZ are mediated through methylation of the O⁶ position of guanine residues, with subsequent mismatch repair-dependent cell death. As a result, TMZ provides little benefit to patients with tumors that express O⁶-methylguanine DNA methyltransferase (MGMT), which makes the cells resistant to the effects of TMZ. Thus, patients with hypomethylated MGMT promoter do not respond to TMZ treatment. Although the lipophilic nature of TMZ allows it to cross the blood-brain barrier (BBB), TMZ retention in the cerebral parenchyma remains low at only 20%. Dose escalation of TMZ, to increase brain concentration and mitigate MGMT upregulation is limited by systemic toxicity, such as myelosuppression.

### SUMMARY

There exists a need in the art for compounds that exhibit increased brain retention and/or improved safety profiles to improve treatment outcomes. Disclosed herein are compound embodiments, particularly TMZ analogs and/or derivatives, with at least one improved property compared to TMZ.

In some embodiments, the compound can have a structure according to Formula I, including any pharmaceutically acceptable salt, prodrug, hydrate, solvate, or tautomer thereof

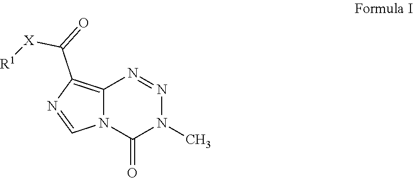

wherein

X can be oxygen or NR², wherein R² can be hydrogen, aliphatic, heteroaliphatic, haloaliphatic, haloheteroaliphatic, aromatic, or an organic functional group; and

R¹ can be aliphatic, heteroaliphatic, haloaliphatic, haloheteroaliphatic, aromatic, an organic functional group, or a biologically active compound. In some independent embodiments, if X is NR² and R² is hydrogen, then R¹ is not methyl, sec-butyl, iso-butyl, n-butyl, isopropyl, t-butyl, —(CH₂)₂-8-methyl-2,3,4,5-tetrahydro-1H-pyrido[4,3-b]indole, —(CH₂)₂-2,8-dimethyl-2,3,4,5-tetrahydro-1H-pyrido[4,3-b]indole, —(CH₂)₂-5,8-dimethyl-2,3,4,5-tetrahydro-1H-pyrido[4,3-b]indole, or —(CH₂)₆—N(H)-aminobenzoic acid. In yet another independent embodiment, if X is O, and R¹ is an aliphatic group, then the aliphatic group is other than methyl, ethyl, n-propyl, n-butyl, n-hexyl, n-heptyl, n-octyl, or methyl substituted with an aromatic group.

In some embodiments, R¹ can be aliphatic, heteroaliphatic, aryl, or heteroaryl.

In any or all of the above embodiments, X can be NR² wherein R² is hydrogen.

In any or all of the above embodiments, the compound can have a structure according to Formula II, including any pharmaceutically acceptable salt, prodrug, hydrate, solvate, or tautomer thereof

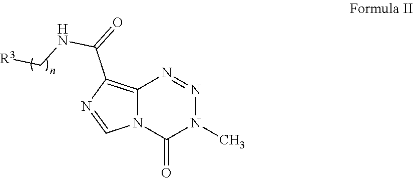

wherein R³ can be CH₃, ester, amide, alkoxy, or amine; and n can be an integer ranging from 1 to 10.

In some such embodiments, R³ can be CH₃, —OC(O)aliphatic, —N(H)C(O)aliphatic, —O-aliphatic, —N(aliphatic)₂, or —NHaliphatic; and n is an integer selected from 1 to 5.

In any or all of the above embodiments, R³ can be CH₃, —OC(O)Me, —N(H)C(O)Me, —OtBu, —N(Me)₂, or —NHEt; and n is 5.

In any or all of the above embodiments, the compound can have a structure according to Formula III, including any pharmaceutically acceptable salt, prodrug, hydrate, solvate, or tautomer thereof

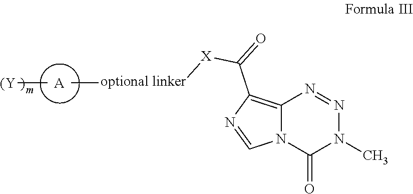

wherein ring A can be an aliphatic ring system, a heteroaliphatic ring system, or an aromatic ring system; the optional linker can be present or not and, if present, is an aliphatic group; each Y independently can be halogen, haloalkyl, alkyl, ester, or sulfonamide; and m can be an integer selected from 0 to 10.

In some embodiments, ring A can be a three-, four-, five-, six-, seven-, eight-, nine-, ten-, eleven-, or twelve-membered aliphatic ring system.

In any or all of the above embodiments, the aliphatic ring system can be cyclobutyl, cyclopentyl, cyclohexyl, bicyclopentyl, bicyclohexyl, bicycloheptenyl, or adamantyl.

In any or all of the above embodiments, each Y independently can be Cl, F, Br, I, CH₃, CF₃, —OC(O)CH₃, or —SO₂NH₂.

In any or all of the above embodiments, m can be 0, 1, or 2.

In any or all of the above embodiments, the compound can have a structure according to Formula IIIA′ or Formula IIIA″, including any pharmaceutically acceptable salt, prodrug, hydrate, solvate, or tautomer thereof,

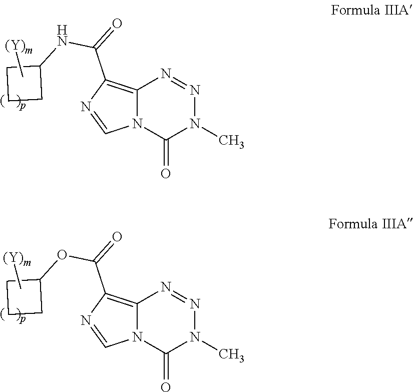

wherein p can be an integer selected from 0 to 5.

In any or all of the above embodiments, the compound can have a structure according to Formula IIIB, including any pharmaceutically acceptable salt, prodrug, hydrate, solvate, or tautomer thereof,

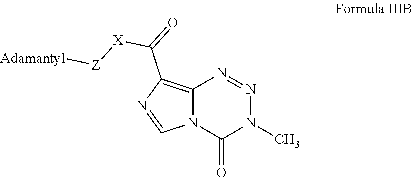

wherein X can be oxygen or NH; and Z can be bond or an aliphatic group.

In some embodiments, Z can be an aliphatic group and the aliphatic group can comprise one or more methylene groups, one or more cyclic groups, or a combination thereof and wherein the cyclic group is bound to the adamantyl or forms a spirocyclic group with the adamantyl.

In any or all of the above embodiments, the compound has a structure according to Formula IIIC, including any pharmaceutically acceptable salt, prodrug, hydrate, solvate, or tautomer thereof,

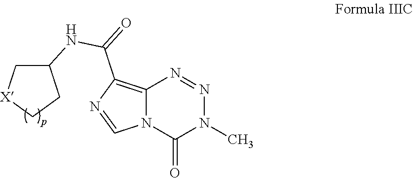

wherein X′ can be a selected from O, NH, or S and p can be an integer selected from 0 to 5.

In any or all of the above embodiments, the compound can have a structure according to Formula IIIC′ or IIIC″, including any pharmaceutically acceptable salt, prodrug, hydrate, solvate, or tautomer thereof,

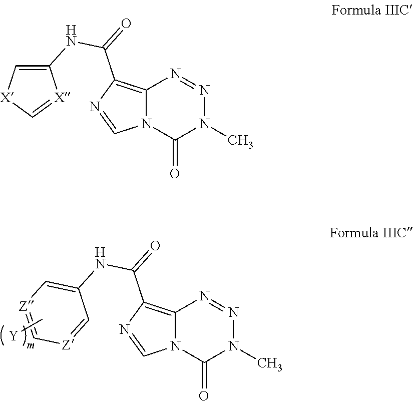

wherein X′ can be O, NH, S; X″ can be selected from O, N, S, or CH; and each of Z′ and Z″ independently can be CH, N, or an oxidized N atom.

In any or all of the above embodiments, the compound can have a structure according to Formulas IIID′ or IIID″, including any pharmaceutically acceptable salt, prodrug, hydrate, solvate, or tautomer thereof.

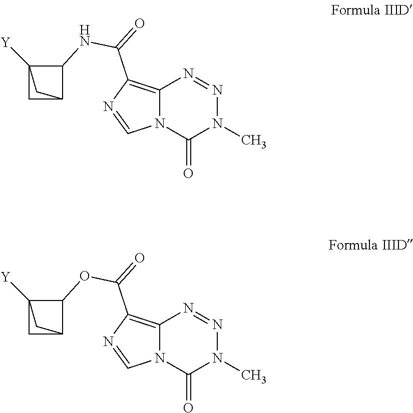

wherein Y is as recited above for any of Formulas III, IIIA′, or IIIA″.

In any or all of the above embodiments, the compound is selected from any of the compound species disclosed herein.

Also disclosed are embodiments of a composition comprising a compound according to any or all of the above embodiments and a pharmaceutically acceptable carrier.

Also disclosed herein are embodiments of a method of treating a subject with cancer, comprising administering to the subject an effective amount of a compound according to any or all of the above compound embodiments, or a pharmaceutical composition thereof to the subject. In some embodiments, the subject with cancer has glioblastoma, breast cancer, gastric cancer, colorectal cancer, head and neck cancer, melanoma, lung cancer, pancreatic cancer, bladder cancer, prostate cancer, or acute myeloblastic leukemia. In some such embodiments, the subject with cancer has glioblastoma multiforme. In any or all of the above embodiments, the subject with cancer has a tumor that expresses or overexpresses O⁶-methylguanine DNA methyltransferase (MGMT) and/or has hypomethylation or unmethylation of MGMT promoter.

In any or all of the above embodiments, the compound or pharmaceutical composition is administered orally or intravenously.

In any or all of the above embodiments, the method can further comprise administering to the subject one of more of surgery, radiation therapy, chemotherapy, or immunotherapy.

The foregoing and other objects and features of the present disclosure will become more apparent from the following detailed description, which proceeds with reference to the accompanying figures.

### BRIEF DESCRIPTION OF THE DRAWINGS

FIG. 1 is a graph showing cell proliferation of U87 cells in the presence of TMZ or the indicated compounds.

FIG. 2 is a graph showing 53BP1 foci per cell in U251 cells treated with TMZ or the indicated compounds.

FIG. 3 is a graph showing γH2AX per ell in U251 cells treated with TMZ or the indicated compounds.

FIG. 4 is a Western blot showing expression of MGMT in T98G cells following treatment with 100 μM O⁶-benzylguanine.

FIGS. 5A-5D are graphs showing aqueous stability of TMZ (FIG. 5A), TP3 (FIG. 5B), TP5 (FIG. 5C), and TP9 (FIG. 5D) at the indicated pHs.

FIGS. 6A and 6B are graphs showing relative brain:serum ratios of TMZ and its analogs measured 5 min into mice via intravenous route (FIG. 6A) and brain:serum ratios calculated based on test agent concentration in brain and serum, assuming a mouse blood volume of 60 mL/kg and brain weight of 0.5 g (FIG. 6B). Error is SEM, number of mice per cohort=3. Statistical significance compared to TMZ was determined by using a two-sample Student's t test (two-tailed test, assuming equal variance). Brain:serum ratio for each of the tested compounds was statistically significant compared to TMZ.

FIGS. 7A and 7B are graphs showing comparison of Cₘₐₓ brain to plasma ratio in oral dosing of the indicated compounds (FIG. 7A) and comparison of AUC brain to plasma ratio (FIG. 7B).

FIGS. 8A-8D are a series of graphs showing effect of TMZ (FIG. 8A) and TP3 (FIG. 8B) on cell viability of TMZ sensitive and resistant U87 cells and the effect of TMZ (FIG. 8C) and TP3 (FIG. 8D) on cell viability of TMZ sensitive and resistant U251 cells.

FIGS. 9A and 9B are graphs showing stability profile of Formulation 2 of TMZ (FIG. 9A) and TP3 (FIG. 9B), monitoring parent and AIC peak over 4 hours.

FIGS. 10A and 10B are graphs showing release kinetics profiles of TMZ and TP3 using Formulation 2 (FIG. 10A) or Formulation 3 (FIG. 10B).

FIGS. 11A-11D show results of treatment of a SCID mouse model of GBM with 20 mg/kg TMZ or TP3. Tumor volume (FIGS. 11A and 11B) and tumor weight (FIG. 11C) were determined in control (vehicle) and treated mice. Animal weight was monitored during the study (FIG. 11D).

FIG. 12 is a graph showing hydrolytic chemical stability of TP5-RGD over time.

FIG. 13 is a graph showing 53BP1 foci per 50 cells in U251 cells treated with TMZ or TP-RGD.

FIGS. 14A-14C is a series of panels showing anti-tumor activity of TMZ and TP9 in an orthotopic U87-Luc human glioblastoma tumor xenograft model. FIG. 14A shows effect of treatment on tumor burden for intracranial tumors as assessed by bioluminescence imaging (BLI) signaling on day 22. The day 22 mean BLI value (photon/s) was Control: 2.43E+08; TMZ: 1.87E+06 (P=0.02 TMZ vs. TP9); and TP9: 3.14E+07 (P=0.15 TP9 vs. Control). Statistical analysis was performed using a Student's t-test. FIG. 14B shows effect of treatment on body weight. Variability in average body weight after Day 23 was due to a decrease in the group size as individual mice reach peri morbidity endpoint. FIG. 14C shows neutrophil percentage on Day 27. Whole blood was collected on Day 27 from Vehicle, TMZ and TP9 (n=3/group) via submandibular survival bleed and CBC analysis was performed at TD2.

### DETAILED DESCRIPTION

Provided herein are substituted TMZ analogs with cell killing activity against multiple GBM cells lines with alkylation-mediated cell death. As described herein, the new compounds exhibit well-tolerated therapeutic doses, improved CNS physiochemical qualities, including brain vs. plasma retention (both IV and oral) and effective tumor growth inhibition in a GBM flank model. The compounds are also chemically stable, have moderate to high clearance rate, are not P-glycoprotein multidrug resistance protein substrates, and exhibit straightforward synthesis and formulations.

In specific embodiments, compounds TP3, TP5, and TP9 had improved CNS multiparameter optimization (MPO) score and log BB compared to TMZ, including greater brain-to-plasma ratio in both intravenous and oral administration routes. TP9 also demonstrated superior cell killing in unmethylated MGMT (MGMT-positive) cell lines and a greater DNA damage effect compared to TMZ. In addition, in an orthotopic GMB mouse model, TP9 was well tolerated in mice compared to TMZ, while showing similar growth inhibition. Thus, these compounds, and particularly TP9, are candidates for anti-cancer treatments, such as for GBM.

### I. Overview of Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood to one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

When chemical structures are depicted or described, unless explicitly stated otherwise, all carbons are assumed to include hydrogen so that each carbon conforms to a valence of four. For example, in the structure on the left-hand side of the schematic below there are nine hydrogen atoms implied. The nine hydrogen atoms are depicted in the right-hand structure.

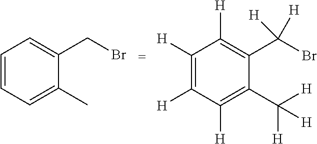

Sometimes a particular atom in a structure is described in textual formula as having a hydrogen or hydrogen atoms, for example —CH₂CH₂—. It will be understood by a person of ordinary skill in the art that the aforementioned descriptive techniques are common in the chemical arts to provide brevity and simplicity to description of organic structures.

If a group is depicted as “floating” on a ring system, as for example with Y in the structure illustrated below:

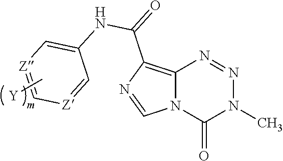

then, unless otherwise defined, the group (Y in the above structure) can reside on any atom of the fused bicyclic ring system, excluding the atom carrying the bond with the “ [symbol] ” symbol, so long as a stable structure is formed.

When a group R is depicted as existing on a ring system containing saturated carbons, as for example in the formula:

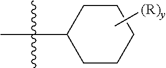

where, in this example, y can be more than one, assuming each replaces a currently depicted, implied, or expressly defined hydrogen on the ring; then, unless otherwise defined, two R's can reside on the same carbon. A simple example is when R is a methyl group. The depicted structure can exist as a geminal dimethyl on a carbon of the depicted ring (an “annular” carbon). In another example, two R's on the same carbon, including that same carbon, can be included in a ring, thus creating a spirocyclic ring (a “spirocyclyl” group) structure.

The term “substituted,” when used to modify a specified functional group and/or moiety, means that at least one, and perhaps two or more, hydrogen atoms of the specified functional group or moiety is independently replaced with one or more of the same or different substituent groups. In a particular embodiment, a functional group, moiety, or substituent may be substituted or unsubstituted, unless expressly defined as either “unsubstituted” or “substituted.” Accordingly, any of the functional groups specified herein may be unsubstituted or substituted unless the context indicates otherwise or a particular structural formula precludes substitution. In particular embodiments, a substituent may or may not be expressly defined as substituted, but is still contemplated to be optionally substituted. For example, an “aliphatic” or a “cyclic” moiety may be unsubstituted or substituted, but an “unsubstituted aliphatic” or an “unsubstituted cyclic” is not substituted.

The term “substituted” refers to all subsequent modifiers in a term, for example in the term “substituted arylaliphatic” substitution may occur on the “aliphatic” portion, the “aryl” portion or both. Additionally, in independent embodiments where a functional group or moiety is substituted with a substituted substituent, the nesting of such substituted substituents is limited to three, thereby preventing the formation of polymers. Thus, in such embodiments, in a functional group or moiety comprising a first group that is a substituent on a second group that is itself a substituent on a third group, which is attached to the parent structure, the first (outermost) group is only be substituted with unsubstituted substituents. For example, in a group comprising -(aryl-1)-(aryl-2)-(aryl-3), the “aryl-3” group is only be substituted with substituents that are not themselves substituted.

The above definitions and the following general formulas are not intended to include impermissible substitution patterns (e.g., methyl substituted with 5 fluoro groups). Such impermissible substitution patterns are easily recognized by a person having ordinary skill in the art.

A person of ordinary skill in the art will appreciate that compounds may exhibit the phenomena of tautomerism, conformational isomerism, geometric isomerism, and/or optical isomerism. For example, certain disclosed compounds can include one or more chiral centers and/or double bonds and as a consequence can exist as stereoisomers, such as double-bond isomers (e.g., geometric isomers), enantiomers, diastereomers, and mixtures thereof, such as racemic mixtures. As another example, certain disclosed compounds can exist in several tautomeric forms, including the enol form, the keto form, and mixtures thereof. As the various compound names, formulae and compound drawings within the specification and claims can represent only one of the possible tautomeric, conformational isomeric, optical isomeric, or geometric isomeric forms, a person of ordinary skill in the art will appreciate that the disclosed compounds encompass any tautomeric, conformational isomeric, optical isomeric, and/or geometric isomeric forms of the compounds described herein, as well as mixtures of these various different isomeric forms. Mixtures of different isomeric forms, including mixtures of enantiomers and/or stereoisomers, can be separated to provide each separate enantiomers and/or stereoisomer using techniques known to those of ordinary skill in the art, particularly with the benefit of the present disclosure. In cases of limited rotation, e.g. around the amide bond or between two directly attached rings such as pyridinyl rings, biphenyl groups, and the like, atropisomers are also possible and are also specifically included in the compounds of the present disclosure.

In any embodiments, any or all hydrogens present in the compound, or in a particular group or moiety within the compound, may be replaced by a deuterium or a tritium. Thus, a recitation of alkyl includes deuterated alkyl, where from one to the maximum number of hydrogens present may be replaced by deuterium. For example, ethyl refers to both C₂H₅ or C₂H₅ where from 1 to 5 hydrogens are replaced by deuterium, such as in C₂DₓH₅₋ₓ.

Acyl: —C(O)R^{a}, wherein R^{a} is selected from aliphatic, heteroaliphatic, haloaliphatic, haloheteroaliphatic, aromatic, or an organic functional group.

Acyl Halide: —C(O)X, wherein X is a halogen, such as Br, F, I, or Cl.

Aldehyde: —C(O)H.

Aliphatic: A hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl), alkenes (or alkenyl), alkynes (or alkynyl), including cyclic versions thereof, and further including straight- and branched-chain arrangements, and all stereo and position isomers as well.

Alkenyl: An unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cycloalkenyl), cis, or trans (e.g., E or Z).

Alkoxy: —O-aliphatic, such as —O-alkyl, —O-alkenyl, —O-alkynyl; with exemplary embodiments including, but not limited to, methoxy, ethoxy, n-propoxy, isopropoxy, n-butoxy, t-butoxy, sec-butoxy, n-pentoxy (wherein any of the aliphatic components of such groups can comprise no double or triple bonds, or can comprise one or more double and/or triple bonds).

Alkyl: A saturated monovalent hydrocarbon having at least one carbon atom to 50 carbon atoms (C₁₋₅₀), such as one to 25 carbon atoms (C₁₋₂₅), or one to ten carbon atoms (C₁₋₁₀), wherein the saturated monovalent hydrocarbon can be derived from removing one hydrogen atom from one carbon atom of a parent compound (e.g., alkane). An alkyl group can be branched, straight-chain, or cyclic (e.g., cycloalkyl).

Alkynyl: An unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkynyl).

Amide: —C(O)NR^{a}R^{b} or —NR^{a}C(O)R^{b} wherein each of R^{a} and R^{b} independently is selected from hydrogen, aliphatic, heteroaliphatic, haloaliphatic, haloheteroaliphatic, aromatic, or an organic functional group.

Amino: —NR^{a}R^{b}, wherein each of R^{a} and R^{b} independently is selected from hydrogen, aliphatic, heteroaliphatic, haloaliphatic, haloheteroaliphatic, aromatic, or an organic functional group.

Aromatic: A cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane π-electrons corresponds to the Hückel rule (4n+2). The point of attachment to the parent structure typically is through an aromatic portion of the condensed ring system. For example,

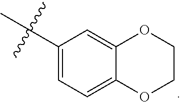 [SMILES: *c1ccc2c(c1)OCCO2]

However, in certain examples, context or express disclosure may indicate that the point of attachment is through a non-aromatic portion of the condensed ring system. For example,

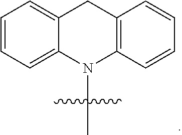 [SMILES: *N1c2ccccc2Cc2ccccc21]

An aromatic group or moiety may comprise only carbon atoms in the ring, such as in an aryl group or moiety, or it may comprise one or more ring carbon atoms and one or more ring heteroatoms comprising a lone pair of electrons (e.g. S, O, N, P, or Si), such as in a heteroaryl group or moiety.

Aromatic groups may be substituted with one or more groups other than hydrogen, such as aliphatic, heteroaliphatic, haloaliphatic, haloheteroaliphatic, aromatic, or an organic functional group.

Aryl: An aromatic carbocyclic group comprising at least five carbon atoms to 15 carbon atoms (C₅-C₁₅), such as five to ten carbon atoms (C₅-C₁₀), having a single ring or multiple condensed rings, which condensed rings can or may not be aromatic provided that the point of attachment to a remaining position of the compounds disclosed herein is through an atom of the aromatic carbocyclic group. Aryl groups may be substituted with one or more groups other than hydrogen, such as aliphatic, heteroaliphatic, haloaliphatic, haloheteroaliphatic, aromatic, or an organic functional group.

Aroxy: —O-aromatic.

Azo: —N═NR^{a} wherein R^{a} is hydrogen, aliphatic, heteroaliphatic, haloaliphatic, haloheteroaliphatic, aromatic, or an organic functional group.

Carbamate: —OC(O)NR^{a}R^{b}, wherein each of R^{a} and R^{b} independently is selected from hydrogen, aliphatic, heteroaliphatic, haloaliphatic, haloheteroaliphatic, aromatic, or an organic functional group.

Carboxyl: —C(O)OH.

Carboxylate: —C(O)O or salts thereof, wherein the negative charge of the carboxylate group may be balanced with an M⁺ counterion, wherein M⁺ may be an alkali ion, such as K⁺, Na⁺, Li⁺; an ammonium ion, such as *N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Carrier or Vehicle: An excipient that serves as a component capable of delivering a compound described herein. In some embodiments, a carrier can be a suspension aid, solubilizing aid, or aerosolization aid. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In some examples, the pharmaceutically acceptable carrier may be sterile to be suitable for administration to a subject (for example, by parenteral, intramuscular, or subcutaneous injection). In addition to biologically-neutral carriers, pharmaceutical formulations to be administered can contain minor amounts of non-toxic auxiliary substances, such as wetting or emulsifying agents, preservatives, and pH buffering agents and the like, for example sodium acetate or sorbitan monolaurate.

Cyano: —CN.

Disulfide: —SSR^{a}, wherein R^{a} is selected from hydrogen, aliphatic, heteroaliphatic, haloaliphatic, haloheteroaliphatic, aromatic, or an organic functional group.

Dithiocarboxylic: —C(S)SR^{a} wherein R^{a} is selected from hydrogen, aliphatic, heteroaliphatic, haloaliphatic, haloheteroaliphatic, aromatic, or an organic functional group.

Ester: —C(O)OR^{a} or —OC(O)R^{a}, wherein R^{a} is selected from aliphatic, heteroaliphatic, haloaliphatic, haloheteroaliphatic, aromatic, or an organic functional group.

Ether: -aliphatic-O-aliphatic, -aliphatic-O-aromatic, -aromatic-O-aliphatic, or -aromatic-O-aromatic.

Glioma: A type of central nervous system tumor arising from glial cells. Gliomas include astrocytomas, ependymomas, and oligodendrogliomas. In some examples, the glioma is glioblastoma multiforme (GBM) an aggressive and difficult to treat tumor.

Halo (or halide or halogen): Fluoro, chloro, bromo, or iodo.

Haloaliphatic: An aliphatic group wherein one or more hydrogen atoms, such as one to 10 hydrogen atoms, independently is replaced with a halogen atom, such as fluoro, bromo, chloro, or iodo.

Haloalkyl: An alkyl group wherein one or more hydrogen atoms, such as one to 10 hydrogen atoms, independently is replaced with a halogen atom, such as fluoro, bromo, chloro, or iodo. In an independent embodiment, haloalkyl can be a CX₃ group, wherein each X independently can be selected from fluoro, bromo, chloro, or iodo.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group. Alkoxy, ether, amino, disulfide, peroxy, and thioether groups are exemplary (but non-limiting) examples of heteroaliphatic. In some embodiments, a fluorophore can also be described herein as a heteroaliphatic group, such as when the heteroaliphatic group is a heterocyclic group.

Heteroaryl: An aryl group comprising at least one heteroatom to six heteroatoms, such as one to four heteroatoms, which can be selected from, but not limited to oxygen, nitrogen, sulfur, silicon, boron, selenium, phosphorous, and oxidized forms thereof within the ring. Such heteroaryl groups can have a single ring or multiple condensed rings, wherein the condensed rings may or may not be aromatic and/or contain a heteroatom, provided that the point of attachment is through an atom of the aromatic heteroaryl group. Heteroaryl groups may be substituted with one or more groups other than hydrogen, such as aliphatic, heteroaliphatic, haloaliphatic, haloheteroaliphatic, aromatic, or an organic functional group. In some embodiments, a fluorophore can also be described herein as a heteroaryl group.

Heteroatom: An atom other than carbon or hydrogen, such as (but not limited to) oxygen, nitrogen, sulfur, silicon, boron, selenium, or phosphorous. In particular disclosed embodiments, such as when valency constraints do not permit, a heteroatom does not include a halogen atom.

Organic Functional Group: A functional group that may be provided by any combination of aliphatic, heteroaliphatic, aromatic, haloaliphatic, and/or haloheteroaliphatic groups, or that may be selected from, but not limited to, aldehyde; aroxy; acyl halide; nitro; cyano; azide; carboxyl (or carboxylate); amide; acyl; carbonate; imine; azo; carbamate; hydroxyl; haloalkyl; thiol; sulfonyl (or sulfonate); oxime; ester; thiocyanate; thioacyl; thiocarboxylic acid; thioester; dithiocarboxylic acid or ester; phosphonate; phosphate; silyl ether; sulfinyl; sulfonamide; thial; or combinations thereof.

Oxime: —CR^{a}=NOH, wherein R^{a} is hydrogen, aliphatic, heteroaliphatic, haloaliphatic, haloheteroaliphatic, aromatic, or an organic functional group.

Peroxy: —O—OR^{a} wherein R^{a} is hydrogen, aliphatic, heteroaliphatic, haloaliphatic, haloheteroaliphatic, aromatic, or an organic functional group.

Pharmaceutically Acceptable Excipient: A substance, other than a compound that is included in a formulation of the compound. As used herein, an excipient may be incorporated within particles of a pharmaceutical composition, or it may be physically mixed with particles of a pharmaceutical composition. An excipient also can be in the form of a solution, suspension, emulsion, or the like. An excipient can be used, for example, to dilute an active agent and/or to modify properties of a pharmaceutical composition. Excipients can include, but are not limited to, antiadherents, binders, coatings, enteric coatings, disintegrants, flavorings, sweeteners, colorants, lubricants, glidants, sorbents, preservatives, adjuvants, carriers or vehicles. Excipients may be starches and modified starches, cellulose and cellulose derivatives, saccharides and their derivatives such as disaccharides, polysaccharides and sugar alcohols, protein, synthetic polymers, crosslinked polymers, antioxidants, amino acids or preservatives. Exemplary excipients include, but are not limited to, magnesium stearate, stearic acid, vegetable stearin, sucrose, lactose, starches, hydroxypropyl cellulose, hydroxypropyl methylcellulose, xylitol, sorbitol, maltitol, gelatin, polyvinylpyrrolidone (PVP), polyethylene glycol (PEG), propylene glycol, tocopheryl polyethylene glycol 1000 succinate (also known as vitamin E TPGS, or TPGS), carboxy methyl cellulose, dipalmitoyl phosphatidyl choline (DPPC), vitamin A, vitamin E, vitamin C, retinyl palmitate, selenium, cysteine, methionine, citric acid, sodium citrate, methyl paraben, propyl paraben, sugar, silica, talc, magnesium carbonate, sodium starch glycolate, tartrazine, aspartame, benzalkonium chloride, vegetable oil, propyl gallate, sodium metabisulfite, or lanolin. In independent embodiments, water is not intended as a pharmaceutically acceptable excipient.

Pharmaceutically Acceptable Salt: Pharmaceutically acceptable salts of a compound described herein that are derived from a variety of organic and inorganic counter ions as will be known to a person of ordinary skill in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. “Pharmaceutically acceptable acid addition salts” are a subset of “pharmaceutically acceptable salts” that retain the biological effectiveness of the free bases while formed by acid partners. In particular, the disclosed compound embodiments form salts with a variety of pharmaceutically acceptable acids, including, without limitation, inorganic acids such as hydrochloric acid, hydrobromic acid, sulfuric acid, nitric acid, phosphoric acid, and the like, as well as organic acids such as formic acid, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, benzene sulfonic acid, isethionic acid, methanesulfonic acid, ethanesulfonic acid, p-toluenesulfonic acid, salicylic acid, and the like. “Pharmaceutically acceptable base addition salts” are a subset of “pharmaceutically acceptable salts” that are derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. See, for example, S. M. Berge, et al., “Pharmaceutical Salts,” J. Pharm. Sci., 1977; 66:1-19 which is incorporated herein by reference.

Phosphate: —O—P(O)(OR^{a})₂, wherein each R^{a} independently is hydrogen, aliphatic, heteroaliphatic, haloaliphatic, haloheteroaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as *N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Phosphonate: —P(O)(OR^{a})₂, wherein each R^{a} independently is hydrogen, aliphatic, heteroaliphatic, haloaliphatic, haloheteroaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as *N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Prodrug: Compound embodiments disclosed herein that are transformed, most typically in vivo, to yield a biologically active compound, particularly the parent compound, for example, by hydrolysis in the gut or enzymatic conversion. Common examples of prodrug moieties include, but are not limited to, pharmaceutically acceptable ester and amide forms of a compound having an active form bearing a carboxylic acid moiety.

Examples of pharmaceutically acceptable esters of the compound embodiments of the present disclosure include, but are not limited to, esters of phosphate groups and carboxylic acids, such as aliphatic esters, particularly alkyl esters (for example C₁₋₆alkyl esters). Other prodrug moieties include phosphate esters, such as —CH₂—O—P(O)(OR^{a})₂ or a salt thereof, wherein R^{a} is hydrogen or aliphatic (e.g., C₁₋₆alkyl). Acceptable esters also include cycloalkyl esters and arylalkyl esters such as, but not limited to, benzyl. Examples of pharmaceutically acceptable amides of the compound embodiments of this disclosure include, but are not limited to, primary amides, and secondary and tertiary alkyl amides (for example with between one and six carbons). Amides and esters of disclosed exemplary embodiments of compound embodiments according to the present disclosure can be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference.

Silyl Ether: —OSiR^{a}R^{b}, wherein each of R^{a} and R^{b} independently is selected from hydrogen, aliphatic, heteroaliphatic, haloaliphatic, haloheteroaliphatic, aromatic, or an organic functional group.

Subject: Mammals and other animals, such as humans, companion animals (e.g., dogs, cats, rabbits, etc.), utility animals, and feed animals; thus, disclosed methods are applicable to both human therapy and veterinary applications.

Sulfinyl: —S(O)R^{a}, wherein R^{a} is selected from hydrogen, aliphatic, heteroaliphatic, haloaliphatic, haloheteroaliphatic, aromatic, or an organic functional group.

Sulfonyl: —SO₂R^{a}, wherein R^{a} is selected from hydrogen, aliphatic, heteroaliphatic, haloaliphatic, haloheteroaliphatic, aromatic, or an organic functional group.

Sulfonamide: —SO₂NR^{a}R^{b} or —N(R^{a})SO₂R^{b}, wherein each of R^{a} and R^{b} independently is selected from hydrogen, aliphatic, heteroaliphatic, haloaliphatic, haloheteroaliphatic, aromatic, or an organic functional group.

Sulfonate: —SO₃, wherein the negative charge of the sulfonate group may be balanced with an M⁺ counter ion, wherein M⁺ may be an alkali ion, such as K⁺, Na⁺, Li⁺; an ammonium ion, such as +N(R^{b})₄ where R^{b}is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Therapeutically Effective Amount: An amount of a compound sufficient to treat a specified disorder or disease, or to ameliorate or eradicate one or more of its symptoms and/or to inhibit occurrence or recurrence of the disease or disorder. The amount of a compound which constitutes a “therapeutically effective amount” will vary depending on the compound, the disease state and its severity, the age and condition of the patient to be treated, and the like. The therapeutically effective amount can be determined by a person of ordinary skill in the art.

Thial: —C(S)H.

Thioacyl: —C(S)R^{a} wherein R^{a} is selected from hydrogen, aliphatic, heteroaliphatic, haloaliphatic, haloheteroaliphatic, aromatic, or an organic functional group.

Thiocarboxylic acid: —C(O)SH, or —C(S)OH.

Thiocyanate: —S—CN or —N═C═S.

Thioester: —C(O)SR^{a} or —C(S)OR^{a} wherein R^{a} is selected from hydrogen, aliphatic, heteroaliphatic, haloaliphatic, haloheteroaliphatic, aromatic, or an organic functional group.

Thioether: —S-aliphatic or —S-aromatic, such as —S-alkyl, —S-alkenyl, —S-alkynyl, —S-aryl, or —S— heteroaryl; or -aliphatic-S-aliphatic, -aliphatic-S-aromatic, -aromatic-S-aliphatic, or -aromatic-S-aromatic.

Treating, Treatment, and Therapy: Any success or indicia of success in the attenuation or amelioration of an injury, pathology or condition, including any objective or subjective parameter such as abatement, remission, diminishing of symptoms or making the condition more tolerable to the patient, slowing in the rate of disease progression, or improving a subject's physical or mental well-being. The treatment may be assessed by objective or subjective parameters; including the results of a physical examination, neurological examination, and/or psychiatric evaluation.

As used herein, the terms “disease” and “condition” can be used interchangeably or can be different in that the particular malady or condition may not have a known causative agent (so that etiology has not yet been determined) and it is therefore not yet recognized as a disease but only as an undesirable condition or syndrome, where a more or less specific set of symptoms have been identified by clinicians.

### II. Compound Embodiments

Disclosed herein are embodiments of a compound that can be used in method and composition embodiments disclosed herein.

In particular embodiments, the compound has a structure according to Formula I, including any pharmaceutically acceptable salt, prodrug, hydrate, solvate, or tautomer thereof.

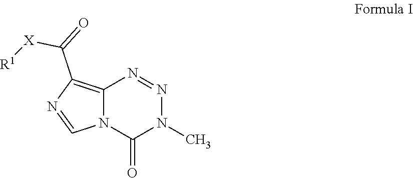

With reference to Formula I, X can be oxygen or NR² wherein R² is selected from hydrogen, aliphatic, heteroaliphatic, haloaliphatic, haloheteroaliphatic, aromatic, or an organic functional group; and R¹ is selected from aliphatic, heteroaliphatic, haloaliphatic, haloheteroaliphatic, aromatic, an organic functional group, or a biologically active compound. In particular embodiments, R¹ is selected from aliphatic, heteroaliphatic, aryl, or heteroaryl, which can be substituted or unsubstituted.

In embodiments where such groups are substituted, the substituents can be selected from aliphatic substituents, heteroaliphatic substituents, aromatic substituents, haloaliphatic substituents, haloheteroaliphatic substituents, and/or organic functional group substituents. In particular embodiments, X is NR² wherein R² is hydrogen. In some particular embodiments, if X is NR² wherein R² is hydrogen and R¹ is aliphatic, then the aliphatic group comprises at least 5 carbon atoms. In an independent embodiment, R² is not aliphatic. In another independent embodiment, if X is NR² and R² is hydrogen, then R¹ is not methyl, sec-butyl, iso-butyl, n-butyl, isopropyl, t-butyl, —(CH₂)₂-8-methyl-2,3,4,5-tetrahydro-1H-pyrido[4,3-b]indole (wherein the pyrido N atom is attached to the —(CH₂)₂— group), —(CH₂)₂-2,8-dimethyl-2,3,4,5-tetrahydro-1H-pyrido[4,3-b]indole (wherein the indole N atom is attached to the —(CH₂)₂— group), —(CH₂)₂-5,8-dimethyl-2,3,4,5-tetrahydro-1H-pyrido[4,3-b]indole (wherein the pyrido N atom is attached to the —(CH₂)₂— group), or —(CH₂)₆—N(H)-aminobenzoic acid. In particular embodiments, if X is O and R¹ is an aliphatic group, then the aliphatic group is selected from other than methyl, ethyl, n-propyl, n-butyl, n-hexyl, n-heptyl, n-octyl, or methyl substituted with an aromatic group. In particular embodiments wherein R¹ is a biologically active compound, it can be an αVβ3 integrin inhibitor, such as RGDyK (or cyclic RGDyK).

In some embodiments, the compound has a structure according to Formula II, including any pharmaceutically acceptable salt, prodrug, hydrate, solvate, or tautomer thereof.

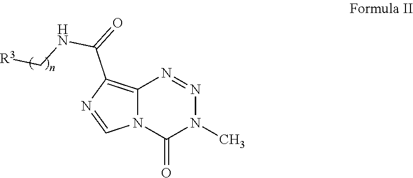

wherein R³ is CH₃, ester, amide, alkoxy, or amine; and n is an integer ranging from 1 to 10, such as 1 to 8, or 1 to 5, or 1, 2, 3, 4, 5, 6, 7, 8, 9, or 10. In particular embodiments, R³ is CH₃, —OC(O)aliphatic, —N(H)C(O)aliphatic, —O-aliphatic, —N(aliphatic)₂, or -NHaliphatic; and n is an integer selected from 1 to 5. In particular embodiments, R³ is CH₃, —OC(O)Me, —N(H)C(O)Me, -OtBu, —N(Me)₂, or -NHEt; and n is 5.

In some embodiments, the compound has a structure according to Formula III, including any pharmaceutically acceptable salt, prodrug, hydrate, solvate, or tautomer thereof,

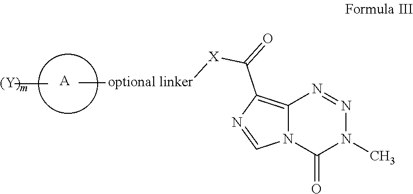

wherein X is as recited above for Formula I, ring A is an aliphatic ring system, a heteroaliphatic ring system, or an aromatic ring system; the optional linker group is an aliphatic group and may or may not be present, and each Y independently is halogen, haloalkyl, alkyl, ester, or sulfonamide; and m is an integer selected from 0 to 10, such as 0 to 8, or 0 to 5, or 1 to 10 or 1 to 8 or 1 to 5, or 0, 1, 2, 3, 4, 5, 6, 7, 8, 9, or 10. The aliphatic, heteroaliphatic, and/or aromatic ring systems can be substituted or unsubstituted. In some embodiments comprising substituents, the substituents can comprise aliphatic groups, halogen atoms, haloalkyl groups, ester groups, or sulfonamide groups. In particular embodiments, each Y independently is selected from Cl, F, Br, or I; CH₃; CF₃; —OC(O)CH₃; or —SO₂NH₂. In some embodiments, one or two Y groups are present and in embodiments where two Y groups are present, each Y can be the same or different.

In particular embodiments, ring A is a three-, four-, five-, six-, seven-, eight-, nine-, ten-, eleven-, or twelve-membered aliphatic ring system, including monocyclic or multicyclic ring systems, wherein any multicyclic ring systems can comprise fused, spirocyclic, and/or bridged cyclic ring systems. In particular embodiments, ring A is an aliphatic ring system selected from cyclobutyl, cyclopentyl, cyclohexyl, bicyclopentyl, bicyclohexyl, bicycloheptenyl, or adamantyl. In some embodiments, a combination of such groups can be present, such as wherein ring A is a cyclopentyl group comprising an adamantyl substituent. In some embodiments, the aliphatic ring system can comprise one or more Y substituents, which can be bound at any position on the aliphatic ring system. In some embodiments, the compound can have a structure according to Formula IIIA′ or Formula IIIA″, including any pharmaceutically acceptable salt, prodrug, hydrate, solvate, or tautomer thereof,

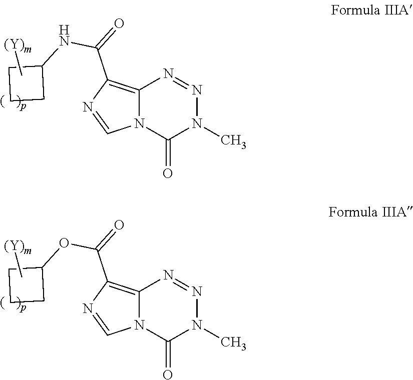

wherein each Y and m are as recited for Formula III and p is an integer selected from 0 to 5, such as 0 to 4, or 0 to 3, or 0 to 2, or 0, 1, 2, 3, 4, or 5.

In some embodiments, the compound can have a structure according to Formula IIIB, including any pharmaceutically acceptable salt, prodrug, hydrate, solvate, or tautomer thereof,

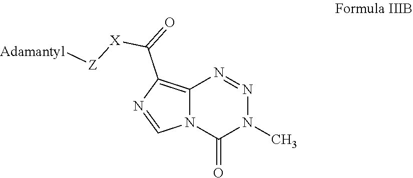

wherein X can be oxygen or NH; Z can be a bond (in which case the amide nitrogen is directly bound to the adamantyl group), or the optional linker group illustrated in Formula III, which can be an aliphatic group; and the adamantyl can be substituted or unsubstituted and can have any appropriate stereochemistry. In some embodiments where Z is an aliphatic group, the aliphatic group can comprise one or more methylene groups (—CH— groups), one or more cyclic groups, or a combination thereof (e.g., —C(cyclopentyl)-, —CH₂—C(H)(CH₂)—), wherein the cyclic group can be bound to the adamantyl group or can form a spirocyclic group with the adamantyl group.

In other embodiments, ring A is a three-, four-, five-, or six-membered heteroaliphatic ring system, including monocyclic or multicyclic ring systems, wherein any multicyclic ring systems can comprise fused, spirocyclic, and/or bridged cyclic ring systems. Such heteroaliphatic ring systems can comprise one or more heteroatoms. In particular embodiments, ring A is a five-membered heteroaliphatic ring system, such as a tetrahydrofuran, a pyrrolidine, or a tetrahydrothiophene ring system. In some embodiments, the compound can have a Formula IIIC, including any pharmaceutically acceptable salt, prodrug, hydrate, solvate, or tautomer thereof,

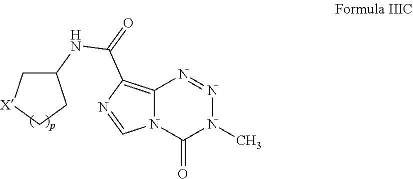

wherein X′ is a selected from O, NH, or S and p is an integer as recited above for Formula IIIA.

In particular embodiments, ring A is a five-, six-, seven-, eight-, nine-, or ten-membered aromatic ring system. In such embodiments, the aromatic ring system can comprise one or more fused rings that are not, themselves, aromatic. For example, the aromatic ring system can be a 2,3-dihydro-1H-indene group. In some embodiments, ring A is a five-, six-, seven-, eight-, nine-, or ten-membered heteroaryl ring system comprising one or more heteroatoms. In some such embodiments, the heteroaryl ring system can be a furan ring system, a thiophene ring system, a pyrrole ring system (e.g., 1H-pyrrole), an oxazole ring system, an isoxazole ring system, a thiazole ring system, an isothiazole ring system, an oxadiazole ring system (e.g., 1,2,5-oxadiazole, 1,2,4-oxadiazole, 1,3,4-oxadiazole, or 1,2,3-oxadiazole), a pyrazole ring system, an imidazole ring system, a triazole ring system (e.g., 1,2,3-triazole), a tetrazole ring system, a thiadiazole ring system (e.g., 1,3,4-thiadiazole or 1,2,5-thiadiazole), and the like. In some embodiments, ring A is a six-, seven-, eight-, nine-, or ten-membered aryl ring system, such as a pyridine ring system, a pyrimidine ring system, a pyridazine ring system, a pyrazine ring system, a triazine ring system, or the like. In some embodiments, the aryl ring system is a phenyl ring that can be unsubstituted or substituted with one or more Y groups, wherein Y can be selected from halogen, haloalkyl, ester, or sulfonamide and wherein such groups can be at the meta, ortho, or para positions on the aryl ring. In embodiments where more than Y group is present, the Y groups can be positioned such that they are bound to adjacent carbon atoms of the phenyl ring, or wherein one or more unsubstituted phenyl carbon atoms are present between the two or more Y groups. For example, in embodiments comprising two Y groups, the Y groups can be bound in a 2,3 or 3,5 relationship. In some embodiments, the compound can have a structure according to Formulas IIIC′ or IIIC″, including any pharmaceutically acceptable salt, prodrug, hydrate, solvate, or tautomer thereof.

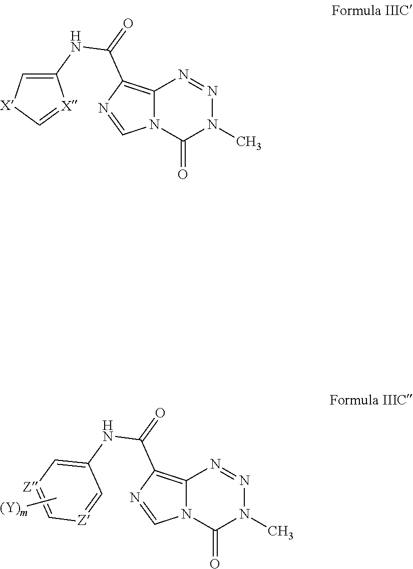

With reference to Formula IIIC′, X′ is selected from O, NH, S; and X″ is selected from O, N, S, or CH. With reference to Formula IIIC″, each of Z′ and Z″ independently is selected from CH, N, or an oxidized N atom.

In some embodiments, the compound can have a structure according to Formulas IIID′ or IIID″, including any pharmaceutically acceptable salt, prodrug, hydrate, solvate, or tautomer thereof.

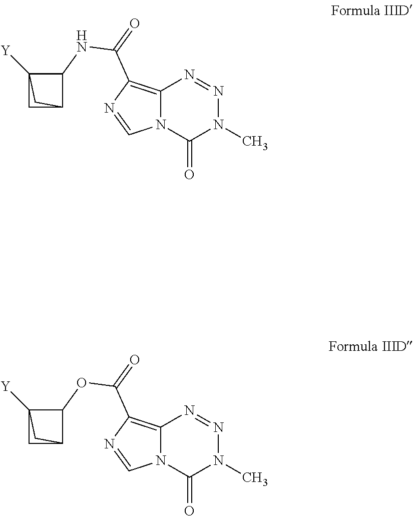

wherein Y is as recited above for any of Formulas III, IIIA′, or IIIA″ and the compound can be either enantiomer.

Non-limiting representative compound embodiments of the present disclosure are provided below.

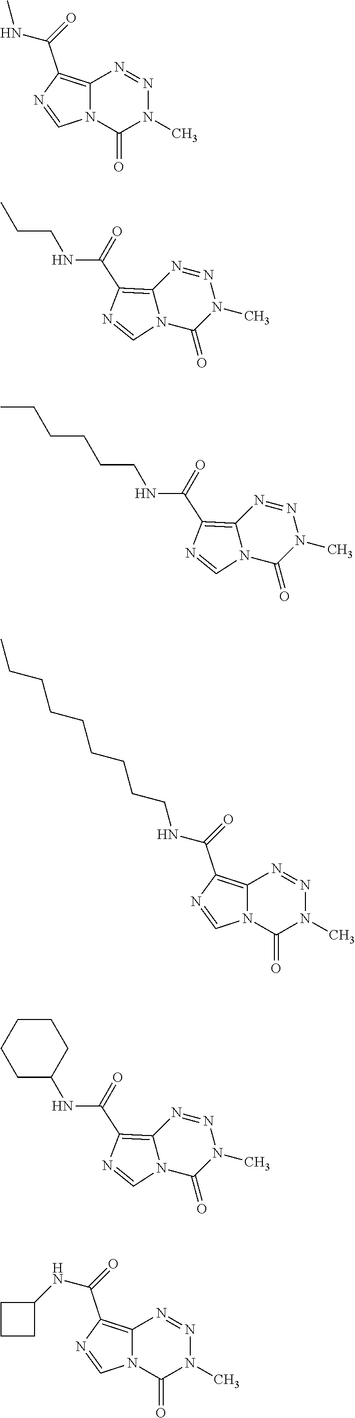

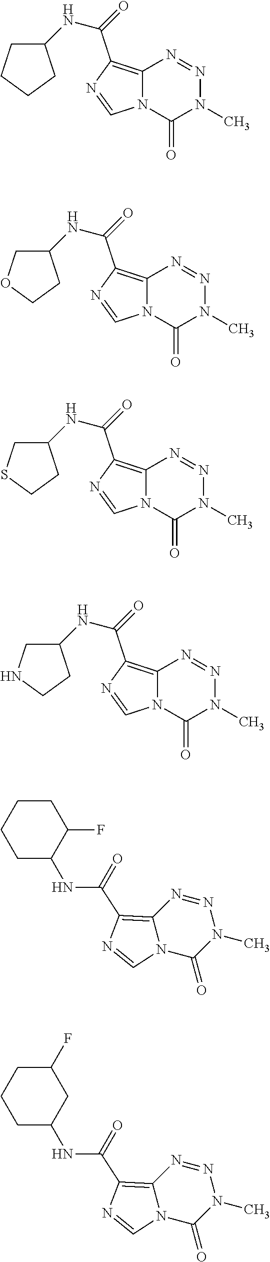 [SMILES: Cn1nnc2c(C(=O)NC3CCCC3)ncn2c1=O ; Cn1nnc2c(C(=O)NC3CCOC3)ncn2c1=O ; Cn1nnc2c(C(=O)NC3CCSC3)ncn2c1=O ; Cn1nnc2c(C(=O)NC3CCNC3)ncn2c1=O ; Cn1nnc2c(C(=O)NC3CCCCC3F)ncn2c1=O ; Cn1nnc2c(C(=O)NC3CCCC(F)C3)ncn2c1=O]

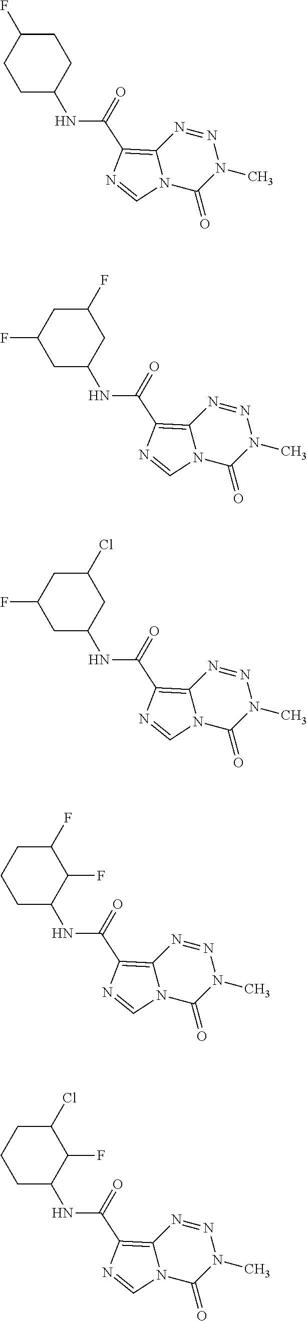 [SMILES: Cn1nnc2c(C(=O)NC3CCC(F)CC3)ncn2c1=O ; Cn1nnc2c(C(=O)NC3CC(F)CC(F)C3)ncn2c1=O ; Cn1nnc2c(C(=O)NC3CC(F)CC(Cl)C3)ncn2c1=O ; Cn1nnc2c(C(=O)NC3CCCC(F)C3F)ncn2c1=O ; Cn1nnc2c(C(=O)NC3CCCC(Cl)C3F)ncn2c1=O]

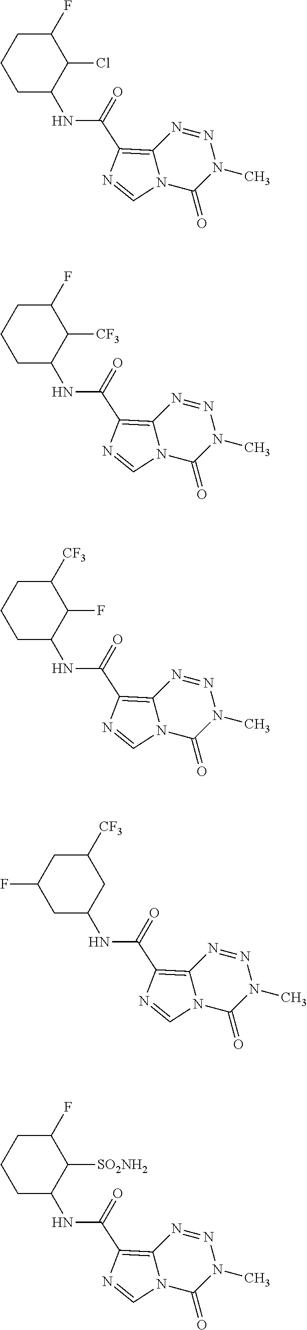 [SMILES: Cn1nnc2c(C(=O)NC3CCCC(F)C3Cl)ncn2c1=O ; Cn1nnc2c(C(=O)NC3CCCC(F)C3C(F)(F)F)ncn2c1=O ; Cn1nnc2c(C(=O)NC3CCCC(C(F)(F)F)C3F)ncn2c1=O ; Cn1nnc2c(C(=O)NC3CC(F)CC(C(F)(F)F)C3)ncn2c1=O ; Cn1nnc2c(C(=O)NC3CCCC(F)C3S(N)(=O)=O)ncn2c1=O]

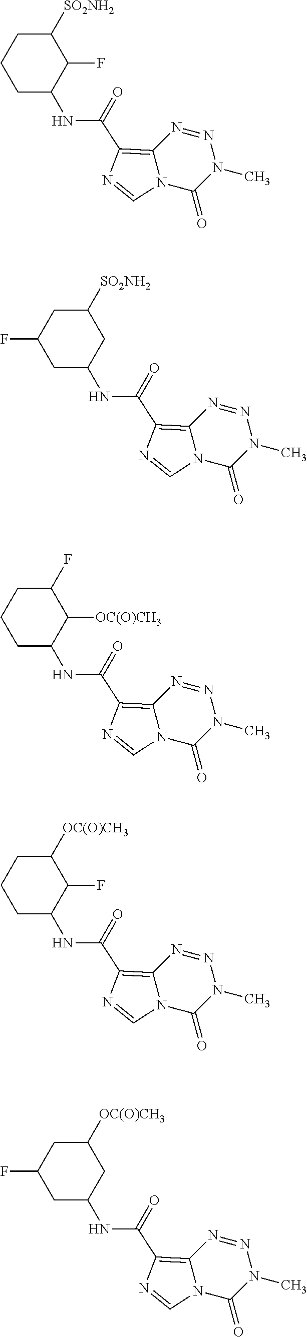

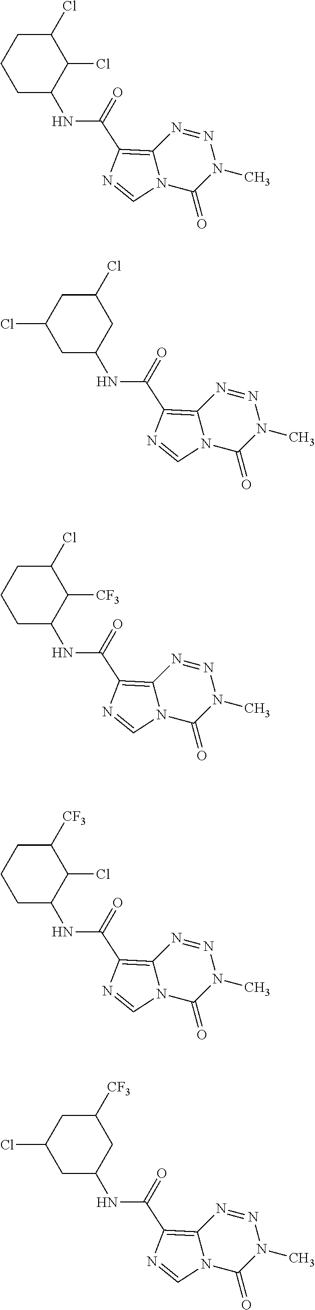 [SMILES: Cn1nnc2c(C(=O)NC3CCCC(Cl)C3Cl)ncn2c1=O ; Cn1nnc2c(C(=O)NC3CC(Cl)CC(Cl)C3)ncn2c1=O ; Cn1nnc2c(C(=O)NC3CCCC(Cl)C3C(F)(F)F)ncn2c1=O ; Cn1nnc2c(C(=O)NC3CCCC(C(F)(F)F)C3Cl)ncn2c1=O ; Cn1nnc2c(C(=O)NC3CC(Cl)CC(C(F)(F)F)C3)ncn2c1=O]

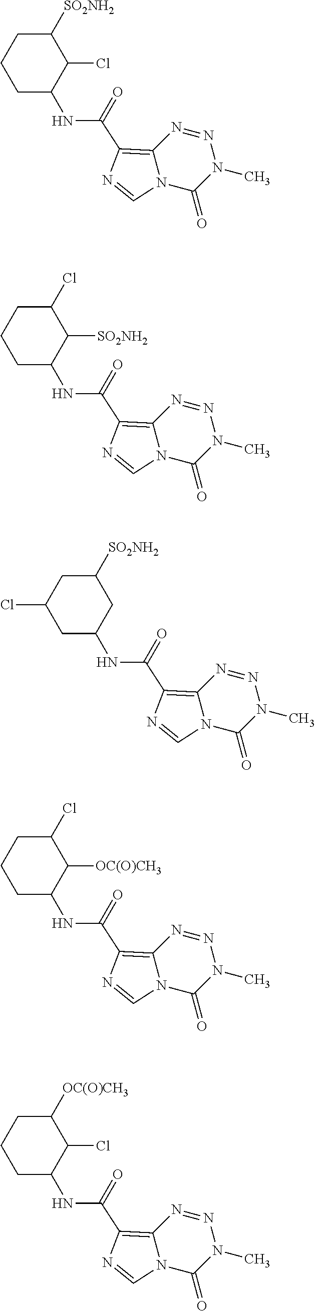

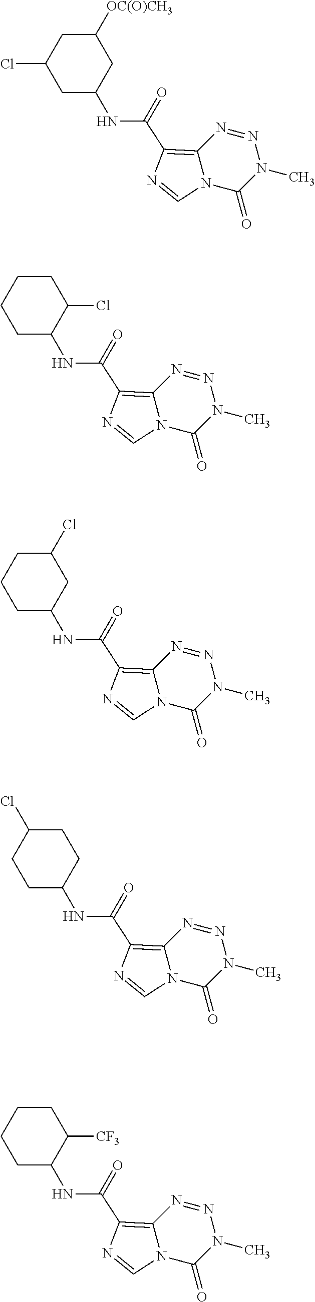

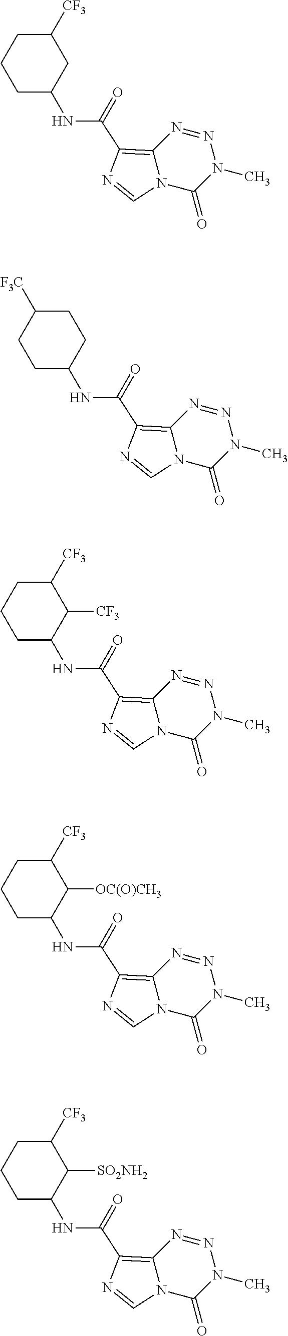

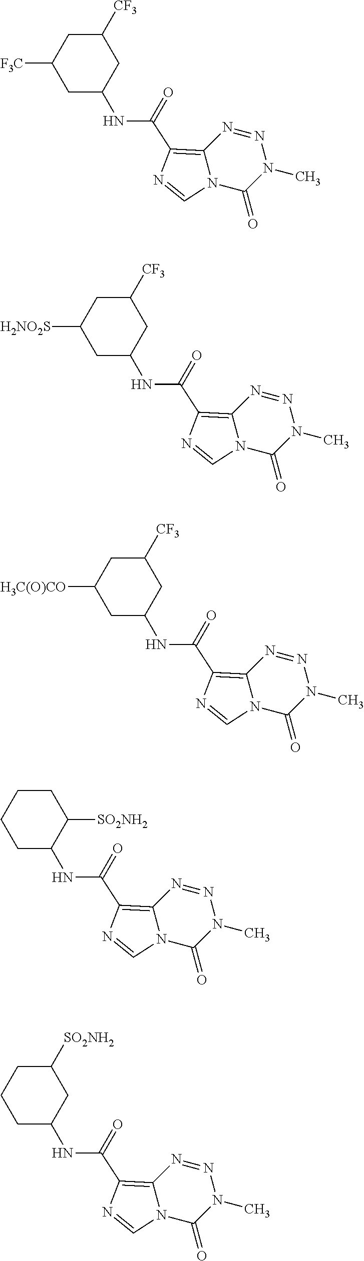

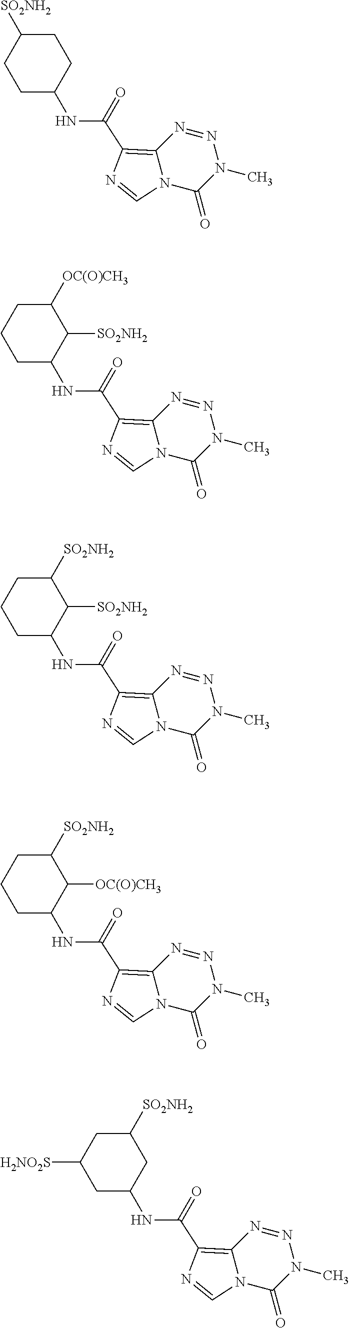

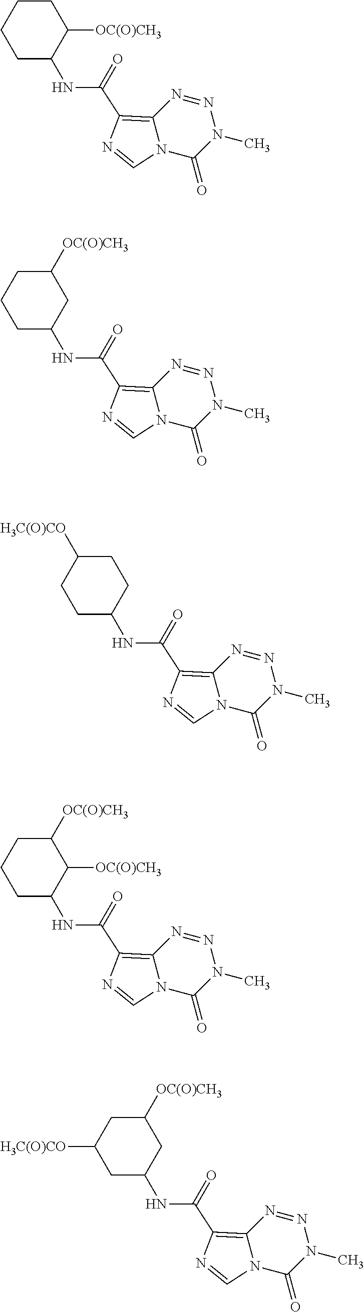

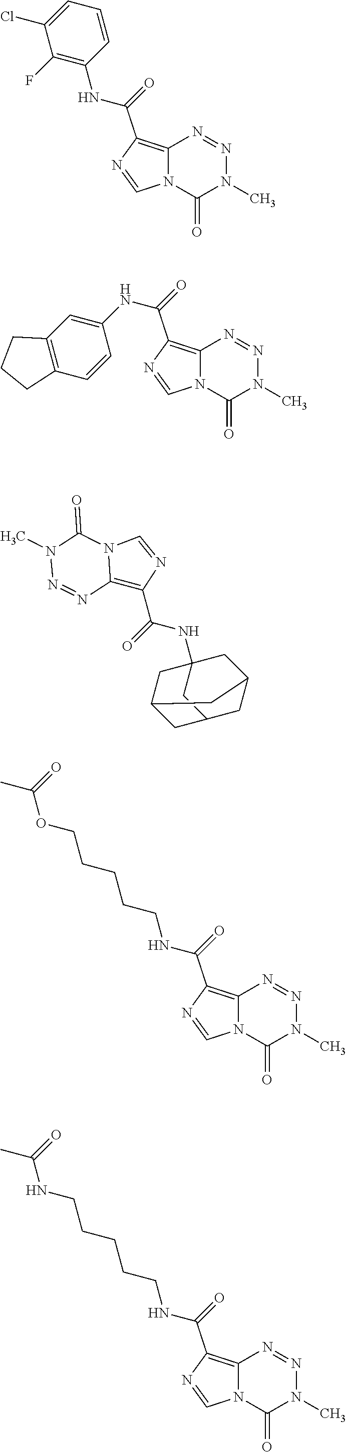

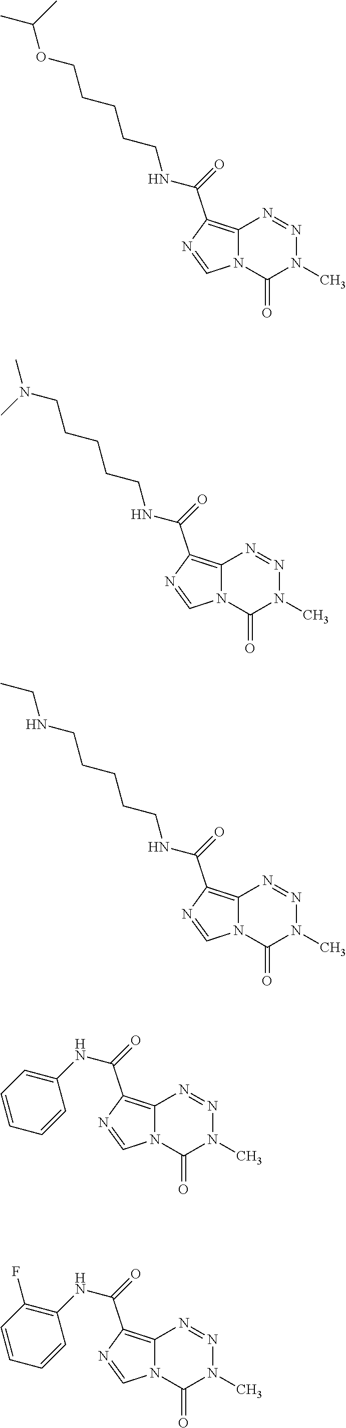

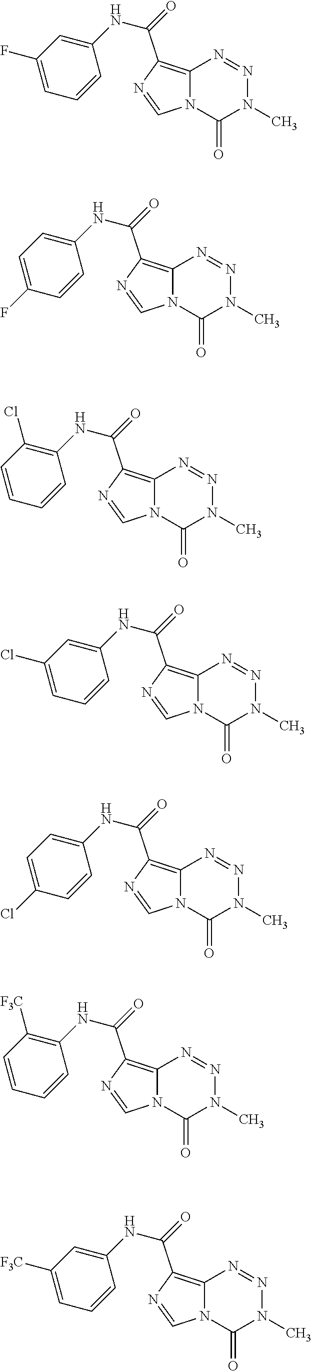

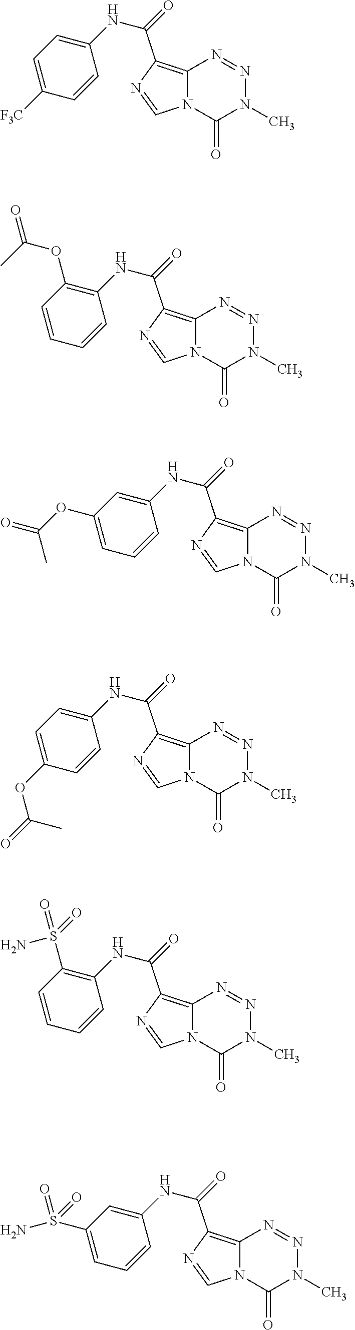

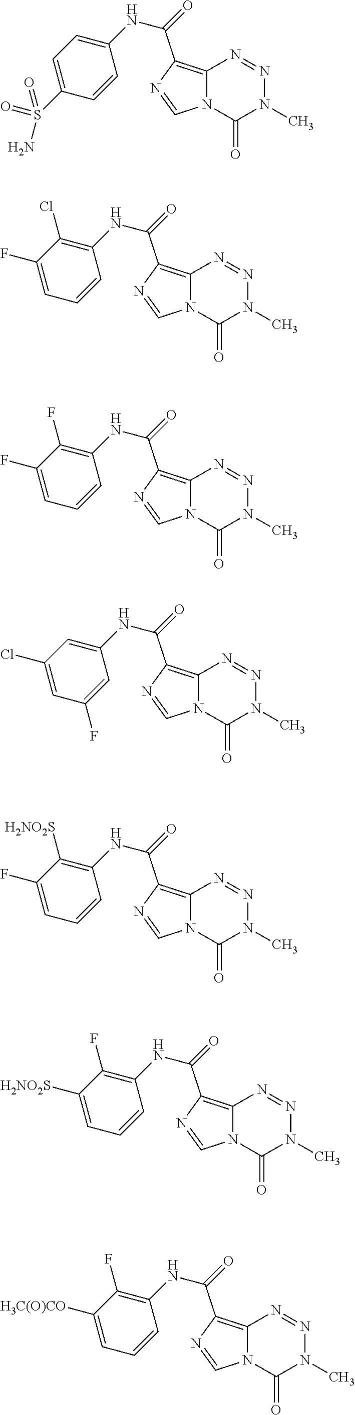

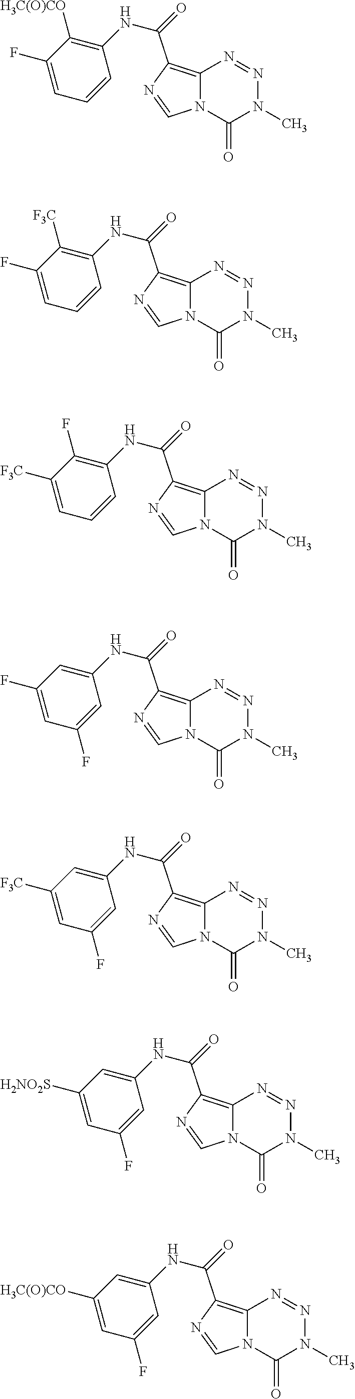

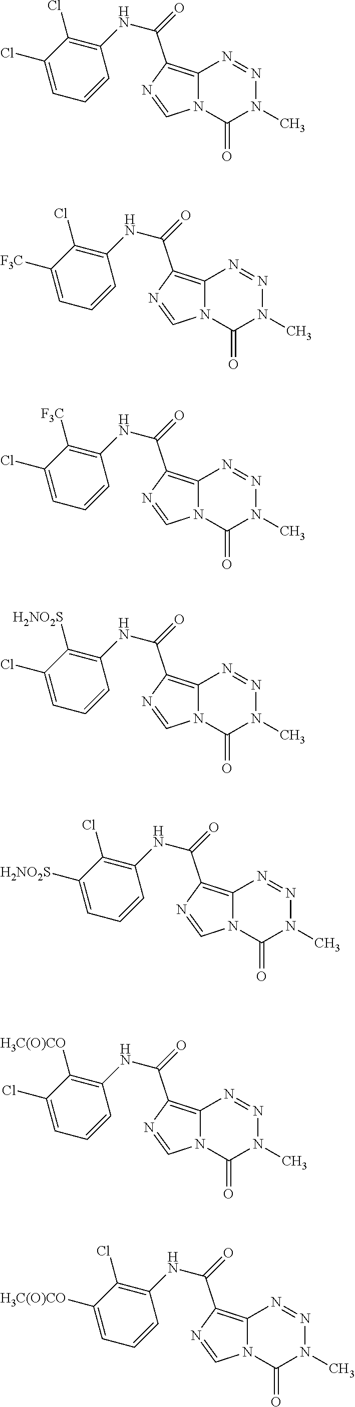

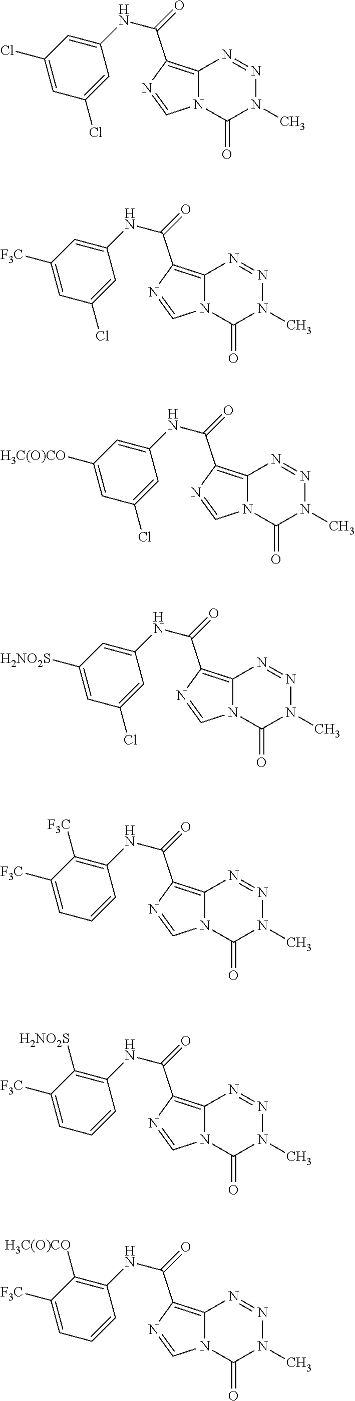

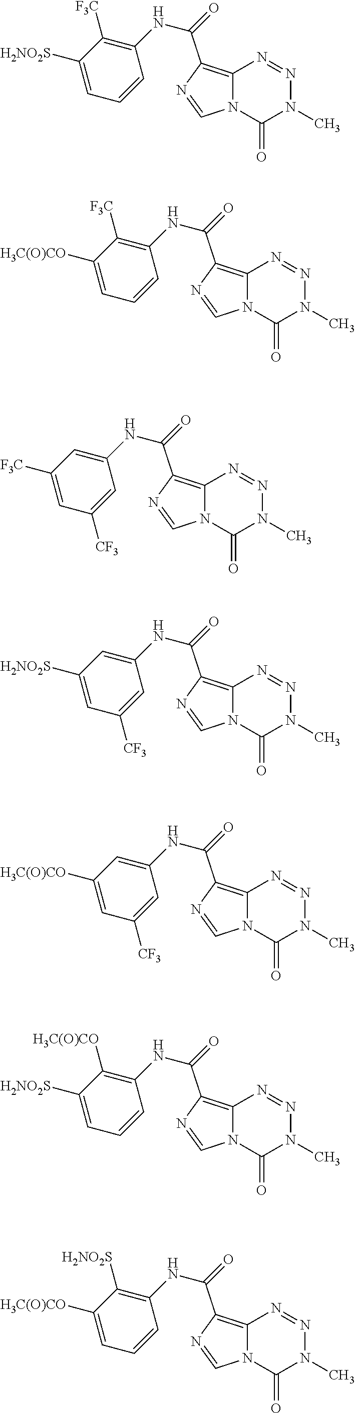

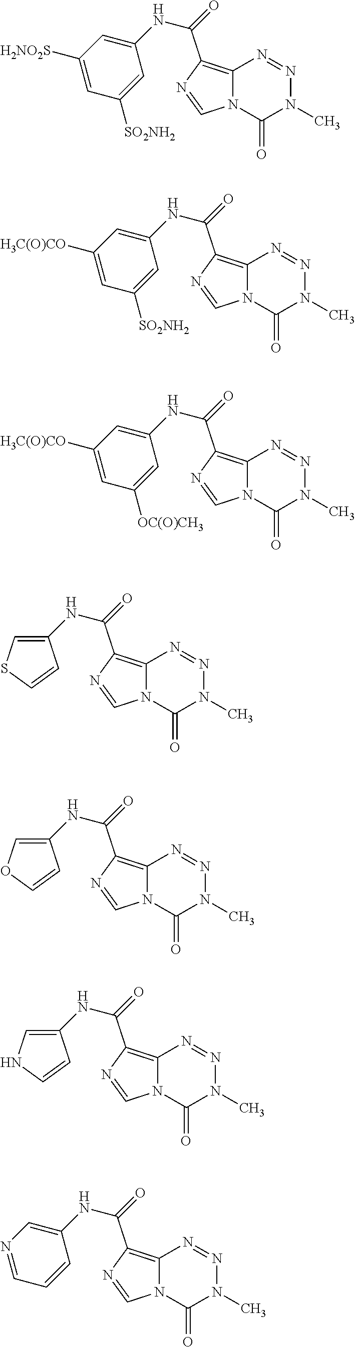

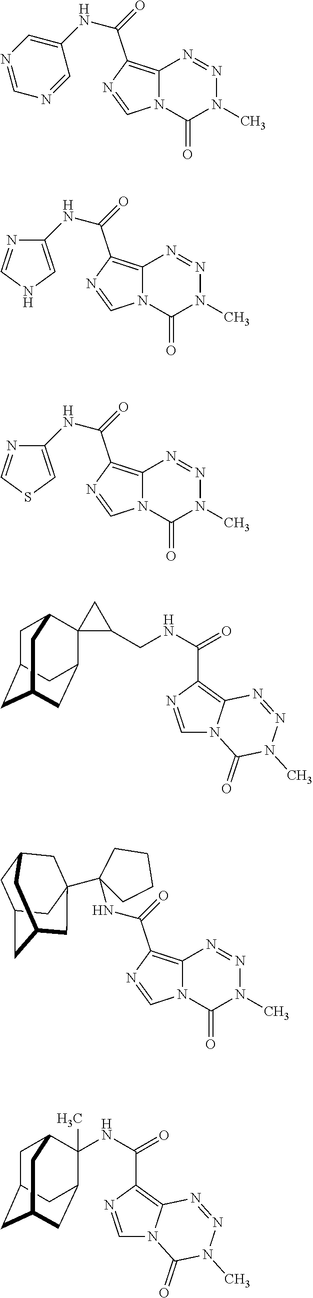

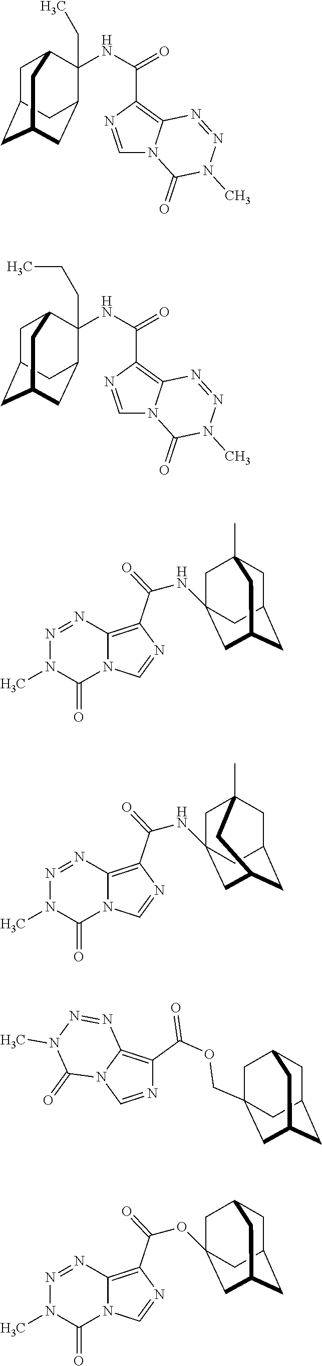

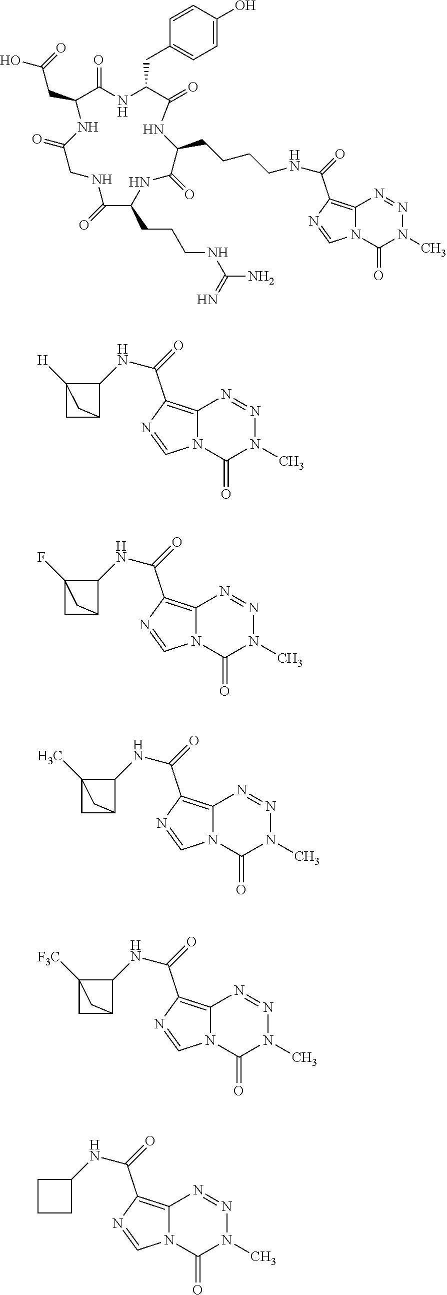

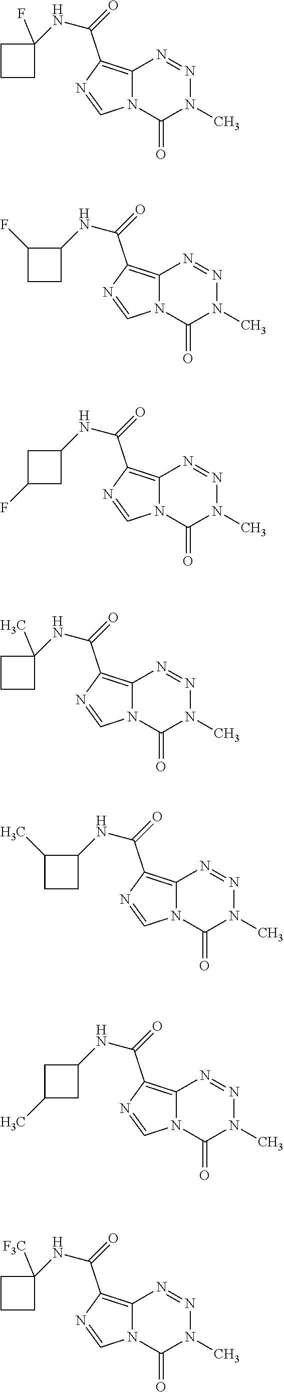

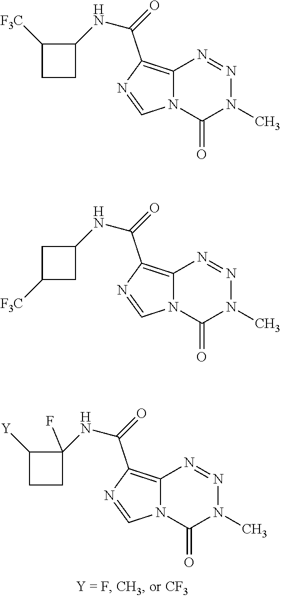

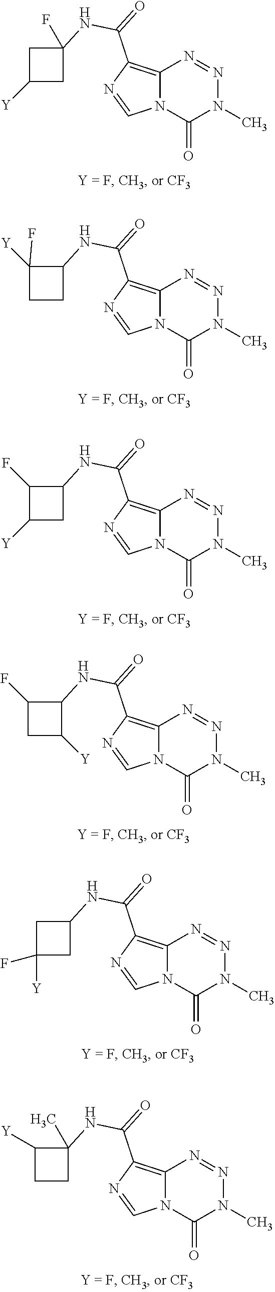

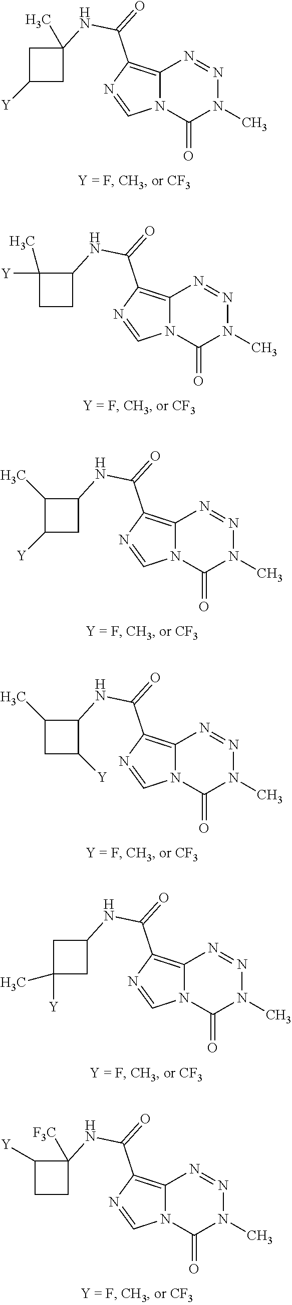

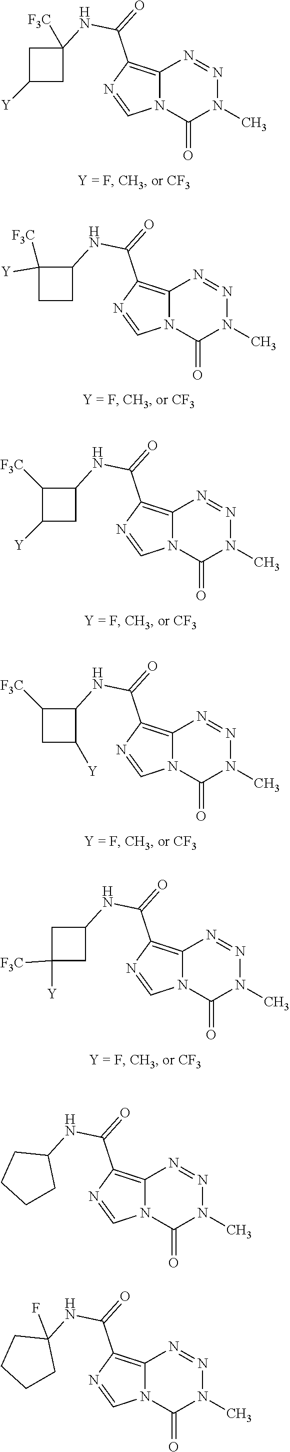

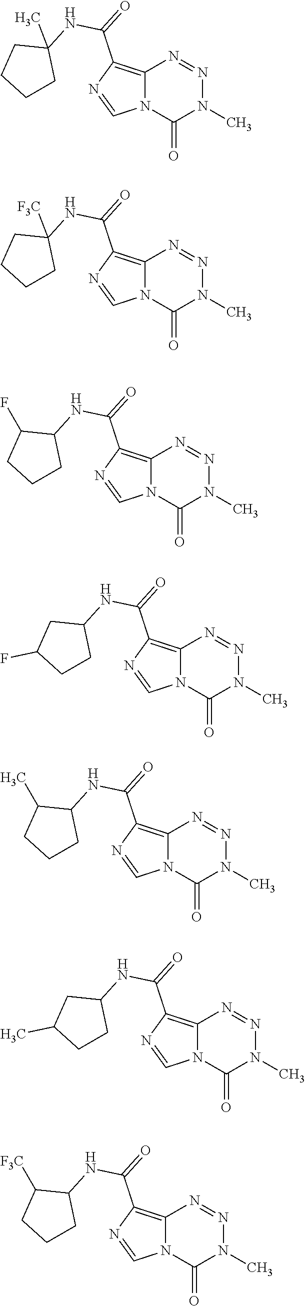

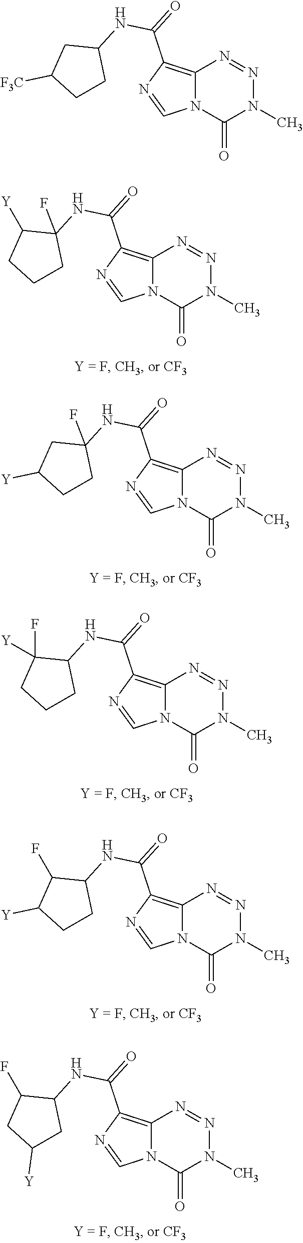

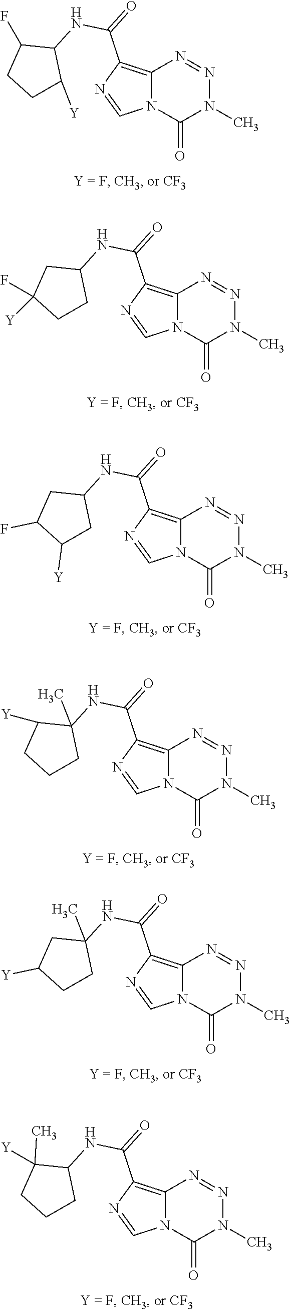

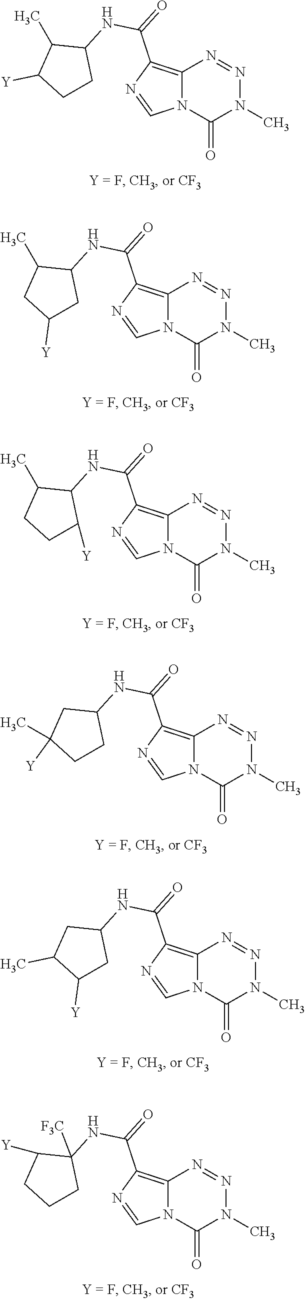

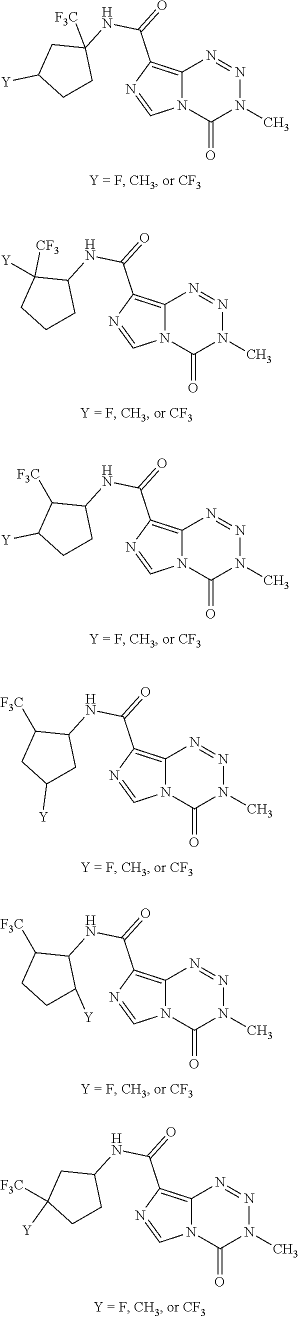

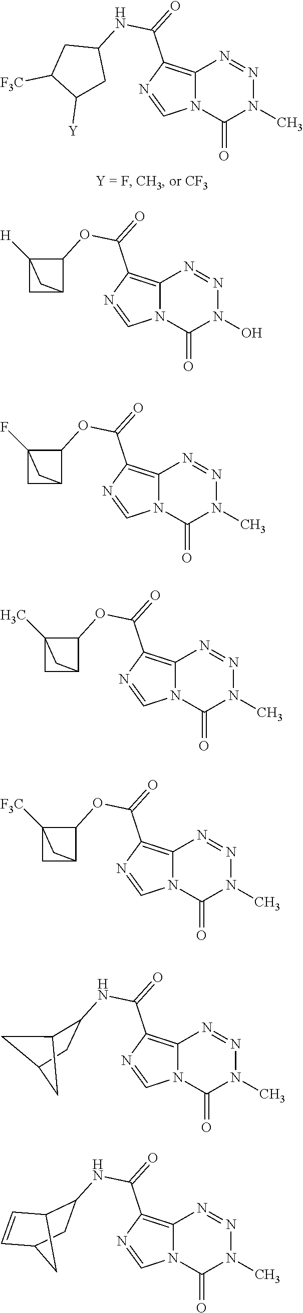

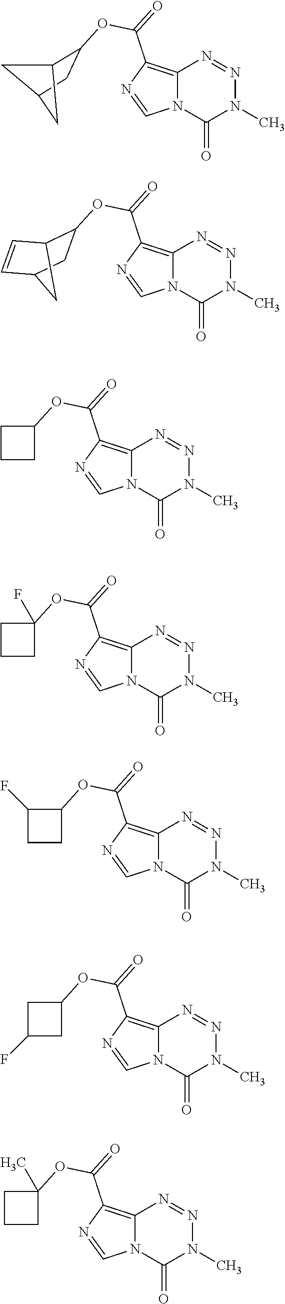

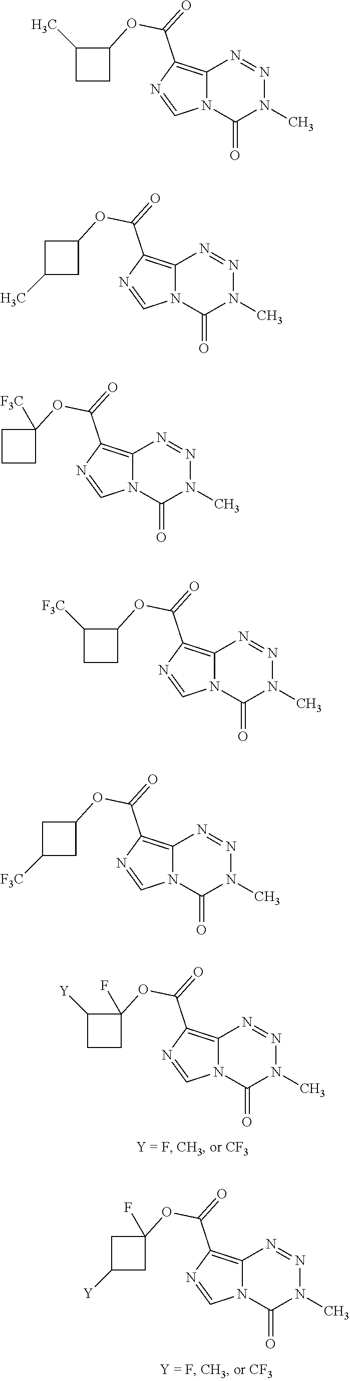

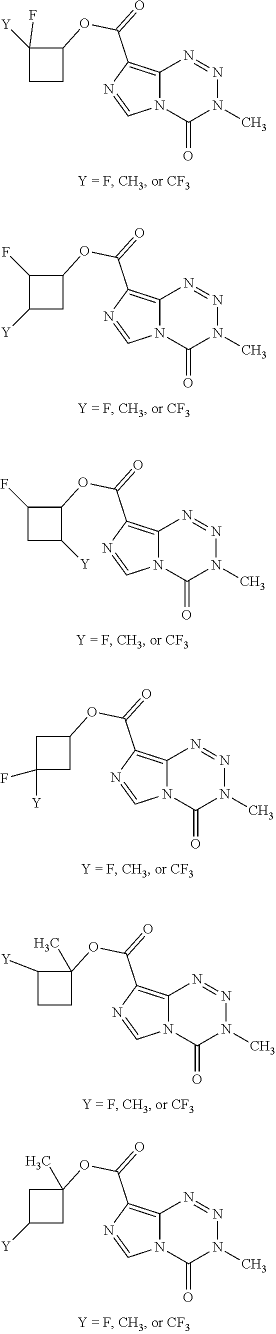

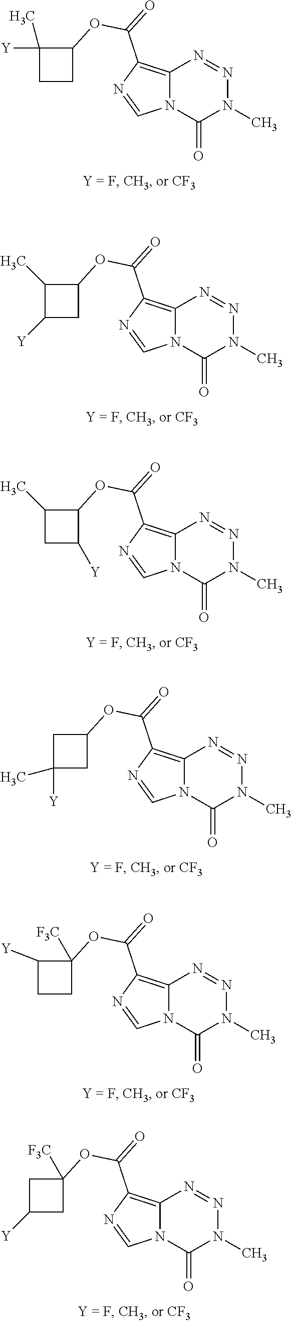

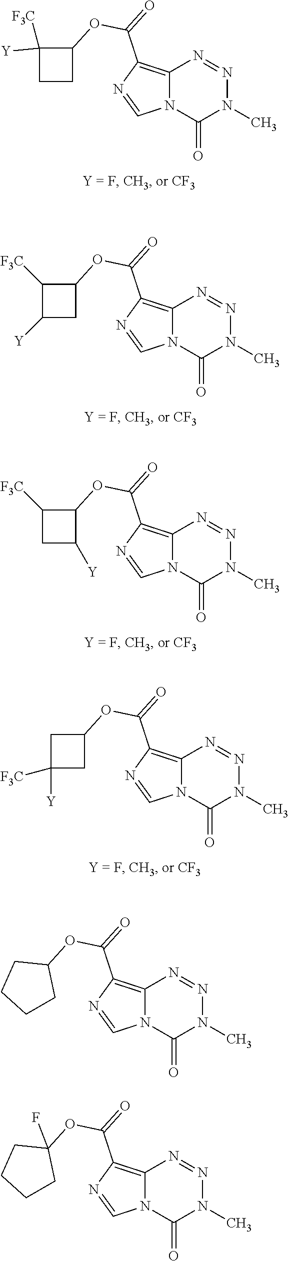

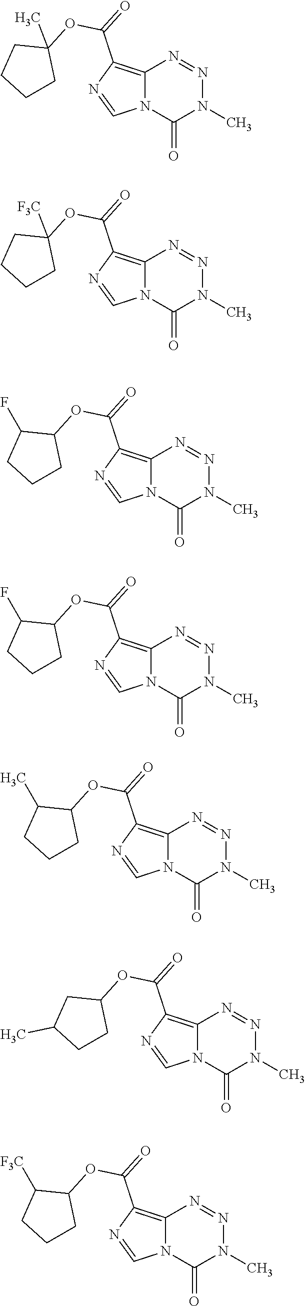

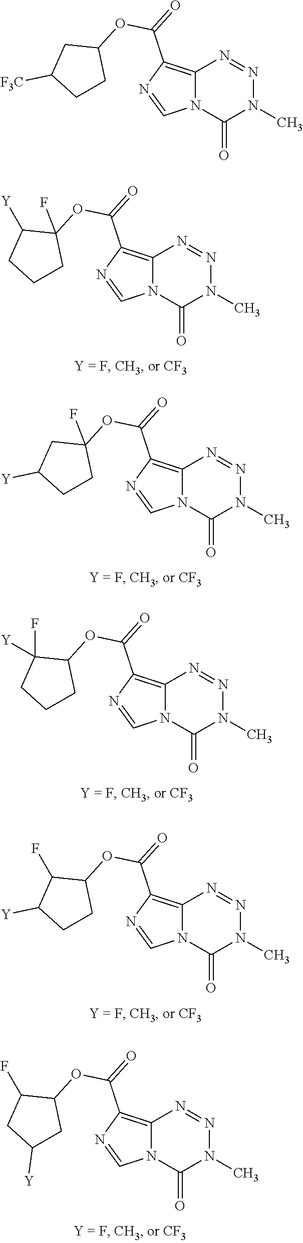

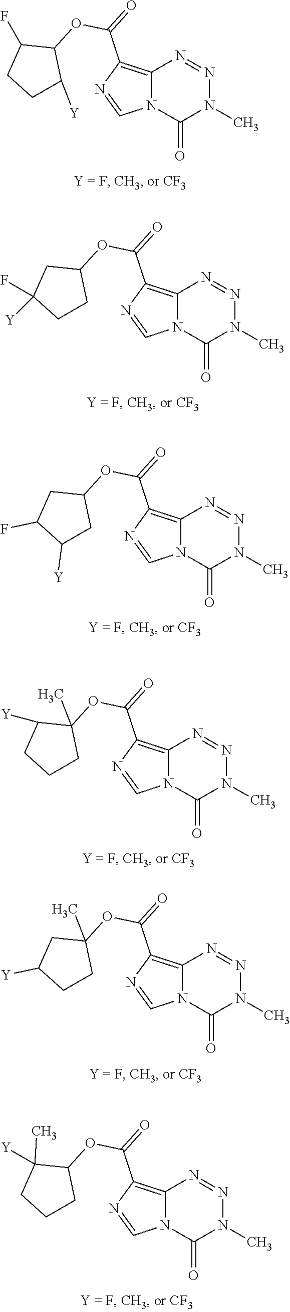

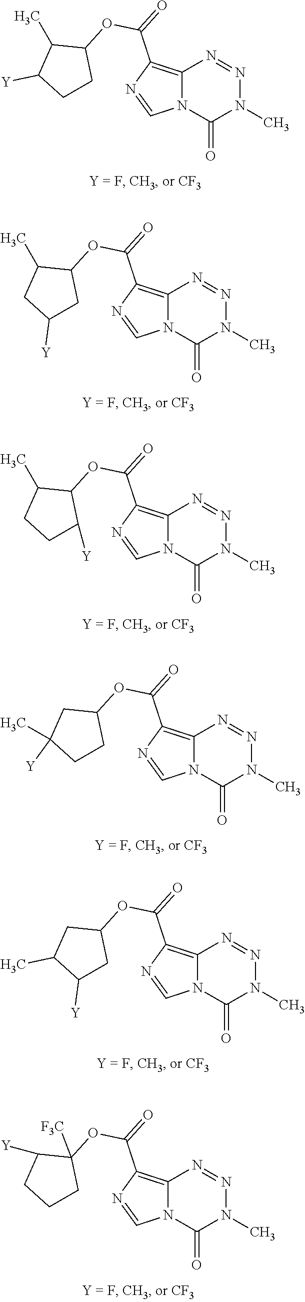

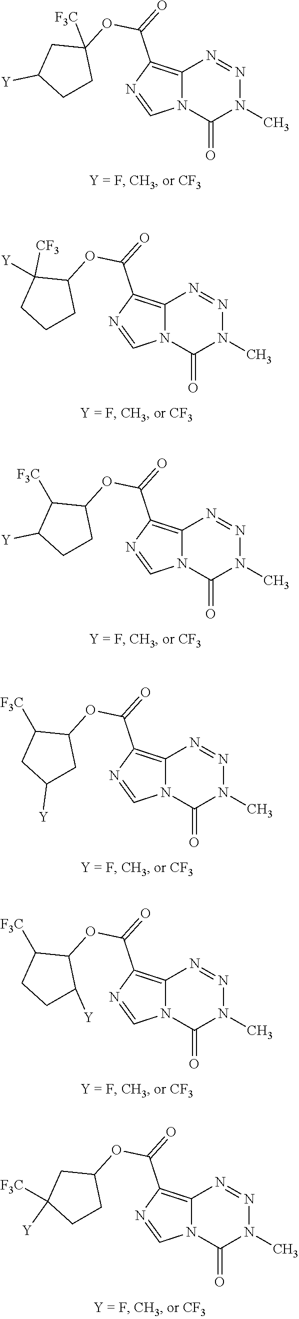

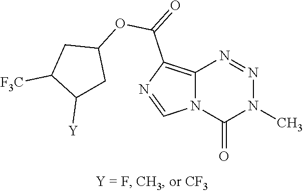

In an independent embodiment, the is not any of the following:

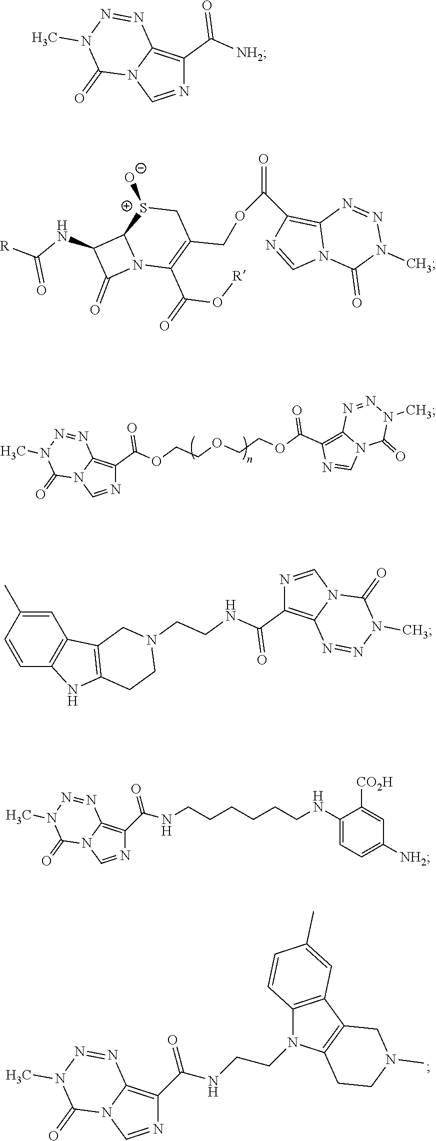

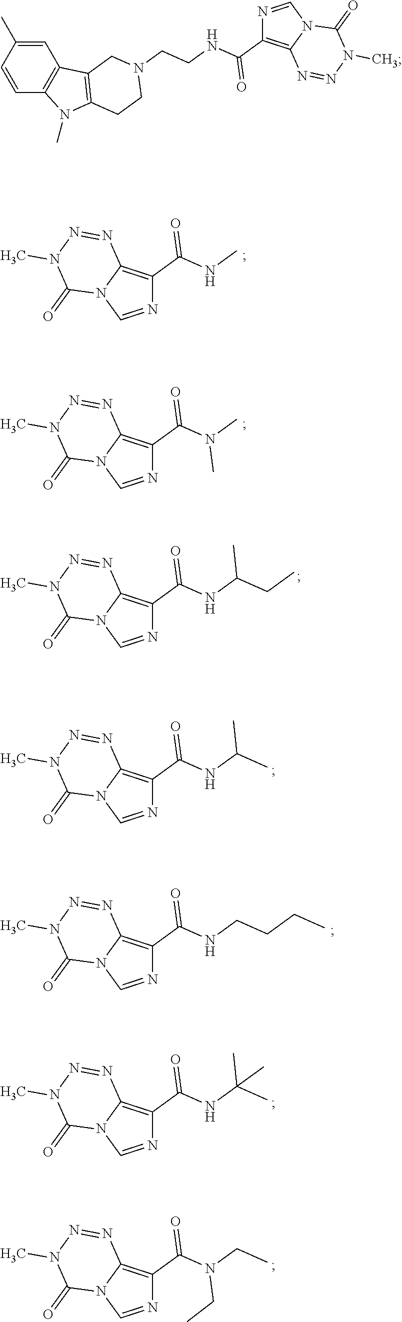

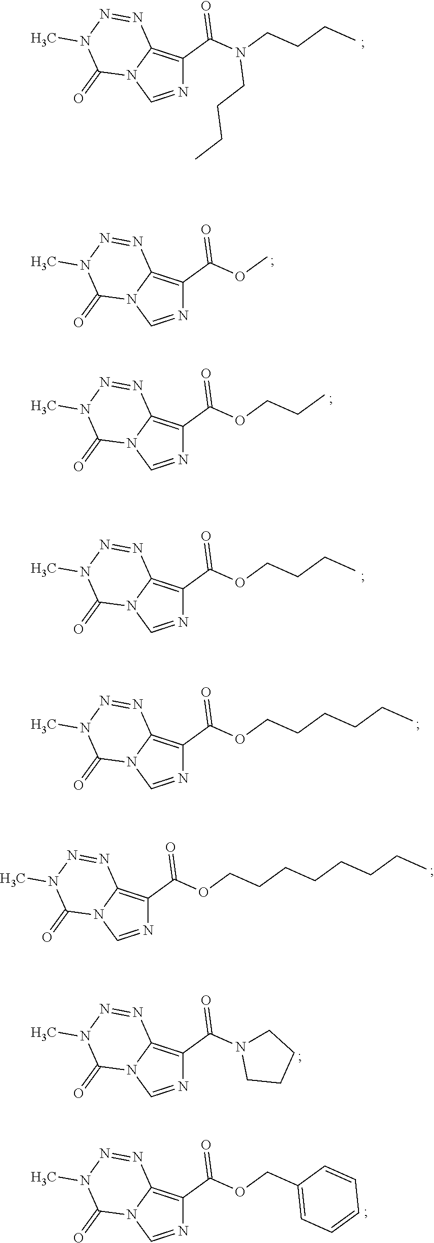

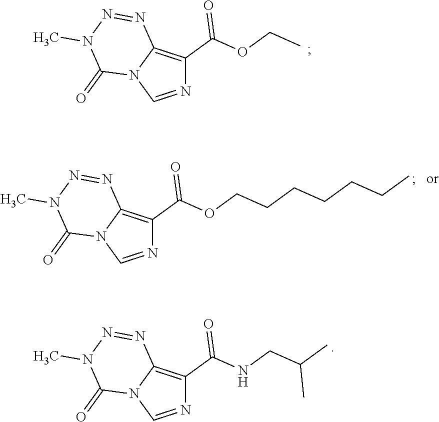

Compound embodiments described herein can be made using methods suitable for forming an amide and/or ester bond between TMZ and an amine- or hydroxyl-terminated compound. For example, in some embodiments, reagents such as diisopropylethylamine, hydroxybenzotriazole, PCl₃, 1-Ethyl-3-(3-dimethylaminopropyl)carbodiimide, and combinations thereof can be used in combination with TMZ and the appropriate coupling partner to provide the desired compound. Exemplary method embodiments are disclosed herein in the Examples section.

### III. Pharmaceutical Compositions and Methods of Treatment

Disclosed herein are methods of treating a subject with one or more compounds provided herein. In some embodiments, the subject has cancer. In some examples, the subject has a cancer that expresses MGMT and/or exhibits MGMT promoter hypomethylation or unmethylation. In one particular non-limiting example, the subject has glioblastoma (e.g., glioblastoma multiforme).

This disclosure includes pharmaceutical compositions including at least one of the compounds described herein for use in human or veterinary medicine. Embodiments of pharmaceutical compositions include a pharmaceutically acceptable carrier and/or excipient and at least one of the disclosed compounds. Useful pharmaceutically acceptable carriers and excipients are known in the art.

The pharmaceutical compositions including one or more of the compounds disclosed herein may be formulated in a variety of ways depending, for example, on the mode of administration and/or the subject or disorder to be treated. For example, pharmaceutical compositions may be formulated as pharmaceutically acceptable salts. As another example, parenteral formulations may comprise injectable fluids that are pharmaceutically and physiologically acceptable fluid vehicles such as water, physiological saline, other balanced salt solutions, aqueous dextrose, glycerol or the like. Excipients may include, for example, nonionic solubilizers, such as Cremophor®, or proteins, such as human serum albumin or plasma preparations. In some examples, the pharmaceutical composition to be administered may also contain non-toxic auxiliary substances, such as wetting or emulsifying agents, preservatives, and pH buffering agents and the like, for example, sodium acetate or sorbitan monolaurate.

Routes of administration include but are not limited to oral and parenteral routes, such as intravenous, intraperitoneal, rectal, topical, ophthalmic, intranasal, and transdermal. The compound may also be delivered intramuscularly or subcutaneously. In particular examples, the compound is administered orally. In other specific examples, the compound is administered intravenously. To extend the time during which the compound is available to inhibit or treat a condition, the compound can be provided as an implant, an oily injection, a liposome, or as a particulate system. The particulate system can be a microparticle, a microcapsule, a microsphere, a nanoparticle, a nanocapsule, or similar particle.

The dosage form of the pharmaceutical composition can be determined, at least in part, by the mode of administration chosen. For example, in addition to injectable fluids, topical or oral formulations may be employed. Topical preparations may include eye drops, ointments, sprays, and the like. Oral formulations may be liquid (e.g., syrups, solutions or suspensions), or solid (e.g., powders, pills, tablets, or capsules). For solid compositions, non-toxic solid carriers include but are not limited to pharmaceutical grade mannitol, lactose, starch, or magnesium stearate.

Pharmaceutical compositions for oral use can also be formulated, for example, as tablets, troches, lozenges, aqueous or oily suspensions, dispersible powders or granules, emulsion hard or soft capsules, or syrups or elixirs. Such compositions may contain one or more agents selected from the group of sweetening agents, flavoring agents, coloring agents and preserving agents. Tablets contain the active ingredient in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatin or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monostearate or glyceryl distearate may be employed. Pharmaceutical compositions for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium such as peanut oil, liquid paraffin, or olive oil.

In some embodiments, a carrier for preparing an oral formulation of a disclosed compound includes Tween 80, glycerol, and a cyclodextrin (such as sulfobutylether-β-cyclodextrin (SBE-β-CD; Captisol®). In one example, the carrier includes 0.4% (v/v) Tween® 80, 2% (v/v) glycerol, and 97.6% (v/v) of 30% (w/v) SBE-β-CD. In other embodiments, a carrier for preparing an oral formulation of a disclosed compound includes a non-ionic surfactant (e.g., caprylocaproyl polyoxyl-8 glycerides (e.g., Labrasol®)), an oil (e.g., transesterified ethoxylated vegetable oil (e.g., Labrafil®)), and a solubilizer (such as diethylene glycol monoethyl ether (e.g., Transcutol®)). In a specific example, the carrier includes 40% (v/v) Labrasol®, 40% (v/v) Labrafil®, and 20% (v/v) Transcutol®. Other carriers that can be used in formulations of the disclosed compounds include polyethylene glycol (e.g., PEG 400), propylene glycol, water (e.g., sterile water), and N,N-dimethylacetamide (DMA). Exemplary formulations are shown in Table 12. An additional exemplary carrier includes 10% DMSO in PBS.

The disclosed compounds can be conveniently presented in unit dosage form and prepared using techniques known to one of skill in the art. Such techniques include the step of bringing into association the active ingredient and the pharmaceutical carrier(s) or excipient(s). The formulations may be included in unit-dose or multi-dose containers, for example, sealed ampoules and vials, and may be stored in a dried condition requiring only the addition of a sterile liquid carrier, for example, water or saline for injections, immediately prior to use. In certain embodiments, unit dosage formulations are those containing a dose or unit, or an appropriate fraction thereof, of the administered ingredient.

The amount of the compound that will be effective depends on the nature of the disorder or condition to be treated, as well as the stage of the disorder or condition. Effective amounts can be determined by in vitro studies, animal studies, and clinical techniques. The precise dose of the compounds to be included in the formulation will also depend on the route of administration, and should be decided according to the judgment of the health care practitioner and each subject's circumstances. An example of such a dosage range is 1 μg/kg to 200 mg/kg body weight (for example, about 5 μg/kg to 1 mg/kg, about 10 μg/kg to 5 mg/kg, about 100 μg/kg to 20 mg/kg, about 0.2 to 100 mg/kg, about 0.5 to 50 mg/kg, about 1 to 25 mg/kg, about 5 to 75 mg/kg, about 50 to 150 mg/kg, or about 100 to 200 mg/kg) in single or divided doses. For example, a suitable dose may be about 0.1 mg/kg, about 0.2 mg/kg, about 0.5 mg/kg, about 0.75 mg/kg, about 1 mg/kg, about 2 mg/kg, about 5 mg/kg, about 7.5 mg/kg, about 10 mg/kg, about 15 mg/kg, about 20 mg/kg, about 25 mg/kg, about 30 mg/kg, about 40 mg/kg, about 50 mg/kg, about 75 mg/kg, about 100 mg/kg, about 150 mg/kg, or about 200 mg/kg.

One or more doses of the compound can be administered to a subject. For example, the compound can be administered three times per day, twice per day, daily, every other day, twice per week, weekly, every other week, every three weeks, monthly, or less frequently. In some examples, the compound may be administered in one or more cycles, for example, at a set interval (such as weekly or daily) for a set number of intervals, followed by a rest period, then repeated one or more times.

The specific dose level and frequency of dosage for any particular subject may be varied and will depend upon a variety of factors, including the disorder being treated, the specific compound being administered, the age, body weight, general health, sex and diet of the subject, mode and time of administration, and so on.

In some embodiments, the subject being treated has a solid tumor. Examples of solid tumors include sarcomas (such as fibrosarcoma, myxosarcoma, liposarcoma, chondrosarcoma, osteosarcoma, soft tissue sarcoma, and other sarcomas), synovioma, mesothelioma, Ewing sarcoma, leiomyosarcoma, rhabdomyosarcoma, colon cancer, colorectal cancer, peritoneal cancer, esophageal cancer (such as esophageal squamous cell carcinoma), pancreatic cancer, breast cancer (including basal breast carcinoma, ductal carcinoma and lobular breast carcinoma), endometrial cancer, lung cancer (such as non-small cell lung cancer), ovarian cancer, prostate cancer, liver cancer (including hepatocellular carcinoma), gastric cancer, squamous cell carcinoma (including head and neck squamous cell carcinoma), basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, hepatoma, bile duct carcinoma, choriocarcinoma, Wilms tumor, cervical cancer, fallopian tube cancer, testicular tumor, seminoma, bladder cancer (such as renal cell cancer), melanoma, and CNS tumors (such as a glioma, glioblastoma, astrocytoma, medulloblastoma, craniopharyrgioma, ependymoma, pinealoma, hemangioblastoma, acoustic neuroma, oligodendroglioma, meningioma, neuroblastoma and retinoblastoma). Solid tumors also include tumor metastases (for example, metastases to the lung, liver, brain, or bone).

In other examples, the subject has a hematological malignancy. Examples of hematological malignancies include leukemias, including acute leukemias (such as 11q23-positive acute leukemia, acute lymphocytic leukemia (ALL), T-cell ALL, acute myelocytic leukemia, acute myelogenous leukemia (AML), and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), lymphoblastic leukemia, polycythemia vera, lymphoma, diffuse large B cell lymphoma, Burkitt lymphoma, T cell lymphoma, follicular lymphoma, mantle cell lymphoma, Hodgkin disease, non-Hodgkin lymphoma, multiple myeloma, Waldenstrom macroglobulinemia, heavy chain disease, myelodysplastic syndrome, hairy cell leukemia, and myelodysplasia.

In particular examples, the subject has a glioma, such as glioblastoma multiforme, anaplastic glioma, anaplastic oligodendroglioma, astrocytoma, oligodendroglioma, or pleomorphic xanthoastrocytoma. In other examples, the subject has breast cancer, gastric cancer, colorectal cancer, head and neck cancer, melanoma, lung cancer, pancreatic cancer, bladder cancer, or prostate cancer. In other examples, the subject has acute myeloblastic leukemia. In additional examples, the subject has a solid tumor or hematological malignancy that expresses or overexpresses MGMT and/or has hypomethylation or unmethylation of MGMT promoter.

In some examples, the subject with cancer is also treated with surgery, radiation therapy, chemotherapeutic agents, immunotherapy, or any combination thereof. A skilled clinician can select an appropriate combination of additional treatments with the TMZ analogs provided herein, based on the type of cancer being treated. In one non-limiting example, a subject with GBM is treated with compound disclosed herein and radiation therapy.

### EXAMPLES

The following examples are provided to illustrate certain features and/or embodiments. These examples should not be construed to limit the disclosure to the particular features or embodiments described.

### Example 1

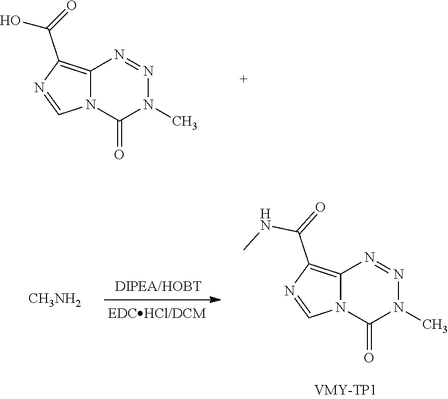

N,3-dimethyl-4-oxo-3,4-dihydroimidazo[5,1-d][1,2,3,5]tetrazine-8-carboxamide (VMY-TP1): To a stirred solution of Temozolomide acid (200 mg, 0.0001 mmol.) in DCM (1 mL, 5 vol), was added DIPEA (0.5 mL, 0.0003 mmol), HOBt (0.46 mL, 0.0024 mmol.), EDC-HCl (294 mg, 0.0001) and cooled to 10-15° C. To the reaction mass, was added methyl amine (80 mg, 0.00002 mmol.) and allowed to stir at 25-30° C. for 15 hours. The reaction completion was monitored by LC-MS. Upon reaction completion, reaction mass was concentrated under reduced pressure to get crude compound. The crude compound was purified by prep-HPLC; pure fractions where collected and dried under high vacuum to get VMY-TP1 as a white solid (40 mg, 18%). HRMS (ESI): exact mass calculated for C₇H₈N₆O₂[M+H]⁺, 209.07, found 209.1. ¹H NMR (400 MHz, DMSO-d₆) δ: 8.84 (s, 1H), 8.46 (brd, 1H), 3.86 (s, 3H), 2.81 (d, J=4.8 Hz, 3H).

### Example 2

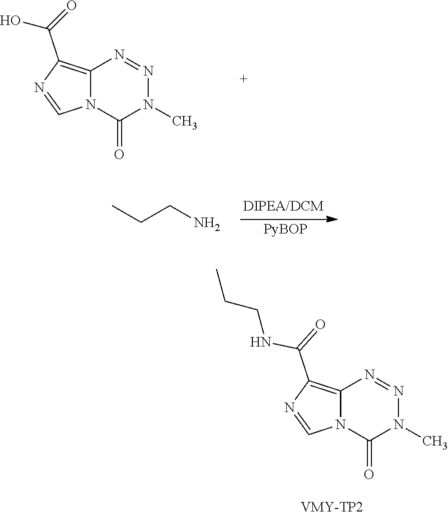

3-methyl-4-oxo-N-propyl-3,4-dihydroimidazo[5,1-d][1,2,3,5]tetrazine-8-carboxamide (VMY-TP2): To a stirred solution of temozolomide acid (175 mg, 0.0009 mmol.) in DCM (1.7 mL, 10 vol), was added propyl amine (80 mg, 0.0013 mmol) DIPEA (0.46 mL, 0.0024 mmol.) and cooled to 0-5° C. To the reaction mass, was added Py-Bop (606 mg, 0.0011 mmol.) and allowed to stir at 25-30° C. for 15 hours. The reaction completion was monitored by LC-MS. Upon reaction completion, reaction mass was concentrated under reduced pressure to get crude compound. The crude compound was purified by prep-HPLC; pure fractions where collected and dried under high vacuum to get VMY-TP1 as pale yellow solid (80 mg, 31.5%). ¹H NMR (400 MHz, DMSO-d₆) δ: 8.84 (s, 1H), 8.49 (m, 1H), 3.86 (s, 3H), 3.25 (d, J=8 Hz, 2H), 1.57-1.51 (m, 2H), 0.87 (t, J=7.2 Hz, 3H). HRMS (ESI): exact mass calculated for C₉H₁₂N₆O₂ [M+H]⁺, 237.10, found 237.1.

### Example 3

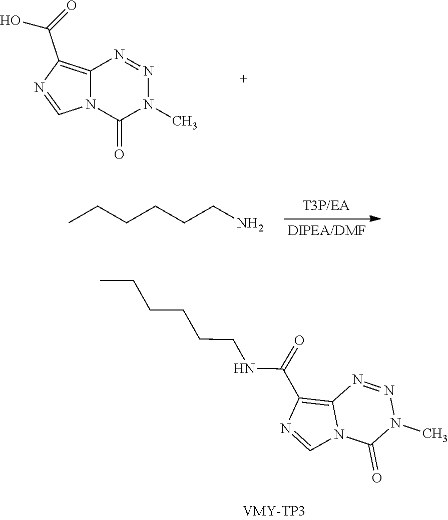

N-hexyl-3-methyl-4-oxo-3,4-dihydroimidazo[5,1-d][1,2,3,5]tetrazine-8-carboxamide (VMY-TP3): To a stirred solution of temozolomide acid (0.3 g, 0.0015 mol.) in ethyl acetate (6.0 mL, 20.0 vol.) and DMF (1.5 mL, 5.0 vol.), was added DIPEA (1.33 mL, 0.0076 mol.) followed by hexyl amine (0.18 g, 0.0018 mol.). Reaction mass was cooled to 0-5° C., and propylphosphonic anhydride (50% solution in ethyl acetate, 2.9 mL, 0.0046 mol.) was added dropwise by maintaining temperature<10° C. After complete addition, the reaction mixture was stirred at 25-30° C. for 2 hours. The completion of the reaction was monitored by LCMS. Upon reaction completion, the reaction mixture was cooled to 0-5° C. and quenched with water (1.5 mL, 5.0 vol). The layers were separated and the organic layer was dried over Na₂SO₄ and concentrated under reduced pressure to get crude product (0.45 g). The crude product was purified by preparative HPLC to get pure VMY-TP3 as an off-white solid (0.2 g, 46.8%). ¹H NMR (400 MHz, DMSO-d₆) δ: 8.84 (s, 1H), 8.48 (t, J=6 Hz, 1H), 3.86 (s, 1H), 3.28 (q, J=13.6, 6.8 Hz, 2H), 1.54-1.48 (m, 2H), 1.35-1.22 (m, 6H), 0.90-0.82 (m, 3H). HRMS (ESI): exact mass calculated for C₁₂H₁₈N₆O₂ [M+H]⁺, 279.15, found 279.5.

### Example 4

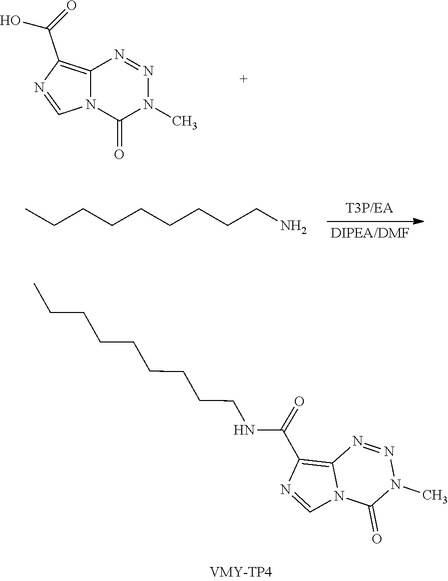

3-methyl-N-nonyl-4-oxo-3,4-dihydroimidazo[5,1-d][1,2,3,5]tetrazine-8-carboxamide (VMY-TP4): To a stirred solution of temozolomide acid (160 mg, 0.0008 mmol.) in EA (1.6 mL, 10 vol), was added DMF (0.8 mL, 5.0 vol) followed by DIPEA (0.4 mL, 0.0025 mmol.) and cooled to 0-5° C. To the reaction mass, was added propylphosphonic anhydride (2.6 mL, 0.0041 mmol.) and allowed to stir at 25-30° C. for 4 hours. The reaction completion was monitored by LC-MS. Upon reaction completion, the reaction mixture was diluted with EA (1.6 mL, 10 vol) and quenched with water (1.6 mL, 10 vol). The layers were separated and the organic layer was washed with sat. sodium chloride solution and separated layers. The organic layer was dried over anhydrous sodium sulphate and concentrated under reduced pressure to get VMY-TP4 as white solid (61 mg, 23.1%). HRMS (ESI): exact mass calculated for C₁₅H₂₄N₆O₂ [M+H]⁺, 321.20, found 321.7.

### Example 5

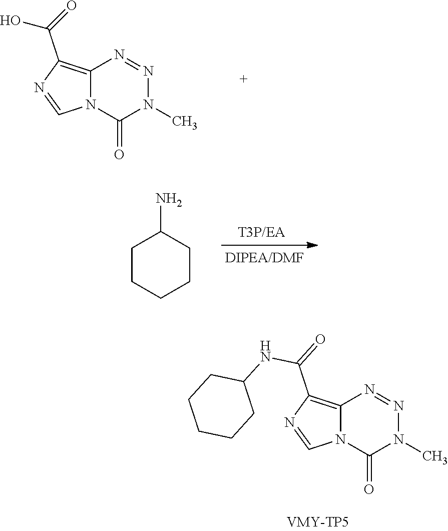 [SMILES: Cn1nnc2c(C(=O)O)ncn2c1=O ; NC1CCCCC1 ; Cn1nnc2c(C(=O)NC3CCCCC3)ncn2c1=O]

N-cyclohexyl-3-methyl-4-oxo-3,4-dihydroimidazo[5,1-d][1,2,3,5]tetrazine-8-carboxamide (VMY-TP5): To a stirred solution of temozolomide acid (100 mg, 0.0005 mmol.) in EA (1.0 mL, 10 vol), was added cyclohexylamine (80 mg, 0.0006 mmol), DMF (0.5 mL, 5.0 vol) followed by DIPEA (0.5 mL, 0.0025 mmol.) and cooled to 0-5° C. To the reaction mass, was added propylphosphonic anhydride (0.9 mL, 0.0025 mmol.) and allowed to stir at 25-30° C. for 5 hours. The reaction completion was monitored by LC-MS. Upon reaction completion, the reaction mixture was diluted with EA (1.6 mL, 10 vol) and quenched with water (1.6 mL, 10 vol). The layers were separated and the organic layer was washed with sat. sodium chloride solution and separated layers. The organic layer was dried over anhydrous sodium sulphate and concentrated under reduced pressure to get VMY-TP5 as white solid (70 mg, 50%). HRMS (ESI): exact mass calculated for C₁₂H₁₆N₆O₂ [M+H]⁺, 277.13, found 277.2. ¹H NMR (400 MHz, DMSO-d₆) δ: 8.84 (s, 1H), 8.15 (d, J=8.4 1H), 3.86 (s, 3H), 3.84-3.79 (m, 1H), 1.81-1.70 (m, 4H), 1.61-1.57 (m, 1H), 1.45-1.24 (m, 4H), 1.20-1.04 (m, 1H).

### Example 6

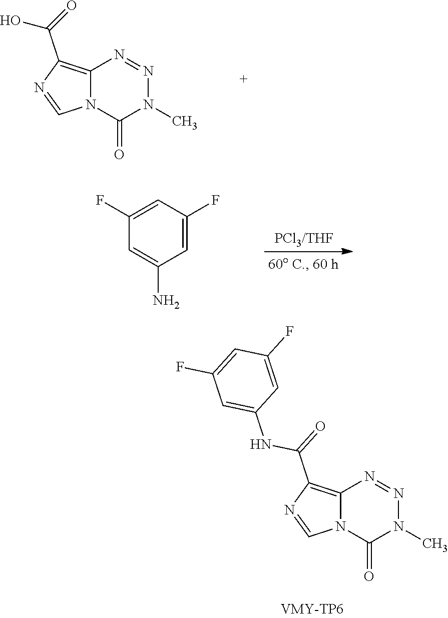 [SMILES: Cn1nnc2c(C(=O)O)ncn2c1=O ; Nc1cc(F)cc(F)c1 ; Cn1nnc2c(C(=O)Nc3cc(F)cc(F)c3)ncn2c1=O]

N-(3,5-difluorophenyl)-3-methyl-4-oxo-3,4-dihydroimidazo[5,1-d][1,2,3,5]tetrazine-8-carboxamide (VMY-TP6): To a stirred solution of temozolomide acid (250 mg, 1.2811 mmol.) and 3,5-difluoroaniline (198 mg, 1.5373 mmol.) in THF (5 mL), was added PCl₃ (0.22 mL, 2.5622 mmol) at 25-30° C. After complete addition, the reaction mixture was heated to 60±5° C. for 60 hours. The reaction completion was monitored by LC-MS. Upon reaction completion, the reaction mixture was quenched with excess of water (25 mL) and extracted with DCM (10 mL). The organic phase was dried on anhydrous sodium sulphate and concentrated under reduced pressure to get crude compound. The crude compound was purified by preparative-HPLC. The combined pure fraction was concentrated and dried under high vacuum to get VMY-TP6 as beige solid (70 mg, 17.8%). HRMS (ESI): exact mass calculated for C₁₂H₈F₂N₆O₂ [M+H]⁺, 307.07, found 307.6 ¹H NMR (400 MHz, DMSO-d₆) δ: 10.86 (s, 1H), 9.00 (s, 1H), 7.74-7.69 (m, 2H), 7.01-6.69 (m, 1H), 3.89 (s 1H).

### Example 7

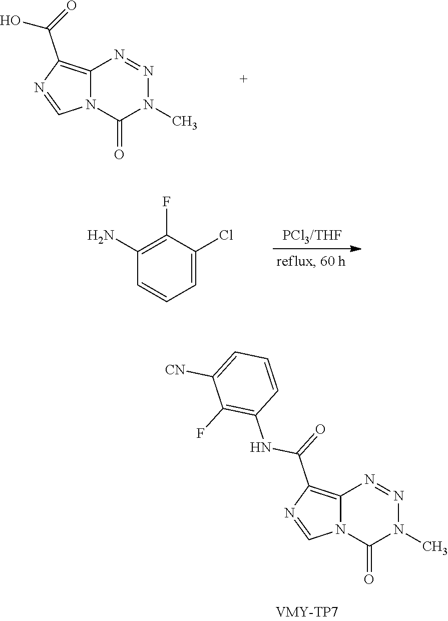

N-(3-chloro-2-fluorophenyl)-3-methyl-4-oxo-3,4-dihydroimidazo[5,1-d][1,2,3,5]tetrazine-8-carboxamide (VMY-TP7): To a stirred solution of temozolomide acid (80 mg, 0.0004 mmol.) in THF (0.8 mL, 10 vol), was added 3-chloro-2-fluoroaniline (71 mg, 0.0005 mmol) followed by PCl3 (112 mg, 0.0008 mmol.) and cooled to 25° C. To the reaction mass, was heated to reflux for 60 hours. The reaction completion was monitored by LC-MS. Upon reaction completion, the reaction mixture was diluted with EA (0.8 mL, 10 vol) and quenched with water (1.6 mL, 20 vol). The layers were separated and the organic layer was washed with sat. sodium chloride solution and separated layers. The organic layer was concentrated to 5.0 vol and filtered. The resulted solid was dried under reduced pressure to get VMY-TP7 as pale yellow solid (70 mg, 53%). HRMS (ESI): exact mass calculate for C₁₂H₈ClFN₆O [M+H]⁺, 324.04, found 324.3. ¹H NMR (400 MHz, DMSO-d₆) δ: 10.22 (s, 1H), 8.98 (s, 1H), 7.85-7.18 (m, 1H), 7.47-7.43 (m, 1H), 7.30-7.25 (m, 1H), 3.90 (s 3H).

### Example 8

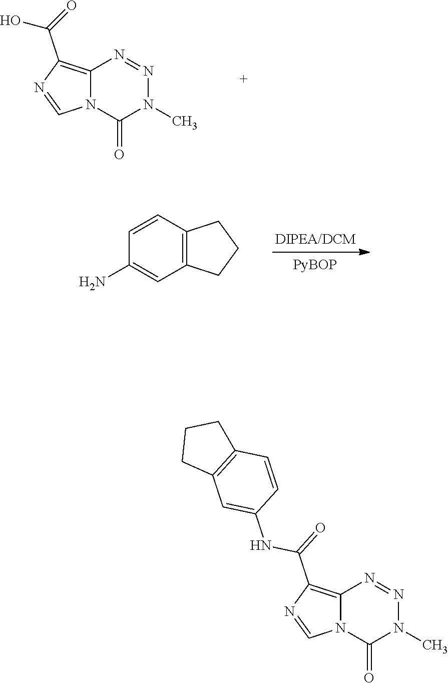 [SMILES: Cn1nnc2c(C(=O)O)ncn2c1=O ; Nc1ccc2c(c1)CCC2 ; Cn1nnc2c(C(=O)Nc3ccc4c(c3)CCC4)ncn2c1=O]

N-(2,3-dihydro-1H-inden-5-yl)-3-methyl-4-oxo-3,4-dihydroimidazo[5,1-d][1,2,3,5]tetrazine-8-carboxamide (VMY-TP8): To a stirred solution of temozolomide acid (160 mg, 0.8199 mmol.) in DCM (3.2 mL), was added DIPEA (0.42 mL, 2.4597 mmol.) and cooled to 0-5° C. To the reaction mass, was added Py-Bop (512 mg, 0.9839 mmol.) and allowed to stir at 25-30° C. for 1 hour. The reaction completion was monitored by LC-MS. Upon reaction completion, the reaction mixture was diluted with DCM (16 mL) and quenched with water (4 mL). The layers were separated and the aqueous layer was further extracted with DCM (16 mL). The combined organic layer was dried over anhydrous sodium sulphate and concentrated under reduced pressure to get crude compound. The crude compound was suspended in acetonitrile (5 mL) and stirred for 30 minutes at 25-30° C. Resulted solid was collected by filtration and washing with acetonitrile. Obtained pure compound was dried under high vacuum to get VMY-TP8 as pale yellow solid (80 mg, 31.5%). HRMS (ESI): exact mass calculated for C₁₂H₈ClFN₆O [M+H]⁺, 311.12, found 311.7. ¹H NMR (400 MHz, DMSO-d₆) δ: 10.25 (s, 1H), 8.95 (s, 1H), 7.77 (s, 1H), 7.57 (d, J=8 Hz, 1H), 7.19 (d, J=8 Hz, 1H), 3.88 (s, 3H), 2.88-2.81 (m, 4H), 2.04 (t, J=7.6 Hz, 2H).

### Example 9

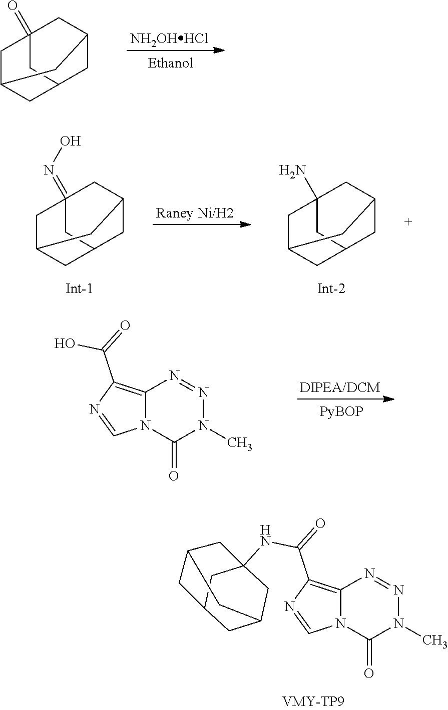

(1λ⁵-adamantan-1-ylidene)(hydroxy)azane (Int-1): To a stirred solution of adamantane-2-one (2 g, 0.0133 mol.) in hot (75±5° C.) ethanol (12 mL), was added a solution of hydroxylamine hydrochloride (2.13 g, 0.0306 mol.) in 2N aqueous sodium hydroxide (10 mL) and stirred for 1 hour. The completion of the reaction was monitored through TLC. After reaction completion, the reaction mixture was concentrated under vacuum, diluted with water and filtered. The solid filtered was washed with water and ethanol. The compound was dried under high vacuum to afford Int-1 as white solid (2 g, 90.0%). ¹H NMR (400 MHz, CDCl₃) δ: 3.58 (s, 1H), 2.59 (s, 1H), 2.00-1.99 (m, 2H), 1.95 (s, 2H), 1.92 (s, 1H), 1.88-1.82 (m, 6H).

Adamantan-1-amine (Int-2): Int-1 (1.8 g, 0.0108 mol.) and ethanol (9 mL, 5 vol.) were charged in autoclave and was added Raney nickel (0.18 g, 50% Wt./Wt.) under Argon atmosphere. The solution was degassed with argon thrice and pressurized with 3 Kg/Cm² of H₂ gas. Reaction mass was stirred for 3 hours. The reaction completion was monitored by TLC. After reaction completion, the reaction mixture was filtered through celite bed. The filtrate was concentrated and dried under high vacuum to afford Int-2 as white solid (1.3 g, 64.0%). ¹H NMR (400 MHz, DMSO-d₆) δ: 8.26 (brs, 3H), 3.27 (brs, 1H), 2.04-1.99 (m, 4H), 1.82-1.68 (m, 8H), 1.53 (d, J=12.4 Hz, 2H).

N-(adamantan-1-yl)-3-methyl-4-oxo-3,4-dihydroimidazo[5,1-d][1,2,3,5]tetrazine-8-carboxamide (VMY-TP9): To a stirred solution of temozolomide acid (120 mg, 0.7687 mmol.) in DCM (3 mL), was added DIPEA (0.4 mL, 2.3061 mmol.) and cooled to 0-5° C. To that, was added Py-Bop (480 mg, 0.9224 mmol.) and stirring continued for 1 hour at 25-30° C. The reaction completion was monitored by LC-MS. After reaction completion, the reaction mixture was quenched with water (10 mL) and phases were separated. The aqueous phase was further extracted with DCM (5 mL). The combined organic phase was dried over anhydrous sodium sulphate and concentrated under reduced pressure to get crude compound. The crude compound was purified through preparative-HPLC. The combined pure fraction was concentrated and dried under high vacuum to get VMY-TP4 as pale yellow solid (70 mg, 41%). HRMS (ESI): exact mass calculated for C₁₆H₂₀N₆O₂ [M+H]⁺, 329.16, found 329.7 ¹H NMR (400 MHz, DMSO-d₆) δ: 8.85 (s, 1H), 7.92 (d, J=8 Hz, 1H), 4.12 (d, J=7.6 Hz, 1H), 3.87 (s, 3H), 1.97 (s, 2H), 1.92-1.85 (m, 8H), 1.73 (s, 2H), 1.65-1.62 (brd, J=12.4 Hz, 2H).

### Example 10

### Physicochemical Properties of Selected Compounds

Physicochemical properties of selected compounds were determined with respect to CNS permeability (Table 1). The synthesis of TP3, TP5, and TP9 are described in Examples 3, 5, and 9, respectively. MPO values are 0-6, with 6 being most desirable and MPO 4-6 consistent with passive CNS permeability.

Log BB=0.152 C log P−0.0148 PSA+0.130

TABLE 1
Comparison of Physicochemical properties
with guidelines for CNS permeability

| Compound | Mw | Log P | TPSA (A²) | cLOGBB | MPOₘₐₓ |
|---|---|---|---|---|---|
| TMZ | 194.15 | −0.84 | 108 | −1.52 | 3 |
| TP3 | 278.32 | 1.20 | 94 | −0.86 | 4 |
| TP5 | 276.30 | 0.80 | 94 | −0.95 | 4 |
| TP9 | 328.38 | 1.54 | 90 | −0.86 | 5 |

### Example 11

### Activity of Selected Compounds in Glioma Cells

The effect of selected compounds on glioma cells viability was determined. The cells were plated at 2000 cells/well in 96 well plates. The day after, cells were treated with various concentrations of temozolomide or VMY-TP analogs. Cell viability was measured by AlamarBlue™ cell viability reagent after 72 hours, considering the control level of cells to be 100%, and IC₅₀ was calculated (Table 2). The compounds exhibited greater potency in standard cell lines (U251, U87, and T98G) and a patient-derived cell line (GBM8) compared to TMZ.

TABLE 2
Effect of compounds on glioma cell viability

| | |
|---|---|
| MGMT | IC₅₀ (μmol/L), 72 h |

| Cell Line | Expression | TMZ | TP3 | TP5 | TP9 |
|---|---|---|---|---|---|
| U251 | − | 898 | 397 | 139 | 326 |
| U87 | − | 208 | 399 | 168 | 99 |
| GBM8 | − | 45 | 2 | 28 | 16 |
| T98G | + | 1145 | 1257 | 581 | 408 |

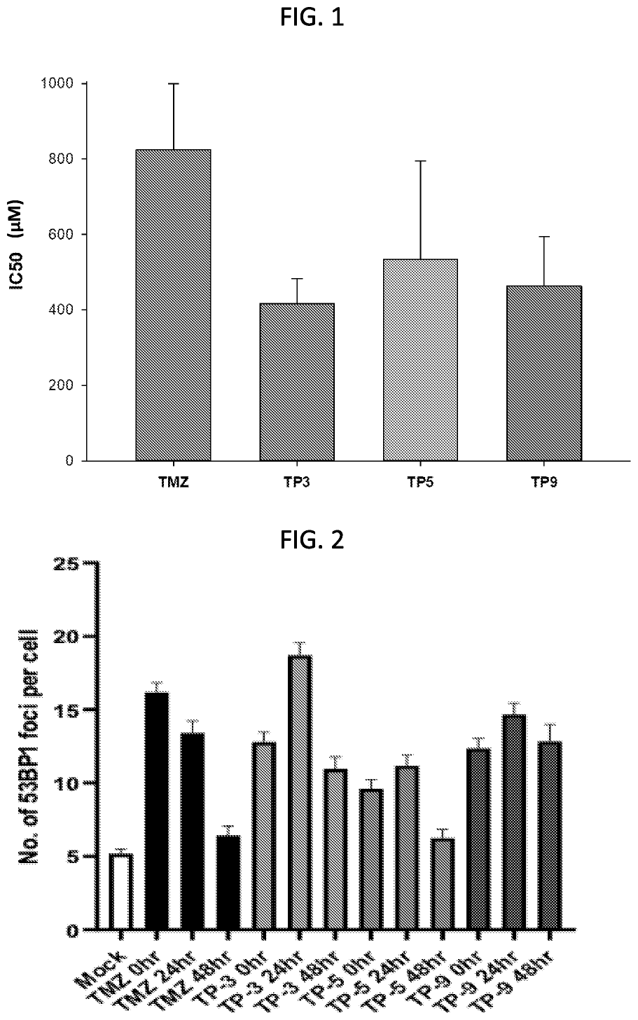

Cell proliferation was also investigated in the U87 cell line. U87 cells were seeded at the density of 2,000 cells/well in 96-well plates and incubated overnight. The day after, cells were treated with various concentrations of Temozolomide or analogs for 72 hours. The BrdU ELISA proliferation assay was performed according to the manufacturer's instructions (Roche, #11647229001). Briefly, 20 μl of diluted BrdU labelling reagent was added to each well, to a final concentration of 10 μM BrdU per well. The cells were then incubated at 37° C. for 24 hours. Following this incubation, the labeling medium was aspirated from each well and 200 μl of Roche FixDenat fixing buffer was added. The plates were then incubated at 25° C. for 30 minutes. The fixing solution was removed, and 100 μl of Anti-BrdU-POD antibody was added to each well and incubated again at 25° C. for 90 minutes. Wells were rinsed three times with 200 μl of PBS, then 100 μl of Roche Substrate solution was added to each well. Following a 25 minute incubation at room temperature, the absorbance of each plate was read at 370 nm (reference wavelength: approx. 492 nm) on a Spectramax® i3x microplate reader. The proliferation rate was evaluated by measuring the incorporation of BrdU (FIG. 1).

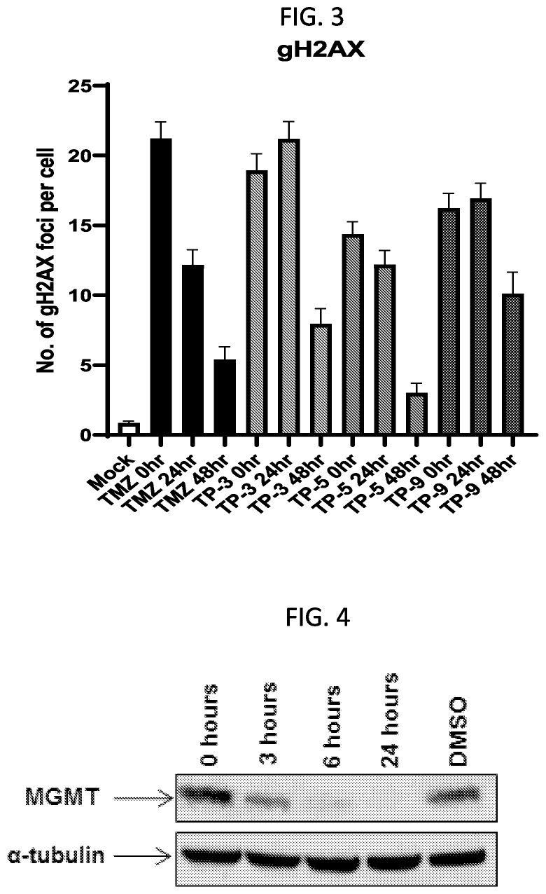

Expression of 53BP1 foci, a marker for double strand break formation and DNA damage, was analyzed in U251 cells treated with 10 μM of each test compound for 48 hours and immuno-stained with antibodies to 53BP1. Rates of double-strand break repair were determined by scoring 53BP1 foci and plotting the average number of 53BP1 foci per cell. Each of the tested compounds had similar activity to TMZ (FIG. 2). Phosphorylation of the Ser-139 residue of the histone variam H2AX, forming γH2AX, is an early cellular response to the induction of DNA double-strand breaks. Detection of this phosphorylation event has emerged as a highly specific and sensitive molecular marker for monitoring DNA damage initiation and resolution. γH2AX was measured in U251 cells treated with TMZ or test compounds (FIG. 3).

T98G cells, which express MGMT, were treated with TMZ or test compounds with or without 3, 6, or 24 hours of pretreatment with 100 μM of O⁶-benzylguanine (O⁶-BG; a pseudo-substrate of MGMT that quenches the cellular store of enzyme). T98G cell lysates were probed with anti-MGMT antibody and then with anti-a tubulin (FIG. 4). In a separate experiment, TMZ or test compounds were added to T98G cells with or without 5-6 hours of pretreatment with 100 μM O⁶-BG. Cell viability was measured using the Cell Titer Glo assay after 3 days of incubation and IC₅₀ values were determined (Table 3). These data demonstrate that TP3, TP5, and TP9 undergo similar alkylation mechanism compared to TMZ (e.g., less potency in presence of MGMT and more potency in absence of MGMT). These analogs also showed potency in MGMT expressing cell line.

TABLE 3
T98G cell viability with or without O6-BG pretreatment

IC50 (μM)

| | With O⁶-BG | Without O⁶-BG |
|---|---|---|
| Compound | pretreatment | pretreatment |
| TMZ | >1000 | >1000 |
| TP3 | 675 | 1050 |
| TP5 | 168 | 410 |
| TP9 | 148 | 512 |
| VAL-083 (DelMar | 71 | 108 |
| Pharmaceuticals) | | |

### Example 12

### Stability of Selected Compounds

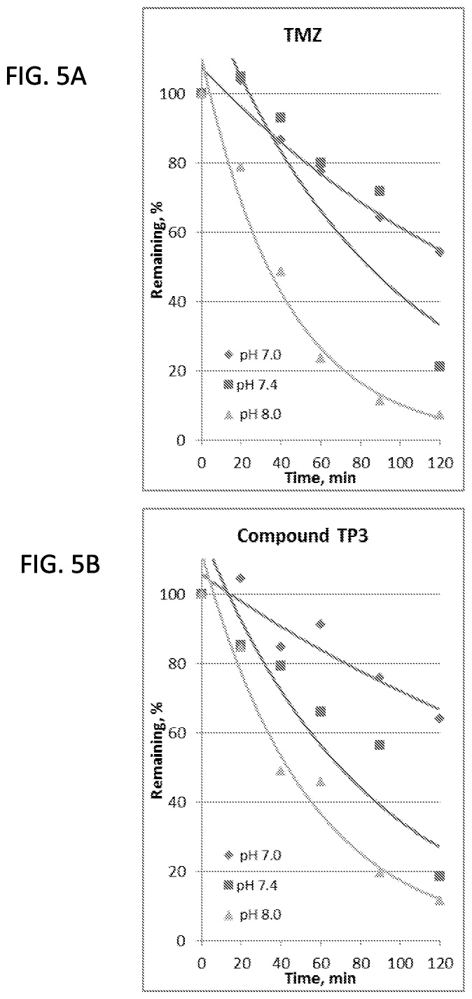

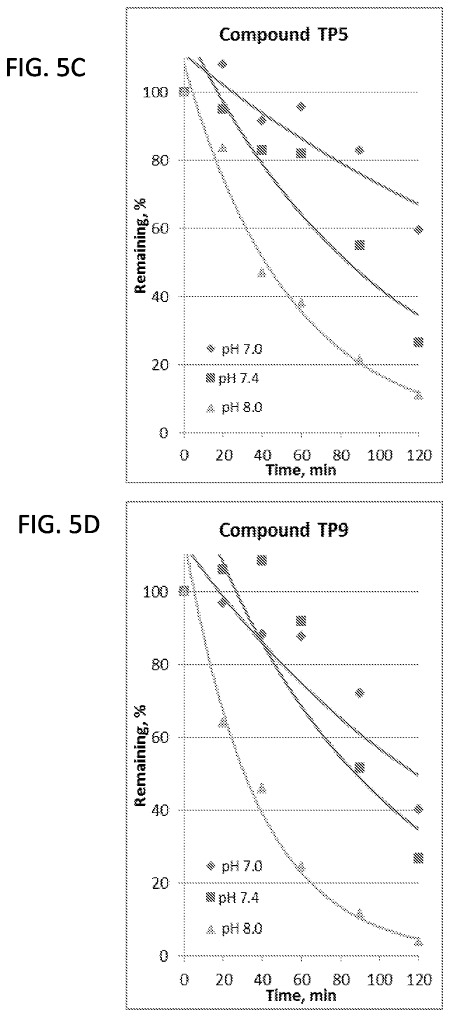

Short-term stability (0-2 hours) of TMZ, TP3, TP5, and TP9 (1 μM) was determined in aqueous media (0.1 M phosphate buffer (pH 7.0), PBS (pH 7.4), 0.1 M phosphate buffer (pH 8.0)) using HPLC-MS. Detection of products of non-enzymatic cleavage (AIC-TMZ, AIC-TP3, AIC-TP5, and AIC-TP9) was performed (Table 4; FIGS. 5A-5D). Chemical stability of all compounds decreased with increasing pH. The lowest stability was in 0.1 M phosphate buffer, pH 8.0.

TABLE 4
Aqueous chemical stability of selected compounds

| | t_{1/2} (min) (0-2 h) | | |
|---|---|---|---|
| Compound | pH 7.0 | pH 7.4 | pH 8.0 |
| TMZ | 119 | 59 | 31 |
| TP3 | 119 | 62 | 39 |
| TP5 | >120 | 69 | 40 |
| TP9 | 103 | 59 | 29 |

Metabolic stability of the compounds was also determined in mouse liver microsomes at five time points over 60 minutes, using HPLC-MS. Liver microsomes, as vesicles of the hepatocyte endoplasmic reticulum, contain membrane phase I enzymes namely CYPs, flavine-containing monooxygenases (FMO), esterases, amidases, and epoxide hydrolases, and also the phase II enzymes such as UGTs. For the catalytic activity of both phase I and II enzymes, addition of exogenous cofactors such as NADPH for CYPs and FMO, and UDPGA/alamethicin for UGTs is necessary. Metabolites AIC-TMZ, AIC-TP3, AIC-TP, and AIC-TP9 were detected (Table 5 and FIGS. 5A-5D). Metabolic stability is defined as the percentage of parent compound lost over time in the presence of a metabolically active test system:

$${Cl}_{int} = \frac{0.693}{{t}_{1 / 2}} \times \frac{μ {l}_{incubation}}{{mg}_{microsomes}}$$

TABLE 5
Metabolic stability of selected compounds

| | | | Intrinsic | Clearance |
|---|---|---|---|---|
| | % remaining (1 h) | | clearance | Classification |
| | With | Without | (Clint) | (Low <8.6; |
| Compound | co-factors | co-factors | (μl/min/mg) | High >48) |
| TMZ | 65 | 72 | 15 | Moderate |
| TP3 | 0 | 80 | 358 | High |
| TP5 | 49 | 94 | 25 | Moderate |
| TP9 | 2 | 99 | 157 | High |
| Propranolol | 19 | 88 | 104 | High |

### Example 13

### Permeability of Selected Compounds

Permeability of the compounds was evaluated in the bidirectional Caco-2 assay including identification of P-glycoprotein substrate (Pgp-mediated transport). Verapamil was used as a p-gp inhibitor. The apparent permeability (Papp) was calculated using the following equation:

Papp=VA/Area×Time×[drug]acc/[drug]initial,d

The compounds exhibited high permeability in A-B direction and did not undergo active efflux (Table 6). In addition, the compounds were not Pgp substrates.

TABLE 6
Permeability of compounds in Cac-2 A-B/B-A assay

| | A-B/B-A permeability | | | | Efflux Ratio | |
|---|---|---|---|---|---|---|
| | Pₐₚₚ (AB), 10⁻⁶ cm/s | | Pₐₚₚ (BA), 10⁻⁶ cm/s | | BA/AB | BA/AB |
| | p-gp | p-gp | p-gp | p-gp | p-gp | p-gp |
| Com- | Inhibitor | Inhibitor | Inhibitor | Inhibitor | Inhibitor | Inhibitor |
| pound | (−) | (+) | (−) | (+) | (−) | (+) |
| TMZ | 20.2 | 18.1 | 21.0 | 17.2 | 1.0 | 0.9 |
| TP3 | 30.7 | 18.6 | 17.0 | 13.2 | 0.6 | 0.7 |
| TP5 | 26.6 | 25.7 | 24.3 | 19.4 | 0.9 | 0.8 |
| TP9 | 28.3 | 28.0 | 19.5 | 16.7 | 0.7 | 0.6 |
| Dig- | 1.2 | 5.9 | 15.8 | 6.1 | 13.6 | 1.0 |
| oxin | | | | | | |

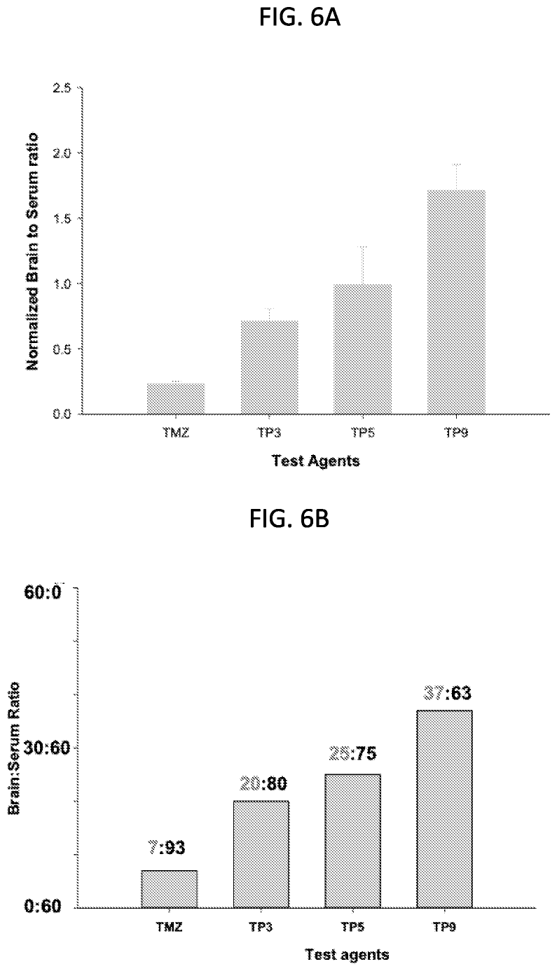

BBB permeability of the compounds was tested in male CD-1@IGS mice using cassette intravenous dosing (Table 7 and FIGS. 6A and 6B). There were three animals in each group, given a dose of 5 mg/kg formulated in 10% DMSO in PBS (v/v) for 5 minutes. The endpoint was quantifying the parent compound using LC-MS/MS. Plasma samples (50 μl) were immediately mixed with 25 μl of H₃PO₄ (8.5%). Then 225 μl of internal standard (IS; 400 ng/ml meldonium in methanol) was added. After mixing by pipetting and centrifuging for 4 min at 6000 rpm, 2 μl of each supernatant was injected into LC-MS/MS system. Brain samples (100 mg±1 mg) were immediately mixed with 100 μl H₃PO₄ (25%) and zirconium oxide beads (115 mg±5 mg). Then 400 μl of IS solution was added and samples were disperse using a bullet blender homogenizer for 30 seconds at speed 8. The samples were centrifuged for 4 min at 14,000 rpm and 2 μl of each supernatant was injected into LC-MS/MS system.

TABLE 7
BBB permeability of test compounds following
cassette intravenous dosing

| | Plasma | Brain | | | | |
|---|---|---|---|---|---|---|
| | Conc | Conc. | Brain/ | % | % | |
| Compound | (ng/ml) | (ng/g) | Plasma | Brain | Plasma | Brain:Plasma |
| TMZ | 5537 | 1287 | 0.23 | 7 | 93 | 7:93 |
| TP3 | 2920 | 2080 | 0.71 | 20 | 80 | 20:80 |
| TP5 | 3116 | 3052 | 0.99 | 25 | 75 | 25:75 |
| TP9 | 1830 | 3122 | 1.71 | 37 | 63 | 37:63 |

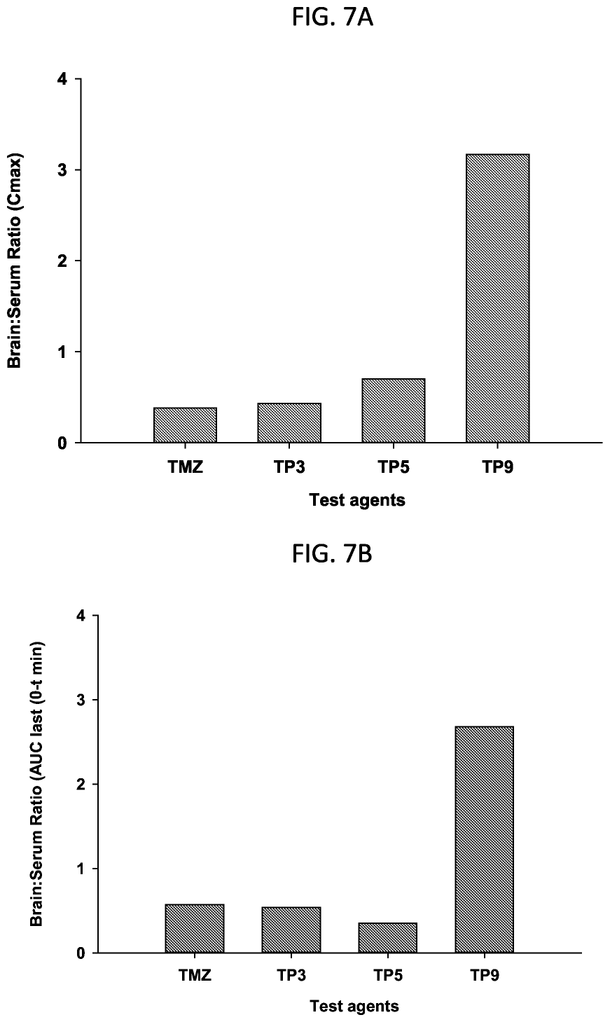

BBB permeability of the compounds was also tested in male CD-1@IGS mice using cassette oral dosing (Table 8 and FIGS. 7A and 7B3). Representative selected pharmacokinetics (Cₘₐₓ, T_{1/2}, and AUC) of TMZ and selected compounds were measured over time (0-24 h) in CD-i mice following cassette peroral (PO) dosing. Detection methods were as for the intravenous dosing study except that brain samples were not dilated with 100 μl H₃PO₄.

TABLE 8
Comparison of selected pharmacokinetic
parameters in oral dosing study

| | | Plasma | Brain | |
|---|---|---|---|---|
| | | (Parent + | (Parent + | Brain/ |
| Compound | Parameter | AIC) | AIC) | Plasma |
| TMZ | Cₘₐₓ (ng/mL) | 2270 | 877 | 0.38 |
| | T_{1/2} (min) | 145 | 115 | |
| | AUC₀₋ₜ (min*ng/mL) | 201400 | 115000 | 0.57 |
| TP3 | Cₘₐₓ (ng/mL) | 668 | 287 | 0.42 |
| | T_{1/2} (min) | 31 | 9 | |
| | AUC₀₋ₜ (min*ng/mL) | 6660 | 3610 | 0.54 |
| TP5 | Cₘₐₓ (ng/ml) | 2502 | 1756 | 0.70 |
| | T_{1/2} (min) | 124 | 51 | |
| | AUC₀₋ₜ (min*ng/ml) | 100200 | 35678 | 0.35 |
| TP9 | Cₘₐₓ (ng/ml) | 139 | 441 | 3.17 |
| | T_{1/2} (min) | 197 | 15 | |
| | AUC₀₋ₜ (min*ng/ml) | 3280 | 8820 | 2.68 |

### Example 14

### Activity of Compounds in TMZ Resistant Cell Lines

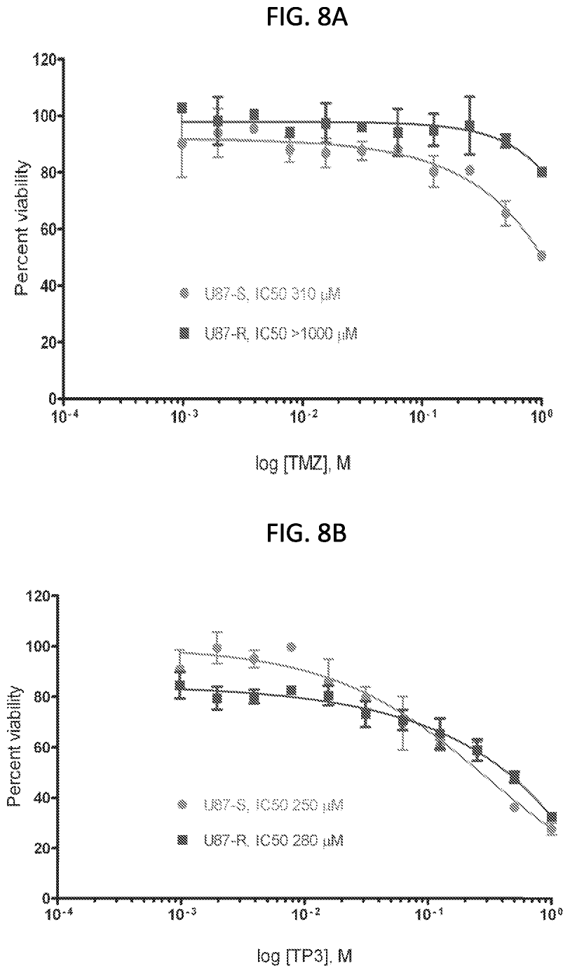

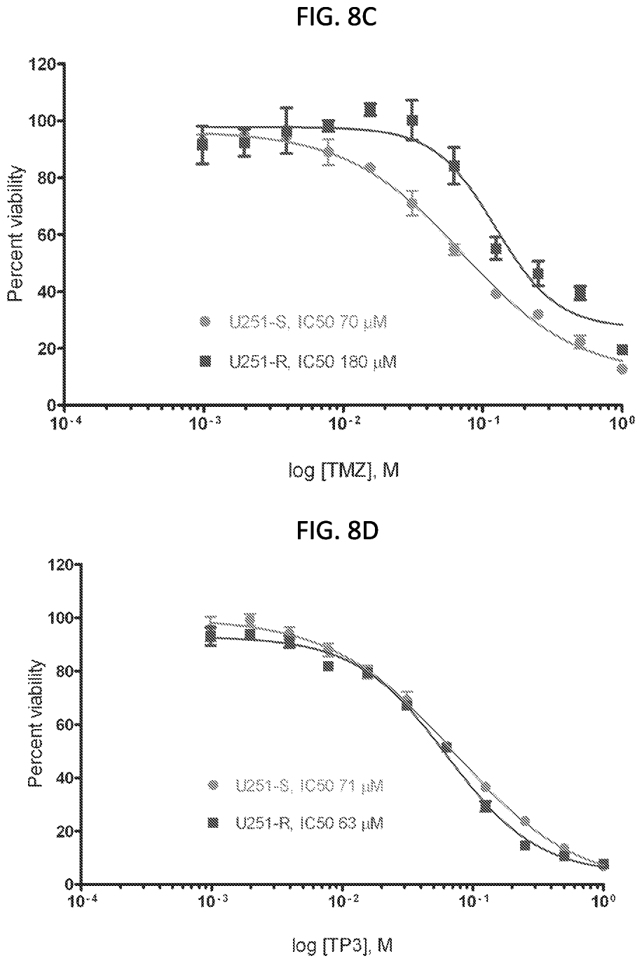

Cells were seeded in 96-well plates. The day after, cells were treated with various concentrations of temozolomide or TP3. Cell viability was measured by AlamarBlue™ cell viability reagent after 72 hours, considering the control level of cells to be 100%. While cells were resistant to TMZ, they were not resistant to TP3 (FIGS. 8A-8D and Table 9).

TABLE 9
Cell viability in TMZ sensitive and resistant cell lines

| | |
|---|---|
| Parent (Sensitive) | Resistant |

U87MG (IC₅₀, μM)

| | | |
|---|---|---|
| TMZ | 310 | >1000 |
| TP3 | 250 | 280 |

U251 (IC₅₀, μM)

| | | |
|---|---|---|
| TMZ | 70 | 180 |
| TP3 | 71 | 63 |

### Example 15

### Formulation Stability and Kinetic Studies of TP3

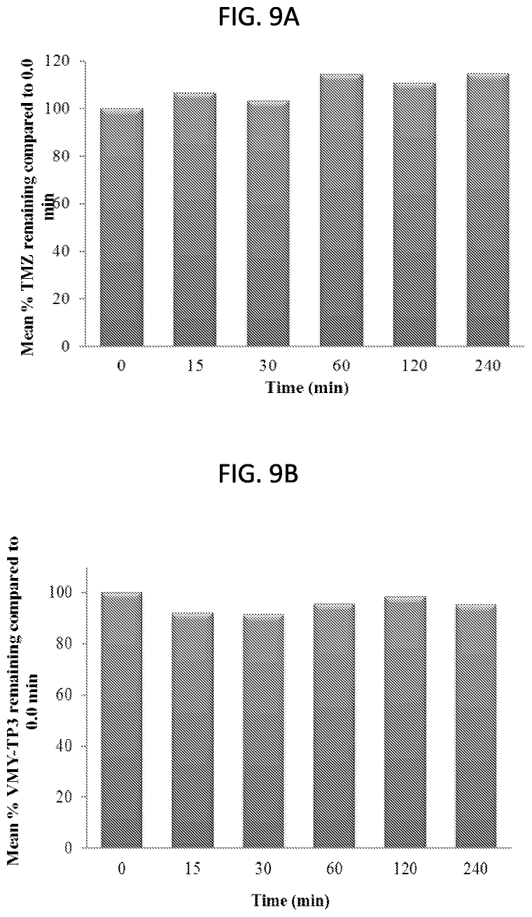

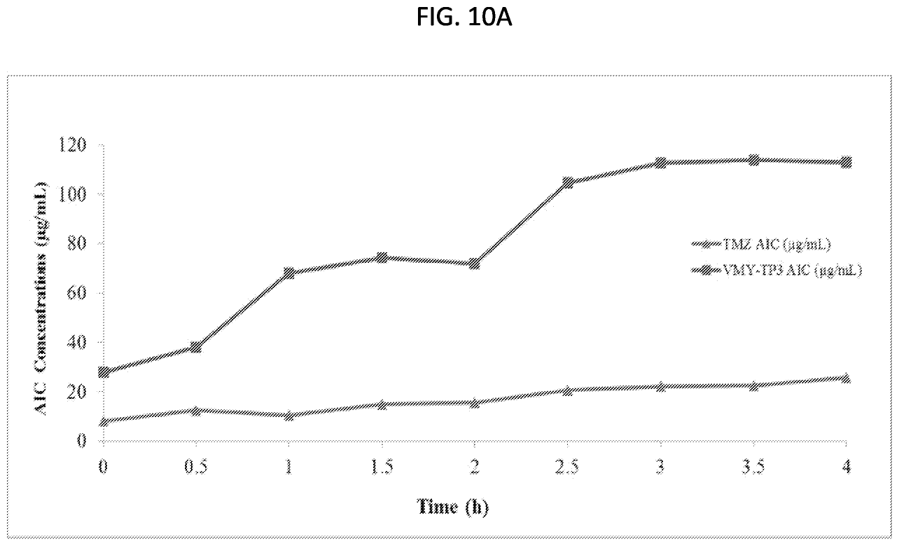

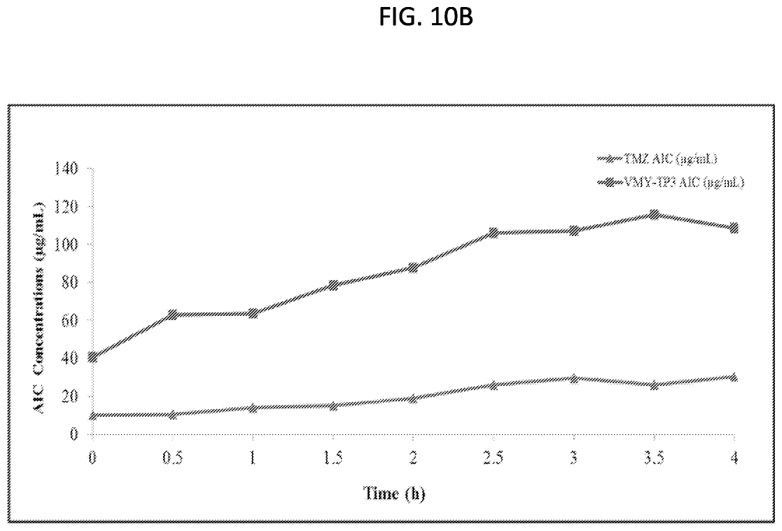

Two formulations for TP3 and TMZ were prepared (Tables 10 and 11). Pre-formulation testing was in volumes of 100 μl-250 μl, and should be confirmed with larger volumes. Mice were dosed at 20 mg/kg of TMZ and TP3 using formulation 2 orally and brain and plasma samples were collected at 0, 0.5, 1, 3, 6 and 12 h timepoints and the concentration was measured using LC-MS and calculate PK parameters using standard equation. The stability profile (FIGS. 9A and 9B) and release kinetics profiles (FIGS. 10A and 10B) were compared. Single dose oral brain to plasma pharmacokinetic parameters were determined with Formulation 2 (Table 12).

TABLE 10
TMZ and TP3 formulations

| | Com- | | | Conc. | | Stability |
|---|---|---|---|---|---|---|
| ID | pound | Vehicle | pH | (mg/ml) | Remarks | (0-4 h) |
| Formu- | TP3 | 0.4% (v/v) Tween 80 + | 4.81 | 2 | Clear | Stable |
| lation | | 2% (v/v) glycerol + | | | solution | |
| 2 | | 97.6% (v/v) of 30% | | | | |
| | | (w/v) Captisol (SβECD) | | | | |
| Formu- | TP3 | 40% (v/v) Labrasol + | 5.66 | 2 | Clear | Stable |
| lation | | 40% (v/v) Labrafil + | | | solution | |
| 3 | | 20% (v/v) Transcutol | | | | |
| Formu- | TMZ | 0.4% (v/v) Tween 80 + | 4.77 | 2 | Clear | Stable |
| lation | | 2% (v/v) glycerol + | | | solution | |
| 2 | | 97.6% (v/v) of 30% | | | | |
| | | (w/v) Captisol (SβECD) | | | | |
| Formu- | TMZ | 40% (v/v) Labrasol + | 6.01 | 2 | Clear | Stable |
| lation | | 40% (v/v) Labrafil + | | | solution | |
| 3 | | 20% (v/v) Transcutol | | | | |

TABLE 12
Single dose oral brain to plasma PK parameters

| | | | | Brain/ |
|---|---|---|---|---|
| | Parameter | Plasma (AIC) | Brain (AIC) | Plasma |
| TMZ | Cₘₐₓ (ng/mL) | 7452 | 2787 | 0.37 |
| | T_{1/2} (h) | 1.26 | 2.26 | |
| | AUC₀₋ₜ (min*ng/mL) | 15326 | 8675 | 0.56 |
| TP3 | Cₘₐₓ (ng/ml) | 601 | 399 | 0.66 |
| | T_{1/2} (h) | ND | ND | |
| | AUC₀₋ₜ (min*ng/mL) | 364 | 252 | 0.69 |

TABLE 11
Additional TMZ and TP3 formulations

| | | | Concentration | | | |
|---|---|---|---|---|---|---|
| Compound | Vehicle Composition | % of Vehicles used | (mg/mL) | Remarks | Comments | pH |
| TP3 | 62.5 μL of DMSO + 187.5 μL | 25% of DMSO + 75% Normal | 4.00 | Not Soluble | | NA |
| | Normal Saline | Saline | | | | |
| TP3 | 25 μL DMA + 12.5 μL Tween | 10% DMA + 5% Tween 80 + | 4.0 | Not Soluble | | NA |
| | 80 + 212.5 μL Normal saline | 85% Normal saline | | | | |
| TP3 | 25 μL DMA + 25 μL Tween | 10% DMA + 10% Tween 80 + | 4.0 | Not Soluble | | NA |
| | 80 + 25 μL PEG 400 + 25 μL | 10% PEG 400 + 10% PG + | | | | |
| | PG + 150 μL Normal saline | 60% Normal saline | | | | |
| TP3 | 10 μL DMA (20 mg/mL DMA | 10% DMA + 10% Tween 80 + | 2.0 | Not Soluble | | NA |
| | stock) + 10 μL Tween 80 + | 10% PEG 400 + 10% PG + | | | | |
| | 10 μL PEG 400 + 10 μL PG + | 60% Normal saline | | | | |
| | 60 μL Normal saline | | | | | |
| TP3 | 10 μL DMA (20 mg/mL DMA | 10% DMA + 10% Tween 80 + | 2.0 | Soluble | | ~6 |
| | stock) + 10 μL Tween 80 + | 15% PEG 400 + 15% PG + | | | | |
| | 15 μL PEG 400 + 15 μL PG + | 50% of 15% (w/v) Captisol | | | | |
| | 50 μL of 15% (w/v) Captisol | | | | | |
| TP3 | 10 μL DMA (20 mg/mL DMA | 10% DMA + 10% Tween 80 + | 2.0 | Soluble | | ~6 |
| | stock) + 10 μL Tween 80 + | 10% PEG 400 + 10% PG + | | | | |
| | 10 μL PEG 400 + 10 μL PG + | 60% of 15% (w/v) Captisol | | | | |
| | 60 μL of 15% (w/v) Captisol | | | | | |
| TP3 | 10 μL DMA (20 mg/mL DMA | 10% DMA + 10% Tween 80 + | 2.0 | Soluble | | ~6 |
| | stock) + 10 μL Tween 80 + | 15% PEG 400 + 15% PG + | | | | |
| | 15 μL PEG 400 + 15 μL PG + | 50% of 25% (w/v) Captisol | | | | |
| | 50 μL of 25% (w/v) Captisol | | | | | |
| TP3 | 25 μL DMA + 12.5 μL Tween | 10% DMA + 5% Tween 80 + | 4.0 | Not Soluble | | NA |
| | 80 + 37.5 μL PEG 400 + | 15% PEG 400 + 15% PG + | | | | |
| | 37.5 μL PG + 137.5 μL of 25% | 50% of 25% (w/v) Captisol | | | | |
| | (w/v) Captisol | | | | | |
| TP3 | 25 μL DMA + 25 μL Tween | 10% DMA + 10% Tween 80 + | 4.0 | Soluble | Formulation was clear | ~6 |
| | 80 + 25 μL PEG 400 + 25 μL | 10% PEG 400 + 10% PG + | | | initially, but precipitated | |
| | PG + 150 μL of 50% (w/v) | 60% of 50% (w/v) Captisol | | | with time (after 2 h) | |
| | Captisol | | | | | |
| TP3 | 12.5 μL DMA + 25 μL Tween | 5% DMA + 10% Tween 80 + | 4.0 | Soluble | Formulation was clear | ~6 |
| | 80 + 25 μL PEG 400 + 25 μL | 10% PEG 400 + 10% PG + | | | initially, but precipitated | |
| | PG + 162.5 μL of 50% (w/v) | 65% of 50% (w/v) Captisol | | | with time (after 2 h) | |
| | Captisol | | | | | |
| TP3 | 12.5 μL DMA + 25 μL Tween | 5% DMA + 10% Tween 80 + | 4.0 | Not Soluble | | NA |
| | 80 + 25 μL PEG 400 + 25 μL | 10% PEG 400 + 10% PG + | | | | |
| | PG + 162.5 μL of 50% (w/v) | 65% of 50% (w/v) HPβCD | | | | |
| | HPβCD | | | | | |
| TP3 | 12.5 μL DMA + 25 μL Tween | 5% DMA + 10% Tween 80 + | 2.0 | Not Soluble | | NA |
| | 80 + 25 μL PEG 400 + 25 μL | 10% PEG 400 + 10% PG + | | | | |
| | PG + 162.5 μL 15% (w/v) | 65% of 15% (w/v) Captisol | | | | |
| | Captisol | | | | | |
| TP3 | 25 μL DMA + 25 μL Tween | 10% DMA + 10% Tween 80 + | 2.0 | Not Soluble | | NA |
| | 80+ 25 μL PEG 400 + 25 μL | 10% PEG 400 + 10% PG + | | | | |
| | PG + 150 μL 15% (w/v) | 60% of 15% (w/v) HPβCD | | | | |
| | HPβCD | | | | | |
| TP3 | 25 μL DMA + 25 μL Tween | 10% DMA + 10% Tween 80 + | 2.0 | Not Soluble | Formulation was clear | NA |
| | 80 + 25 μL PEG 400 + 25 μL | 10% PEG 400 + 10% PG + | | | initially, but precipitated | |
| | PG + 150 μL 15% (w/v) | 60% of 15% (w/v) Captisol | | | with time (after 2 h) | |
| | Captisol | | | | | |
| TP3 | 25 μL DMA + 37.5 μL Solutol | 10% DMA + 15% Solutol | 2.0 | Soluble | | NA |
| | HS15 + 25 μL PEG 400 + | HS15 + 10% PEG 400 + 10% | | | | |
| | 25 μL PG + 137.5 μL 15% | PG + 55% of 15% (w/v) | | | | |
| | (w/v) Captisol | Captisol | | | | |
| TMZ | 25 μL DMA + 37.5μL Solutol | 10% DMA + 15% Solutol | 2.0 | Soluble | | NA |
| | HS15 + 25 μL PEG 400 + | HS15 + 10% PEG 400 + 10% | | | | |
| | 25 μL PG + 137.5 μL 15% | PG + 55% of 15% (w/v) | | | | |
| | (w/v) Captisol | Captisol | | | | |

PEG 400 = polyethylene glycol 400;
PG = propylene glycol;
SWFI = sterile water for injection;
DMA = N,N-dimethylacetamide;
Captisol ® = β cyclodextrin sulfobutyl ethers, sodium salts.

### Example 16

### Efficacy of TP3 in Mouse Model of GBM

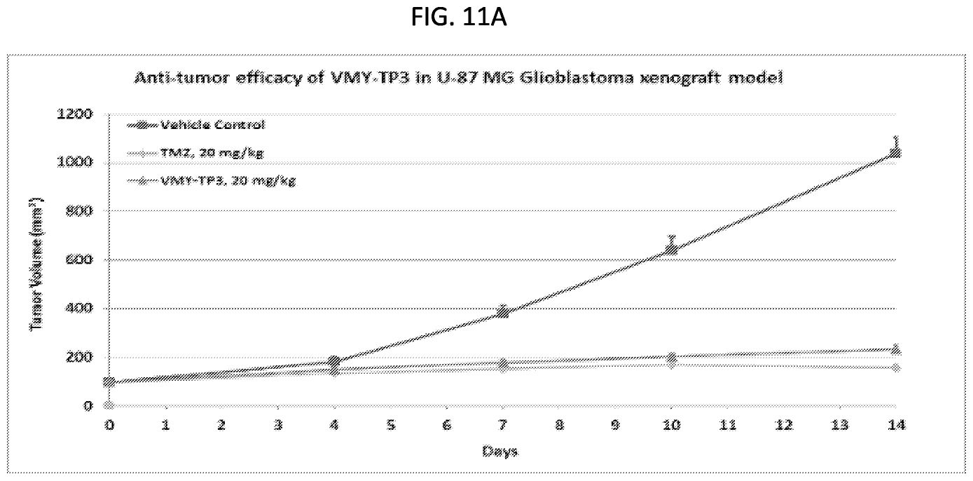

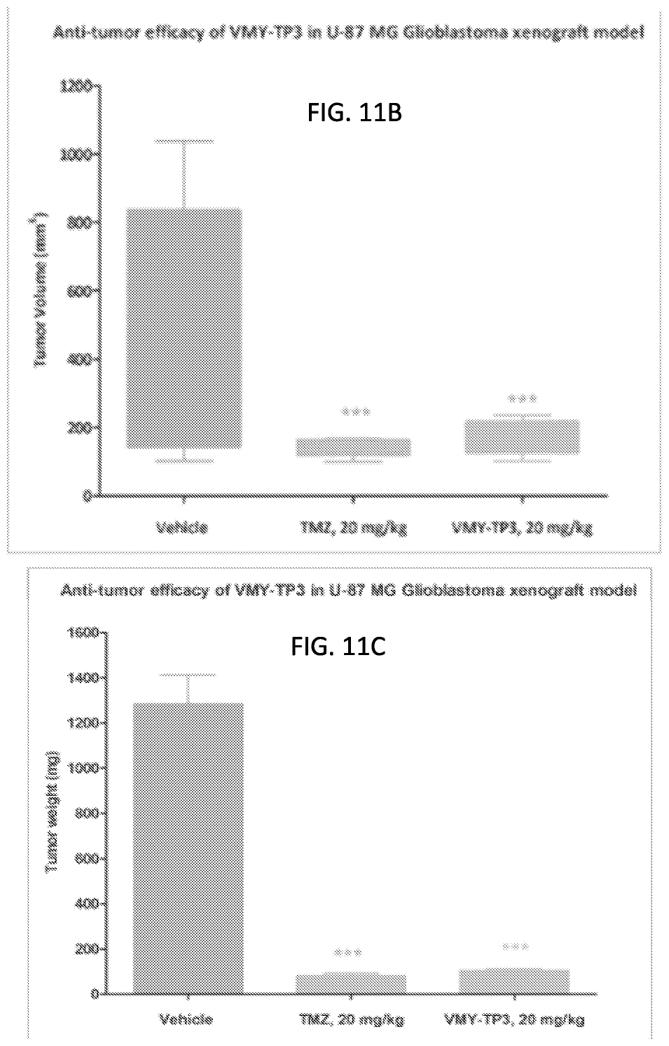

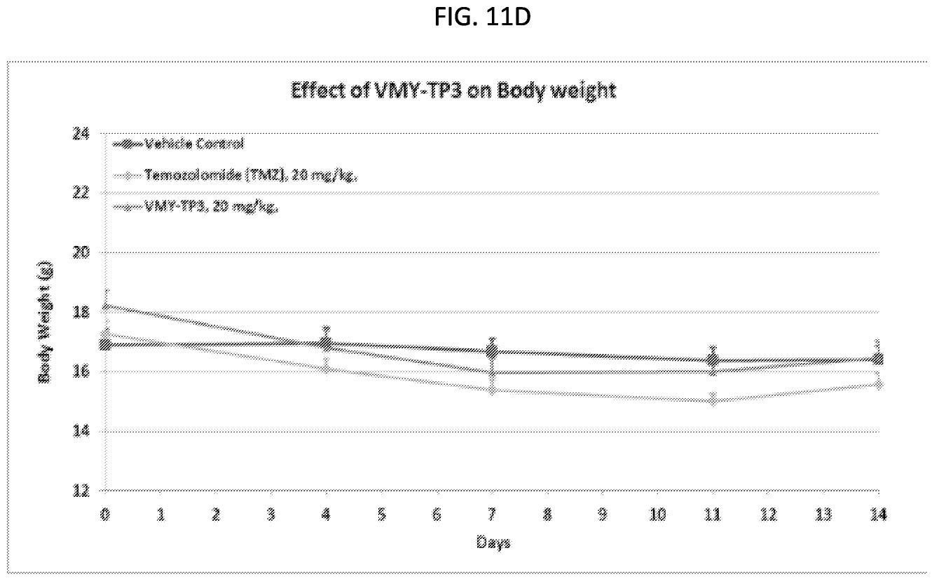

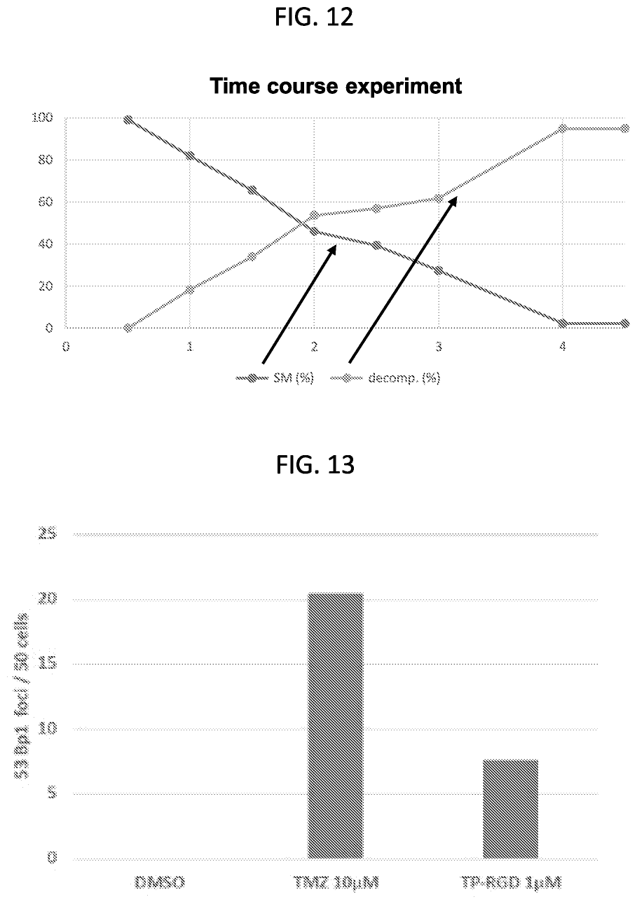

A suspension of U87 cells was prepared in 1×HBSS, pH 7.4 and mixed with an equal volume (1:1) of ice cold Matrigel®. Then, 0.1 mL of the cell suspension containing 5×10⁶ cells was injected subcutaneously using a 22-gauge needle into the flank region of SCID mice. Animals were monitored daily during the period between inoculation and study initiation. Treatment was started when the average tumor size reached approximately ˜100 mm³. The test (TP3) and Reference compound (TMZ) were administered orally at 20 mg/kg at a dose volume of 10 mL/Kg. The exact dose was administered based on individual animal body weight recorded during the study period. The TP3 and TMZ treatments were scheduled for once daily dosing for 14 days. However, the dose frequency had to be reduced from once daily to every other day, due to a 10-14% loss of body weight seen in the treated mice from Day 9 until study completion (FIG. 11D). The control group was administered vehicle alone. The test and reference compounds were formulated in 0.4% (v/v) Tween 80, 2% (v/v) Glycerol and 97.6% (v/v) of 30% (w/v) Captisol (SPECD). Number of mice per treatment cohort=8. Tumor growth was measured twice weekly using a digital Vernier caliper. TMZ and TP3 each significantly reduced tumor volume (FIGS. 11A and 11B) and tumor weight (FIG. 11C).

### Example 17

### Synthesis of TP-RGD

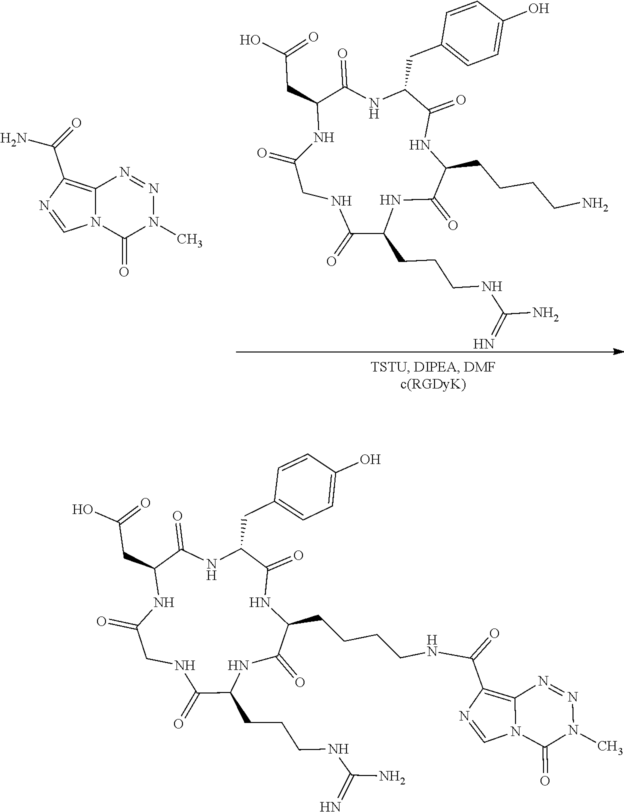 [SMILES: Cn1nnc2c(C(N)=O)ncn2c1=O ; N=C(N)NCCC[C@@H]1NC(=O)[C@H](CCCCN)NC(=O)[C@@H](Cc2ccc(O)cc2)NC(=O)[C@H](CC(=O)O)NC(=O)CNC1=O ; Cn1nnc2c(C(=O)NCCCC[C@@H]3NC(=O)[C@@H](Cc4ccc(O)cc4)NC(=O)[C@H](CC(=O)O)NC(=O)CNC(=O)[C@H](CCCNC(=N)N)NC3=O)ncn2c1=O]

To a mixture of temozolomide acid (24 mg, 0.12 mmol), and DIPEA (5 mL, 0.03 mmol) in DMF (6 ml) was added TSTU (36 mg, 0.12 mmol) and the reaction mixture was stirred at room temperature. After 2 hours of stirring, c(RGDyK) (75 mg, 0.12 mmol) in DMF was added. The reaction mixture was stirred under nitrogen overnight. DMF was removed in vacuo and the residue was purified using RP-HPLC.

### Example 18

### Stability of TP-RGD

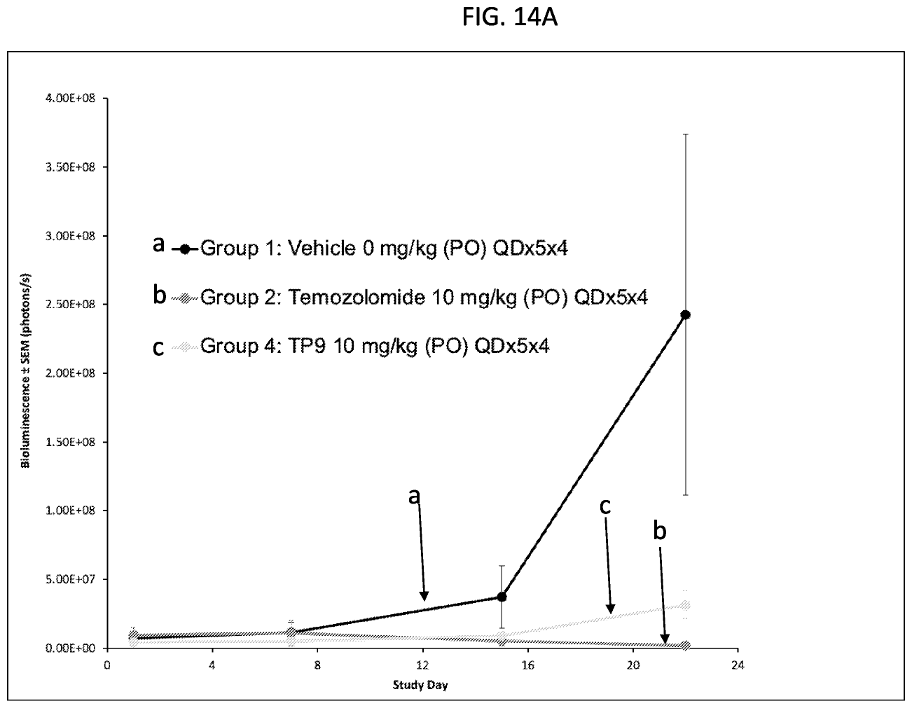

Hydrolytic chemical stability of TP-RGD was assessed (Table 12 and FIG. 13). 2 mg of TP-RGD was dissolved in 0.8 mL of phosphate buffer at pH>7.4. At room temperature there was no product formation. Then the reaction temperature was increased to 60° C. and incubated for 4 h. During the period of incubation, the reaction was monitored for every 30 min by LC-MS. Measure the AIC formation (% decomp) over the time.

TABLE 13
Hydrolytic chemical stability of TP-RGD

| Time (h) | % SM | % decomp. |
|---|---|---|
| 0.5 | 98..9 | 0 |
| 1 | 81.8 | 18.2 |
| 1.5 | 65.5 | 33.9 |
| 2 | 45.9 | 53.5 |
| 2.5 | 39.3 | 56.8 |
| 3 | 27.4 | 61.6 |
| 4 | 2.3 | 94.7 |
| 4.5 | 2.3 | 94.7 |

### Example 19

### Activity of TP-RGD

Activity of TP-RGD was assessed in glioma cell cultures, as described in Example 11. As shown in Table 14, TP-RGD had improved activity compared to TMZ.

TABLE 14
Effect of compounds on glioma cell viability

| | |
|---|---|
| MGMT | IC₅₀ (μmol/L), 72 h |

| Cell Line | Expression | TMZ | TP-RGD |
|---|---|---|---|
| U251 | − | 383 | 0.306 |
| A172 | − | >100 | 0.383 |
| U87MG | − | 440 | >100 |
| T98G | +++ | >600 | 0.254 |

The effect on U251 cell proliferation after 72 hours of treatment was also assessed using the BrdU ELISA cell proliferation assay as described in Example 11. The IC₅₀ for TMZ was >200 μM and was 70.2 μM for TP-RGD.

Expression of 53BP1 foci, a marker for double strand break formation, was analyzed in U251 cells as described in Example 11. Cells were treated with 10 μM of TMZ or 1 μM of TP-RGD for 48 hours and immuno-stained with antibodies to 53BP1. Rates of double-strand break repair were determined by scoring 53BP1 foci and plotting the average number of 53BP1 foci per cell (FIG. 13).

Permeability of the compounds was evaluated in the bidirectional Caco-2 assay as described in Example 11 (Table 15).

TABLE 15
Caco-2 permeability; P-gp efflux ratio

| Compound | Efflux Ratio |
|---|---|
| TMZ | 1.04 |
| TP-RGD | 0.57 |
| Propranolol | 1.27 |
| Vinblastine | 21.74 |

### Example 20

### Efficacy of TP9 in Mouse Model of GBM

Animal studies were performed in accordance with Translational Drug Development, LLC IACUC-approved protocols. Intracranial tumors were generated by injecting 3×10⁵ U87-Luc cells in 5 mL of growth media into the right corpus striatum (1 mm anterior and 2 mm lateral to the bregma, depth of 3 mm) of Athymic Nude (Crl:NU(NCr)-Foxn1nu) mice (Charles River, Hollister, CA). Treatment was initiated 7 days after injection. Mice with U87-Luc tumors were randomized to three treatment groups: control (Group 1, Vehicle, n=8), TMZ (Group 2, 10 mg/kg, n=8) and TP9 (Group 4, 10 mg/kg, n=8) on a schedule of daily for five days, with 2 days off, repeated for four cycles (QD×5×4). Oral gavage treatments were formulated in 10% PEG300 in PBS. Bioluminescence (Total ROI, photons/s) images were performed under anesthesia once weekly starting on Day 1 (7 days post inoculation of U87-Luc cells) and body weights were recorded twice weekly. Individual mice were euthanized at peri-morbidity, as defined by clinical signs suggesting high tumor burden. Upon peri-morbidity or at study end, mice were euthanized, brain tumor was excised from right side of brain, and a wet weight was recorded. If tumor was not grossly identifiable, expected tumor-region of the brain was collected. Tissue was fixed in formalin for 48-72 hours and then stored in 70% Ethanol.

A minimum of 100 μL/mouse of whole blood was collected via submandibular survival bleed on Day 10 and Day 27 from 3 mice per group (selected by TD2) from Groups 1, 2 and 4 for assessment of hematological toxicity. Blood was placed in K2-EDTA microtainer tubes (Becton, Dickinson & Co.; Franklin Lakes, NJ) and mixed thoroughly before CBC analysis at TD2.

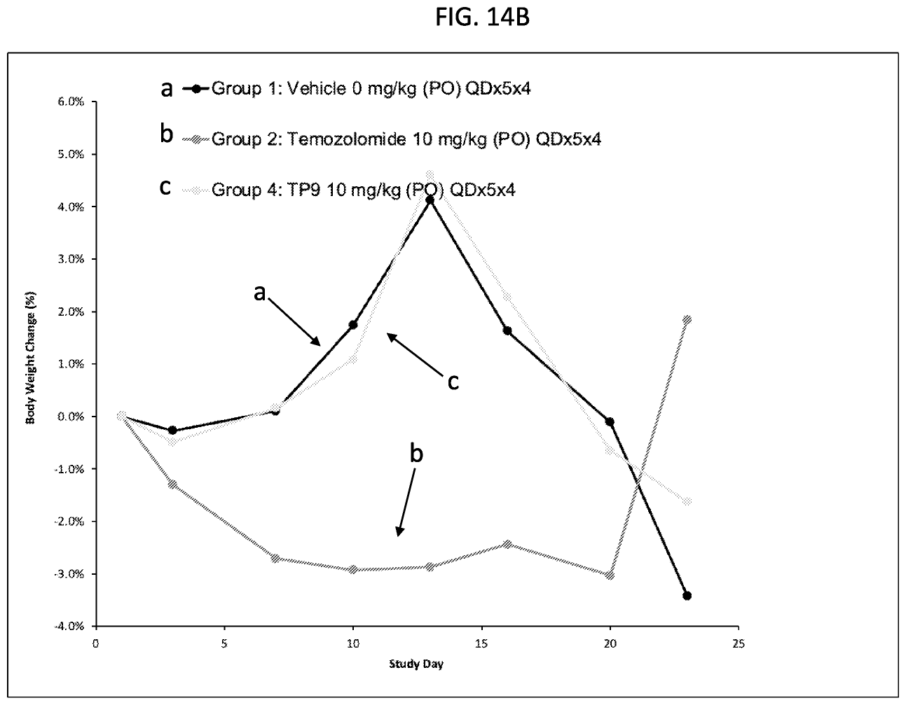

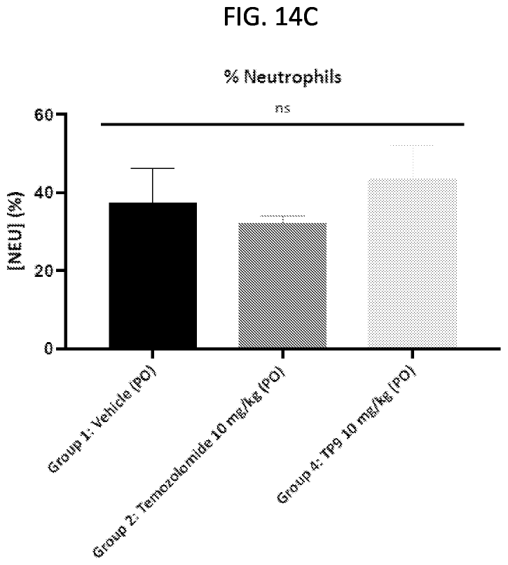

Treatment with both TMZ and TP9 reduced tumor burden (FIG. 14A). TMZ impacted body weight over the course of the study; however, TP9 did not affect body weight compared to control (FIG. 14B). There was not significant hematological toxicity, as assessed by percent neutrophils in whole blood (FIG. 14C).

In view of the many possible embodiments to which the principles of the present disclosure may be applied, it should be recognized that the illustrated embodiments are only preferred examples and should not be taken as limiting the scope of the present disclosure. Rather, the scope of the present disclosure is defined by the following claims. We therefore claim as our invention all that comes within the scope and spirit of these claims.

1. A compound having a structure according to Formula I, including any pharmaceutically acceptable salt, prodrug, hydrate, solvate, or tautomer thereof 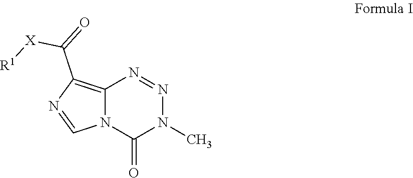
wherein
X is oxygen or NR², wherein R² is hydrogen, aliphatic, heteroaliphatic, haloaliphatic, haloheteroaliphatic, aromatic, or an organic functional group; and
R¹ is aliphatic, heteroaliphatic, haloaliphatic, haloheteroaliphatic, aromatic, an organic functional group, or a biologically active compound; provided that (i) if X is NR² and R² is hydrogen, then R¹ is not methyl, sec-butyl, iso-butyl, n-butyl, isopropyl, t-butyl, —(CH₂)₂-8-methyl-2,3,4,5-tetrahydro-1 H-pyrido[4,3-b]indole, —(CH₂)₂-2,8-dimethyl-2,3,4,5-tetrahydro-1 H-pyrido[4,3-b]indole, —(CH₂)₂-5,8-dimethyl-2,3,4,5-tetrahydro-1 H-pyrido[4,3-b]indole, or —(CH₂)₆—N(H)-aminobenzoic acid; and (ii) if X is O, and R¹ is an aliphatic group, then the aliphatic group is other than methyl, ethyl, n-propyl, n-butyl, n-hexyl, n-heptyl, n-octyl, or methyl substituted with an aromatic group.

2. The compound of claim 1, wherein R¹ is aliphatic, heteroaliphatic, aryl, or heteroaryl and wherein X is NR² wherein R² is hydrogen.

3. (canceled)

4. The compound of claim 1, wherein the compound has a structure according to Formula II, including any pharmaceutically acceptable salt, prodrug, hydrate, solvate, or tautomer thereof 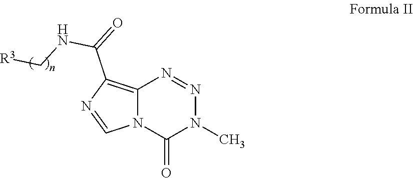
wherein R³ is CH₃, ester, amide, alkoxy, or amine; and n is an integer ranging from 1 to 10.

5. The compound of claim 4, wherein R³ is CH₃, —OC(O)aliphatic, —N(H)C(O)aliphatic, —O-aliphatic, —N(aliphatic)₂, or -NHaliphatic; and n is an integer selected from 1 to 5.

6. The compound of claim 4, wherein R³ is CH₃, —OC(O)Me, —N(H)C(O)Me, -OtBu, —N(Me)₂, or -NHEt; and n is 5.

7. The compound of claim 1, wherein the compound has a structure according to Formula III, including any pharmaceutically acceptable salt, prodrug, hydrate, solvate, or tautomer thereof 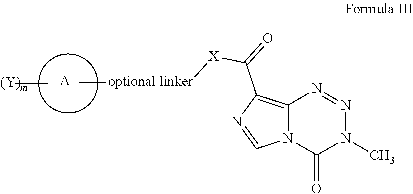
wherein ring A is an aliphatic ring system, a heteroaliphatic ring system, or an aromatic ring system; the optional linker is present or not and, if present, is an aliphatic group; each Y independently is halogen, alkyl, haloalkyl, ester, or sulfonamide; and m is an integer selected from 0 to 10.

8. The compound of claim 7, wherein ring A is a three-, four-, five-, six-, seven-, eight-, nine-, ten-, eleven-, or twelve-membered aliphatic ring system.

9. The compound of claim 7, wherein the aliphatic ring system is cyclobutyl, cyclopentyl, cyclohexyl, bicyclopentyl, bicyclohexyl, bicycloheptenyl, or adamantyl.

10. The compound of claim 7, wherein each Y independently is Cl, F, Br, I, CH₃, CF₃, —OC(O)CH₃, or —SO₂NH₂.

11. The compound of claim 7, wherein m is 0, 1, or 2.

12. The compound of claim 7, wherein the compound has a structure according to Formula IIIA′, including any pharmaceutically acceptable salt, prodrug, hydrate, solvate, or tautomer thereof, 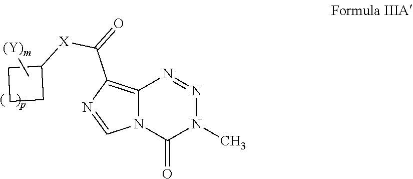
wherein p is an integer selected from 0 to 5.

13. The compound of claim 7, wherein the compound has a structure according to Formula IIIA″, including any pharmaceutically acceptable salt, prodrug, hydrate, solvate, or tautomer thereof, 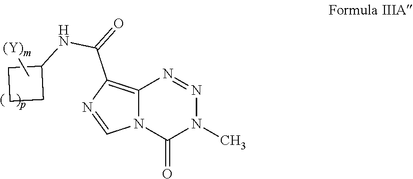
wherein p is an integer selected from 0 to 5.

14. The compound of claim 7, wherein the compound has a structure according to Formula IIIB, including any pharmaceutically acceptable salt, prodrug, hydrate, solvate, or tautomer thereof, 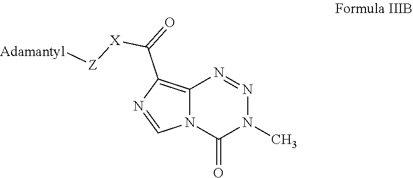
wherein X is oxygen or NH; and Z is bond or an aliphatic group the aliphatic group comprises one or more methylene groups, one or more cyclic groups, or a combination thereof and wherein the cyclic group is bound to the adamantyl or forms a spirocyclic group with the adamantyl.

15. (canceled)

16. The compound of claim 7, wherein the compound has a structure according to Formula IIIC, including any pharmaceutically acceptable salt, prodrug, hydrate, solvate, or tautomer thereof, 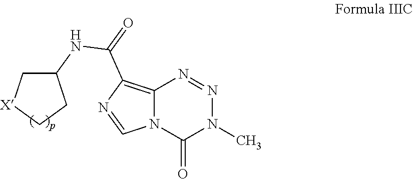
wherein X′ is a selected from O, NH, or S and p is an integer selected from 0 to 5.

17. The compound of claim 16, wherein the compound has a structure according to Formula IIIC′ or IIIC″, including any pharmaceutically acceptable salt, prodrug, hydrate, solvate, or tautomer thereof, 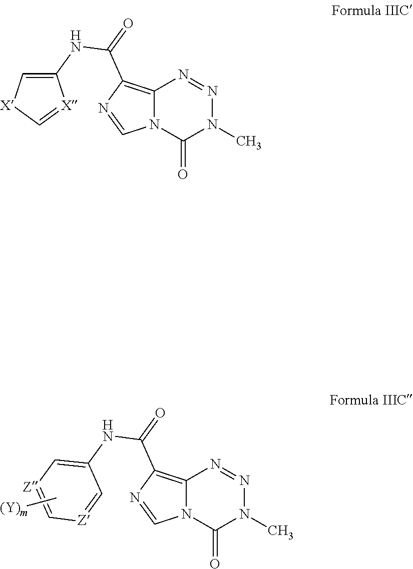
wherein X′ is O, NH, S; X″ is selected from O, N, S, or CH; and each of Z′ and Z″ independently is CH, N, or an oxidized N atom.

18. The compound of claim 7, wherein the compound has a structure according to Formulas IIID′ or IIID″, including any pharmaceutically acceptable salt, prodrug, hydrate, solvate, or tautomer thereof.

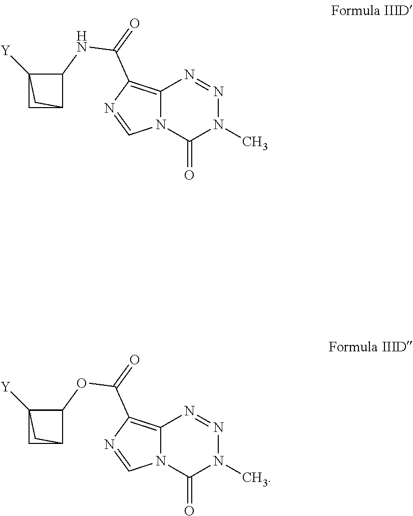

19. The compound of claim 1, wherein the compound is selected from

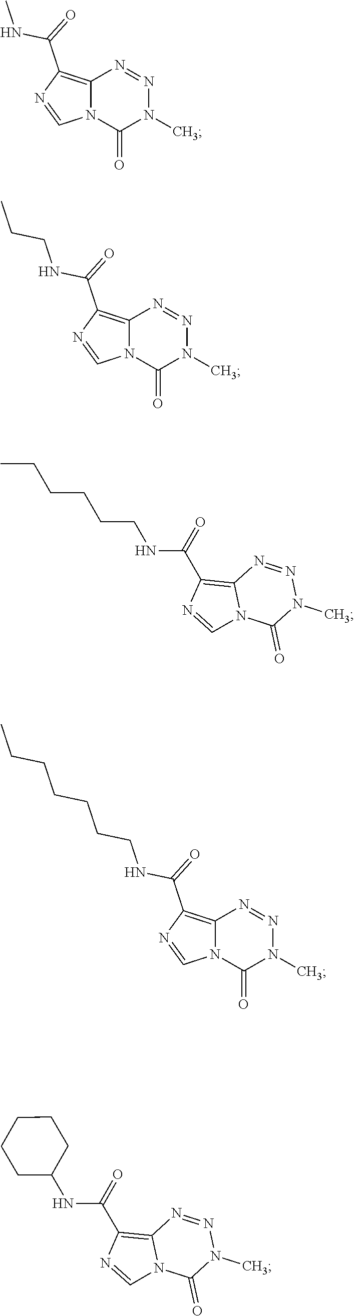 [SMILES: CNC(=O)c1ncn2c(=O)n(C)nnc12 ; CCCNC(=O)c1ncn2c(=O)n(C)nnc12 ; CCCCCCNC(=O)c1ncn2c(=O)n(C)nnc12 ; CCCCCCCNC(=O)c1ncn2c(=O)n(C)nnc12 ; Cn1nnc2c(C(=O)NC3CCCCC3)ncn2c1=O]

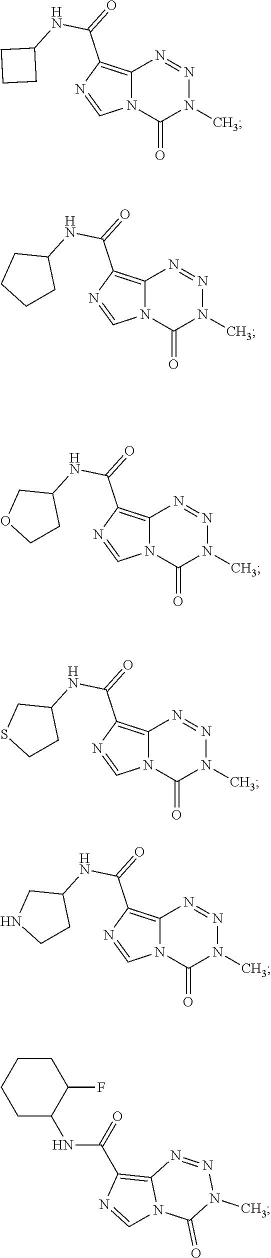 [SMILES: Cn1nnc2c(C(=O)NC3CCC3)ncn2c1=O ; Cn1nnc2c(C(=O)NC3CCCC3)ncn2c1=O ; Cn1nnc2c(C(=O)NC3CCOC3)ncn2c1=O ; Cn1nnc2c(C(=O)NC3CCSC3)ncn2c1=O ; Cn1nnc2c(C(=O)NC3CCNC3)ncn2c1=O ; Cn1nnc2c(C(=O)NC3CCCCC3F)ncn2c1=O]

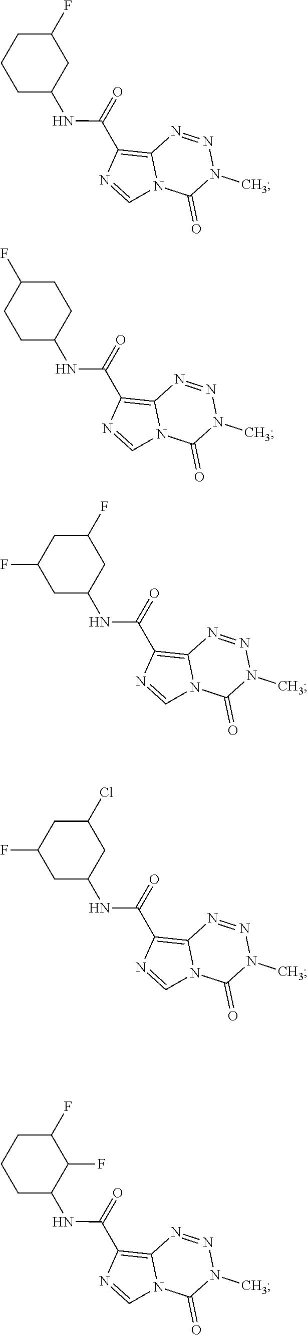 [SMILES: Cn1nnc2c(C(=O)NC3CCCC(F)C3)ncn2c1=O ; Cn1nnc2c(C(=O)NC3CCC(F)CC3)ncn2c1=O ; Cn1nnc2c(C(=O)NC3CC(F)CC(F)C3)ncn2c1=O ; Cn1nnc2c(C(=O)NC3CC(F)CC(Cl)C3)ncn2c1=O ; Cn1nnc2c(C(=O)NC3CCCC(F)C3F)ncn2c1=O]

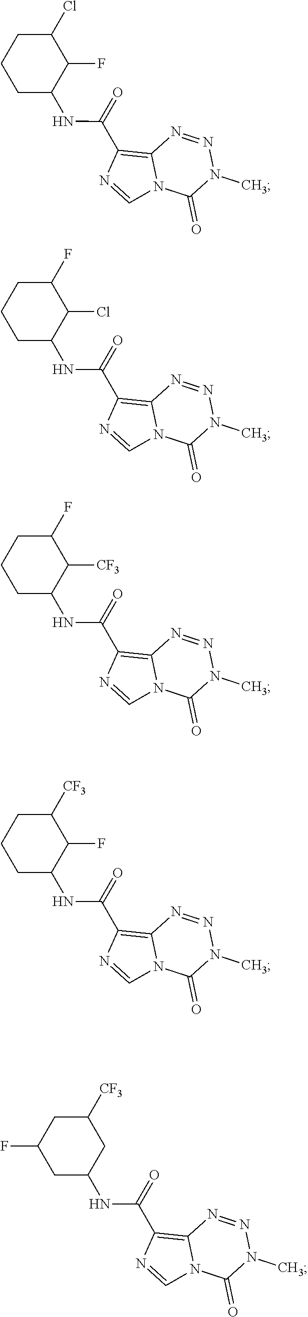 [SMILES: Cn1nnc2c(C(=O)NC3CCCC(Cl)C3F)ncn2c1=O ; Cn1nnc2c(C(=O)NC3CCCC(F)C3Cl)ncn2c1=O ; Cn1nnc2c(C(=O)NC3CCCC(F)C3C(F)(F)F)ncn2c1=O ; Cn1nnc2c(C(=O)NC3CCCC(C(F)(F)F)C3F)ncn2c1=O ; Cn1nnc2c(C(=O)NC3CC(F)CC(C(F)(F)F)C3)ncn2c1=O]

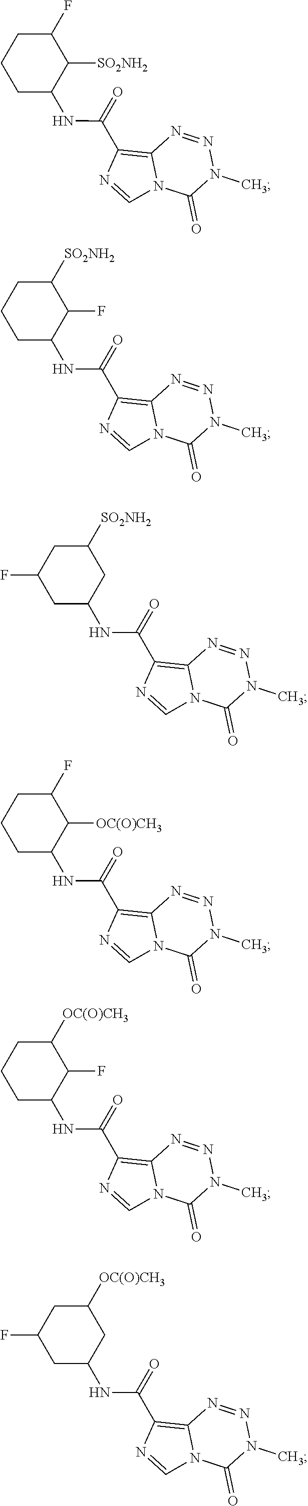

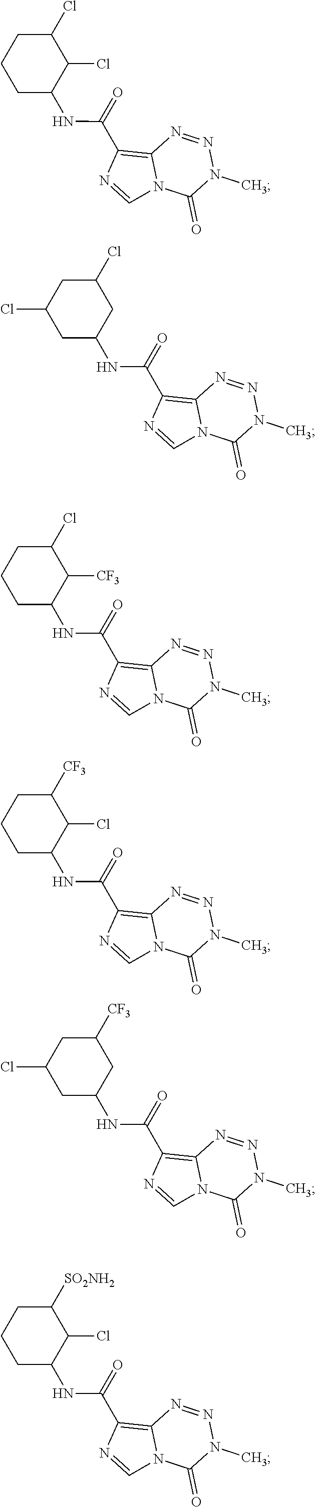 [SMILES: Cn1nnc2c(C(=O)NC3CCCC(Cl)C3Cl)ncn2c1=O ; Cn1nnc2c(C(=O)NC3CC(Cl)CC(Cl)C3)ncn2c1=O ; Cn1nnc2c(C(=O)NC3CCCC(Cl)C3C(F)(F)F)ncn2c1=O ; Cn1nnc2c(C(=O)NC3CCCC(C(F)(F)F)C3Cl)ncn2c1=O ; Cn1nnc2c(C(=O)NC3CC(Cl)CC(C(F)(F)F)C3)ncn2c1=O ; Cn1nnc2c(C(=O)NC3CCCC(S(N)(=O)=O)C3Cl)ncn2c1=O]

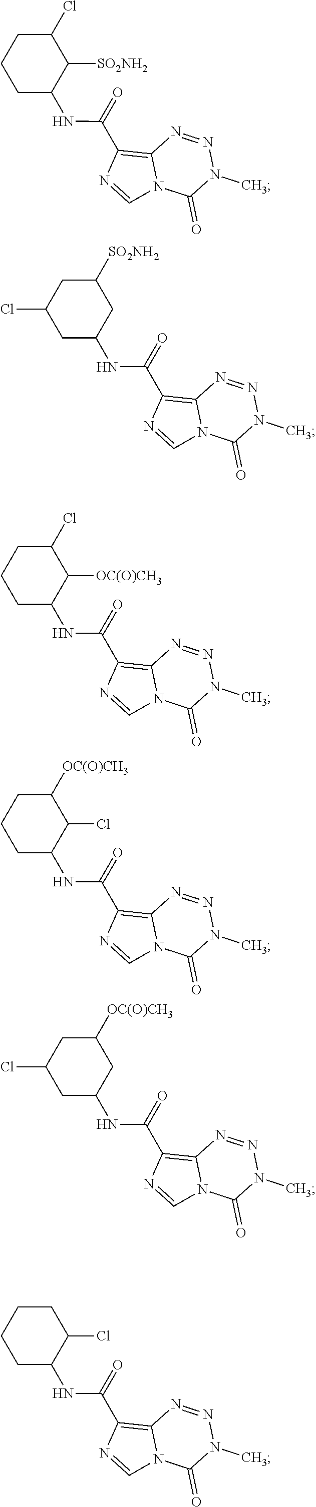

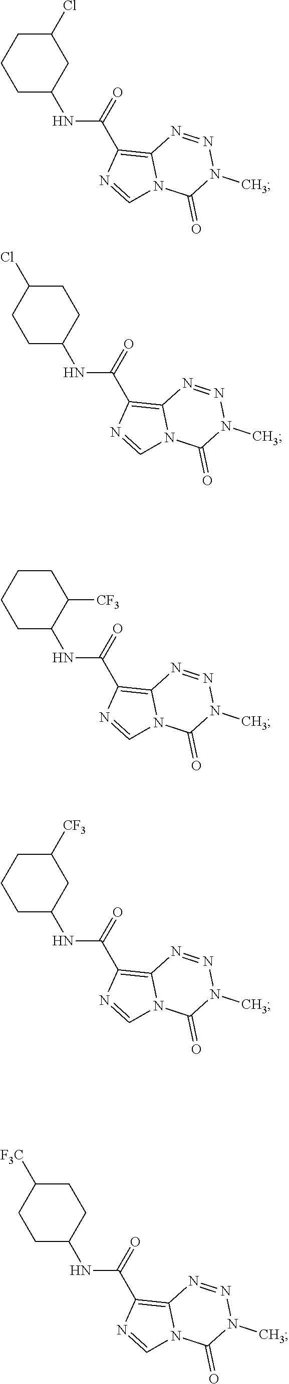 [SMILES: Cn1nnc2c(C(=O)NC3CCCC(Cl)C3)ncn2c1=O ; Cn1nnc2c(C(=O)NC3CCC(Cl)CC3)ncn2c1=O ; Cn1nnc2c(C(=O)NC3CCCCC3C(F)(F)F)ncn2c1=O ; Cn1nnc2c(C(=O)NC3CCCC(C(F)(F)F)C3)ncn2c1=O ; Cn1nnc2c(C(=O)NC3CCC(C(F)(F)F)CC3)ncn2c1=O]

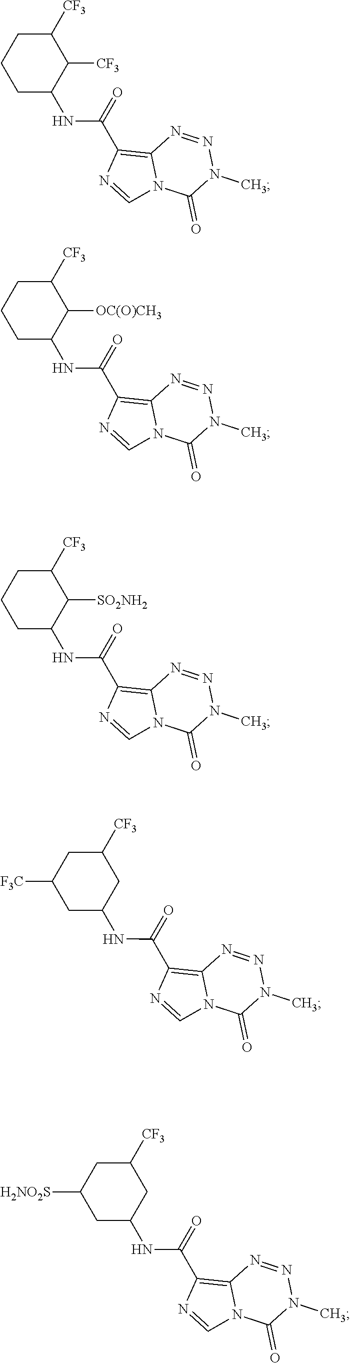

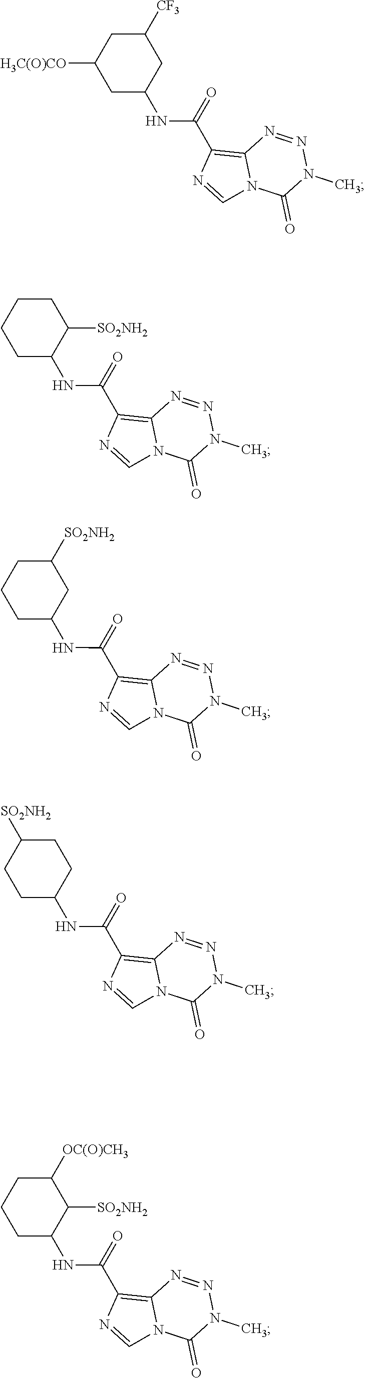

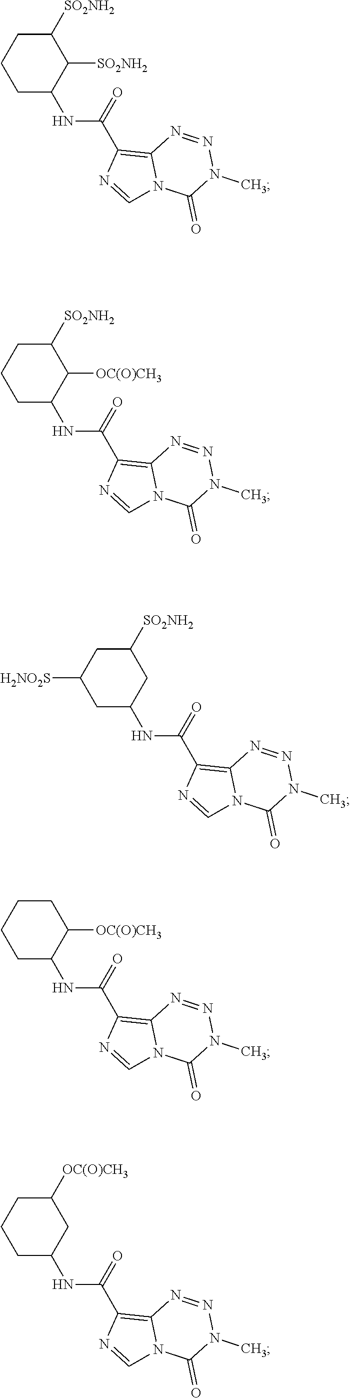

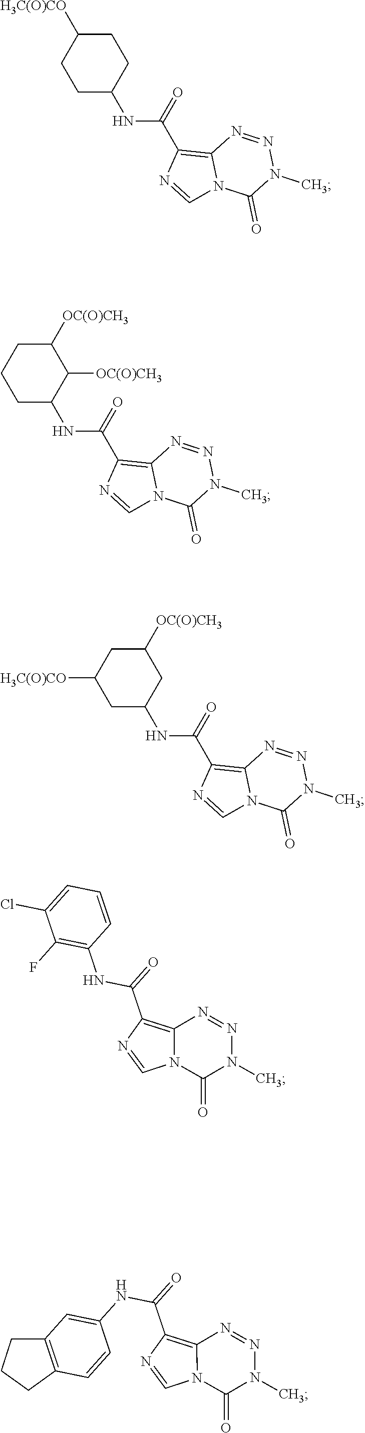

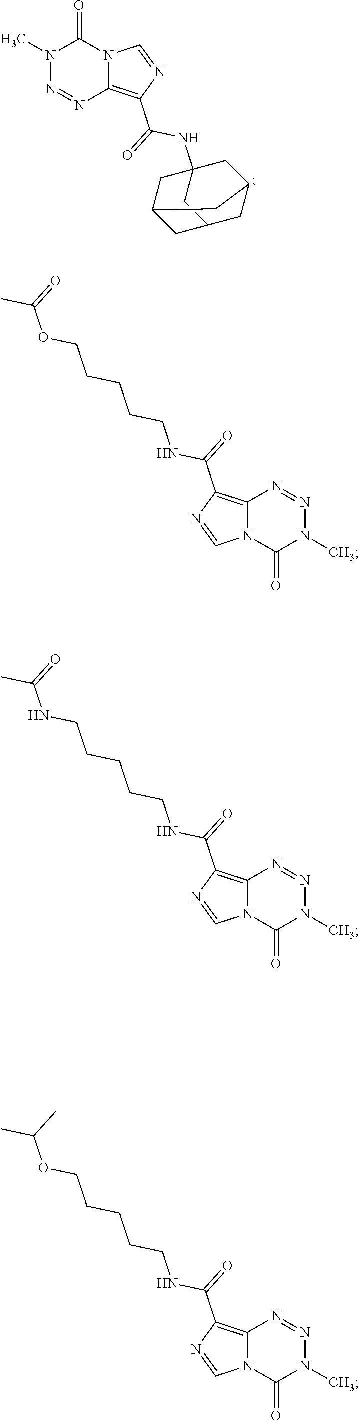 [SMILES: Cn1nnc2c(C(=O)NC34CC5CC(CC(C5)C3)C4)ncn2c1=O ; CC(=O)OCCCCCNC(=O)c1ncn2c(=O)n(C)nnc12 ; CC(=O)NCCCCCNC(=O)c1ncn2c(=O)n(C)nnc12 ; CC(C)OCCCCCNC(=O)c1ncn2c(=O)n(C)nnc12]

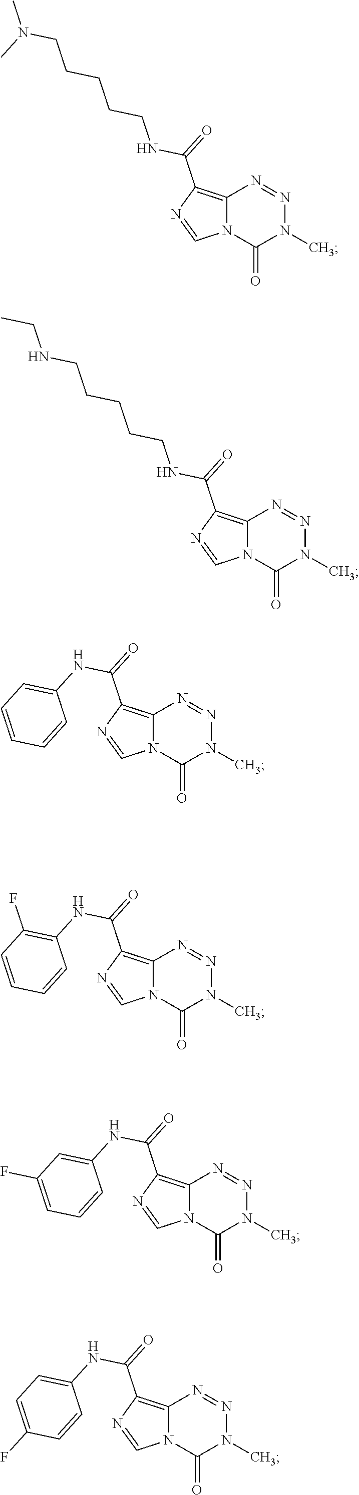 [SMILES: CN(C)CCCCCNC(=O)c1ncn2c(=O)n(C)nnc12 ; CCNCCCCCNC(=O)c1ncn2c(=O)n(C)nnc12 ; Cn1nnc2c(C(=O)Nc3ccccc3)ncn2c1=O ; Cn1nnc2c(C(=O)Nc3ccccc3F)ncn2c1=O ; Cn1nnc2c(C(=O)Nc3cccc(F)c3)ncn2c1=O ; Cn1nnc2c(C(=O)Nc3ccc(F)cc3)ncn2c1=O]

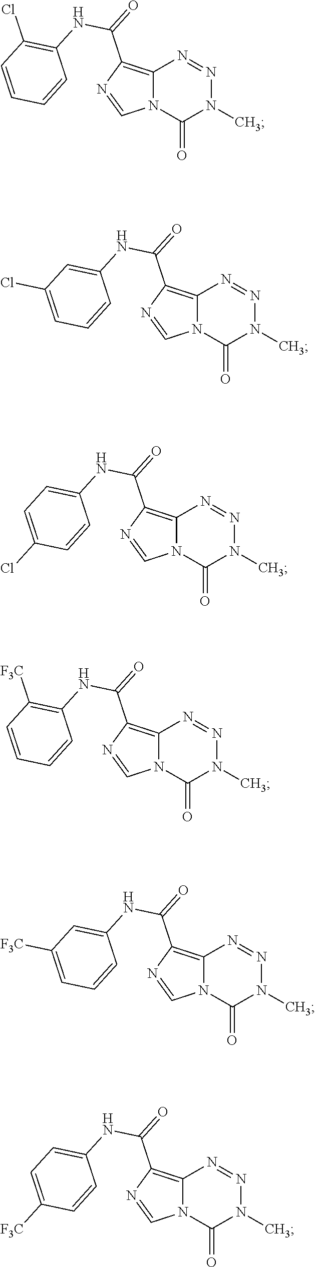 [SMILES: Cn1nnc2c(C(=O)Nc3ccccc3Cl)ncn2c1=O ; Cn1nnc2c(C(=O)Nc3cccc(Cl)c3)ncn2c1=O ; Cn1nnc2c(C(=O)Nc3ccc(Cl)cc3)ncn2c1=O ; Cn1nnc2c(C(=O)Nc3ccccc3C(F)(F)F)ncn2c1=O ; Cn1nnc2c(C(=O)Nc3cccc(C(F)(F)F)c3)ncn2c1=O ; Cn1nnc2c(C(=O)Nc3ccc(C(F)(F)F)cc3)ncn2c1=O]

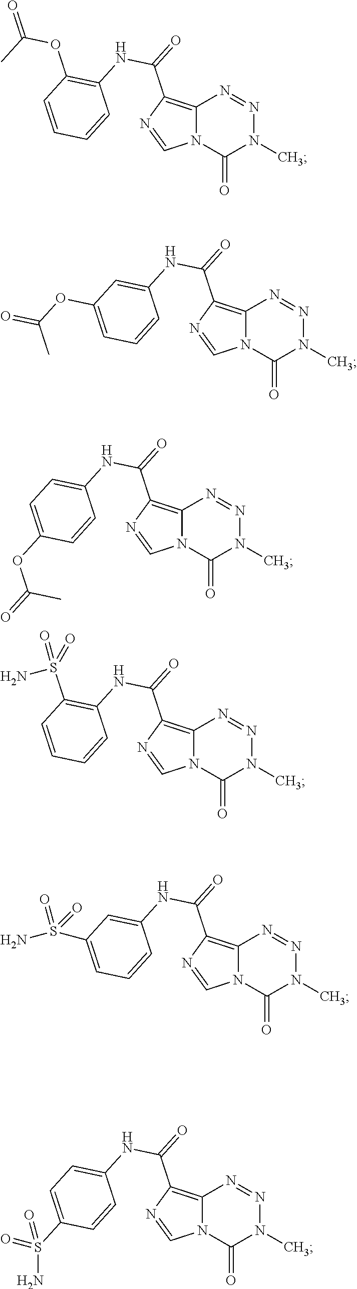

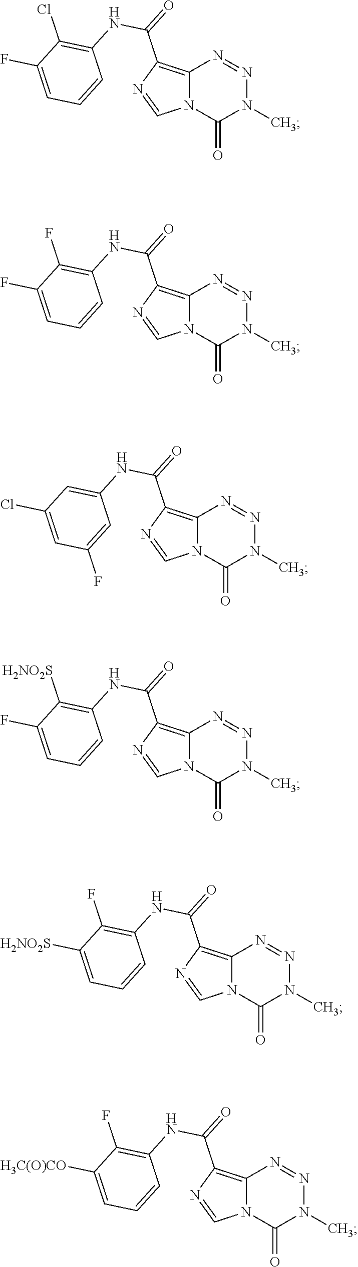

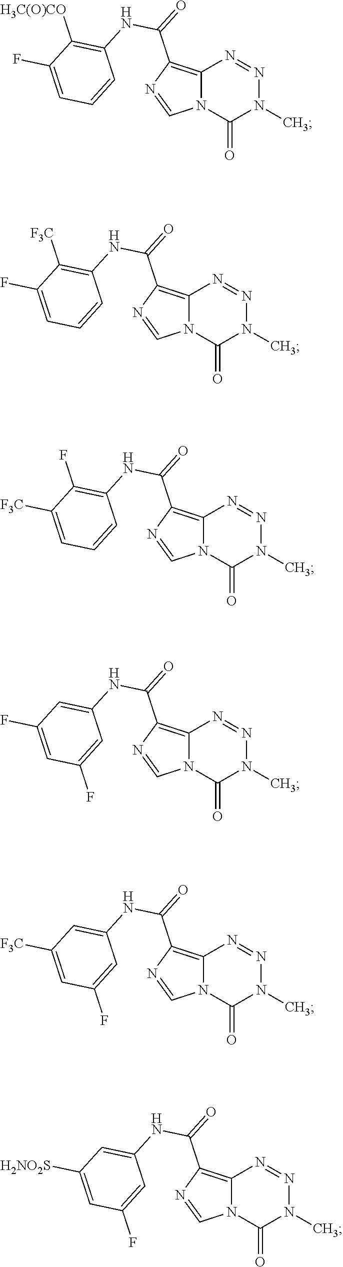

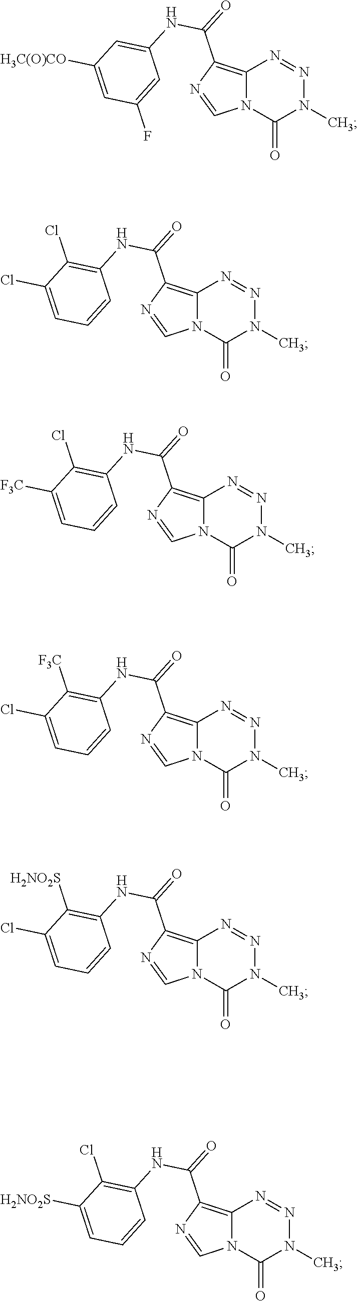

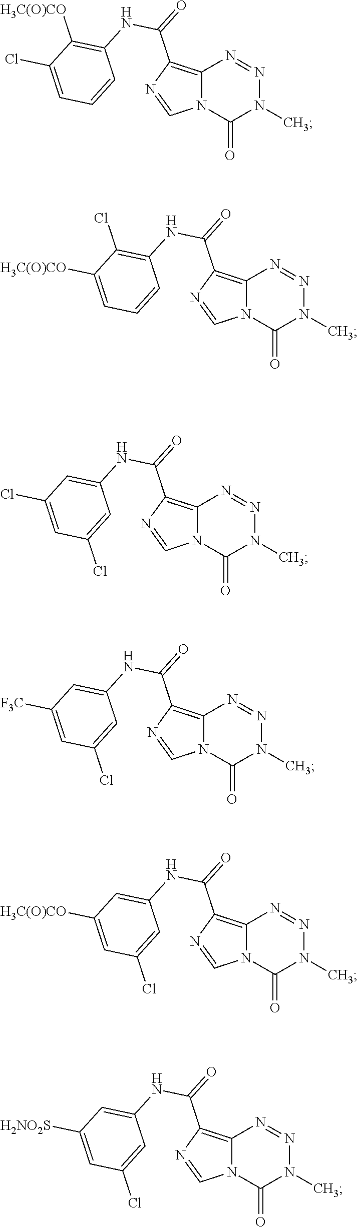

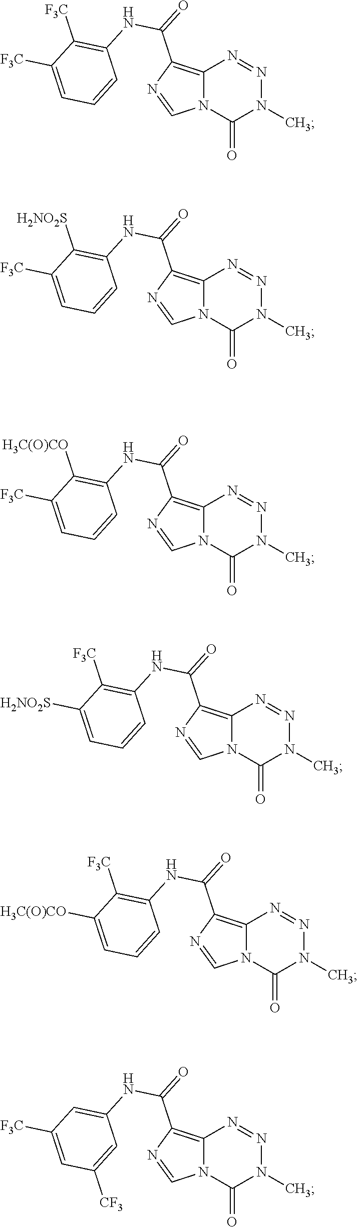

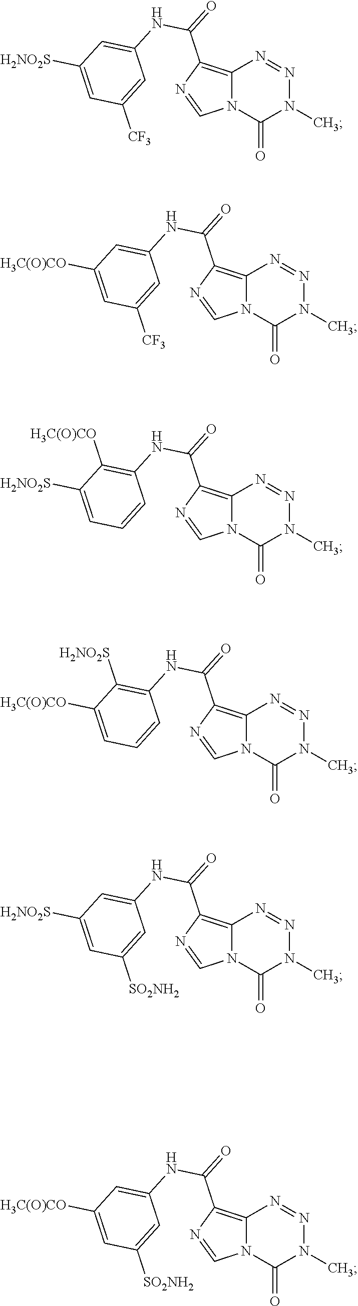

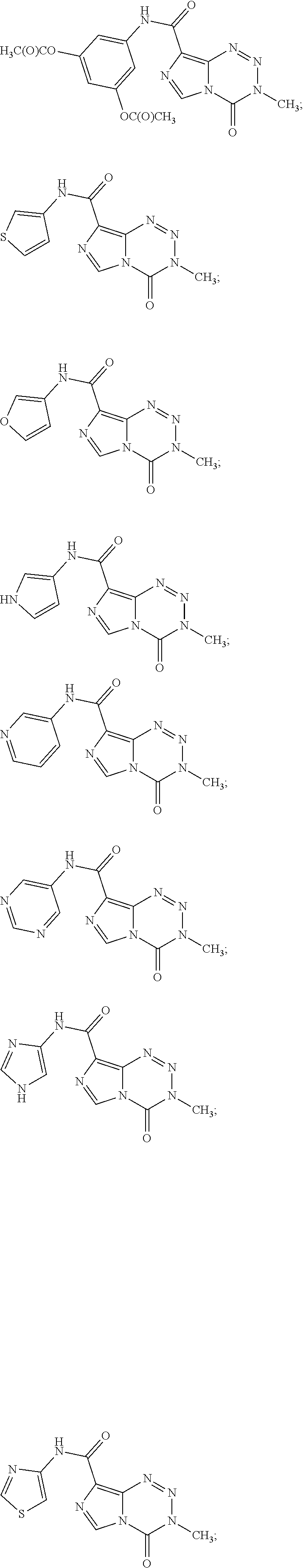

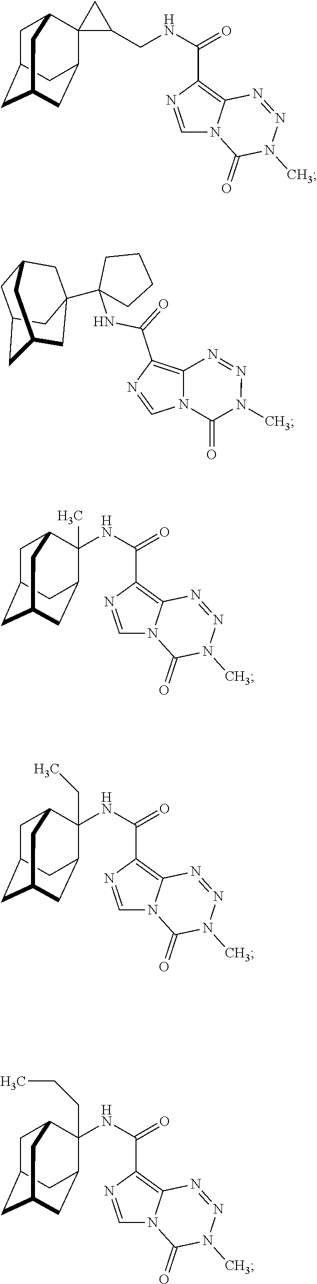

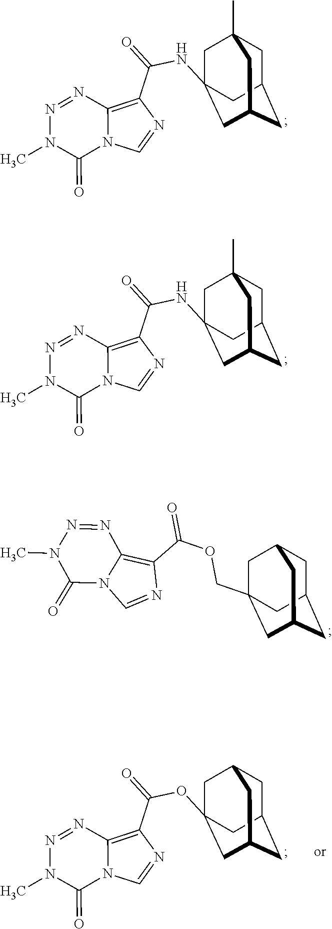

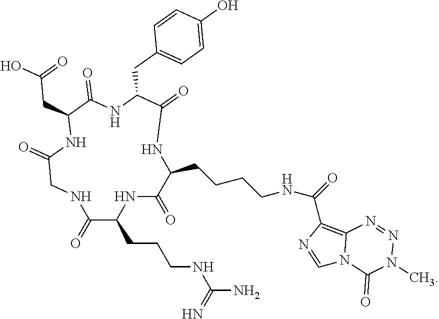 [SMILES: Cn1nnc2c(C(=O)NCCCC[C@@H]3NC(=O)[C@@H](Cc4ccc(O)cc4)NC(=O)[C@H](CC(=O)O)NC(=O)CNC(=O)[C@H](CCCNC(=N)N)NC3=O)ncn2c1=O]

20. The compound of claim 1, wherein the compound is selected from

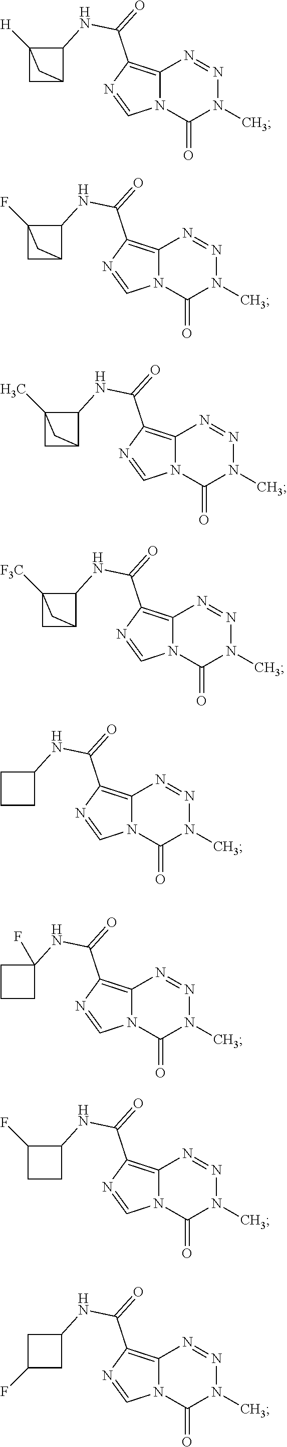

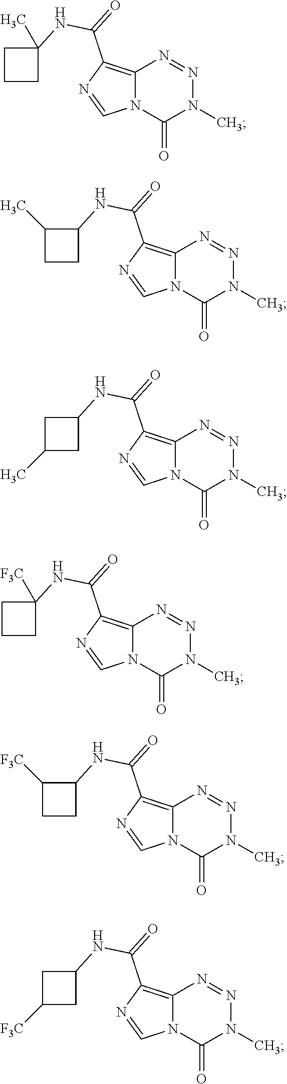

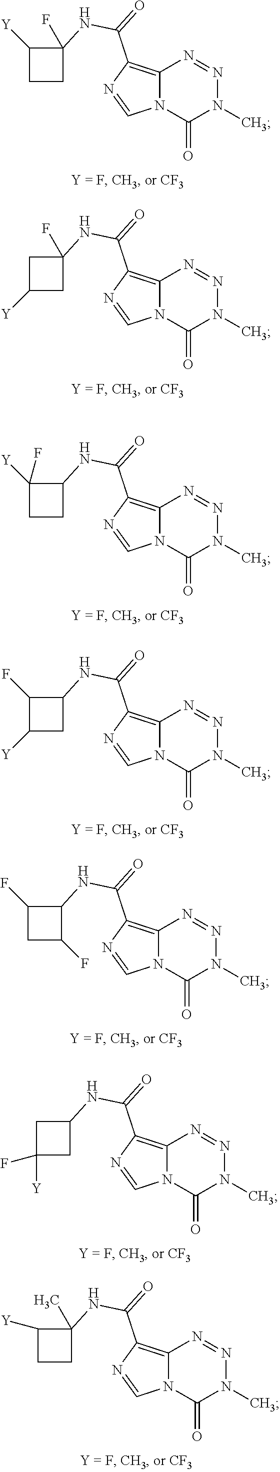

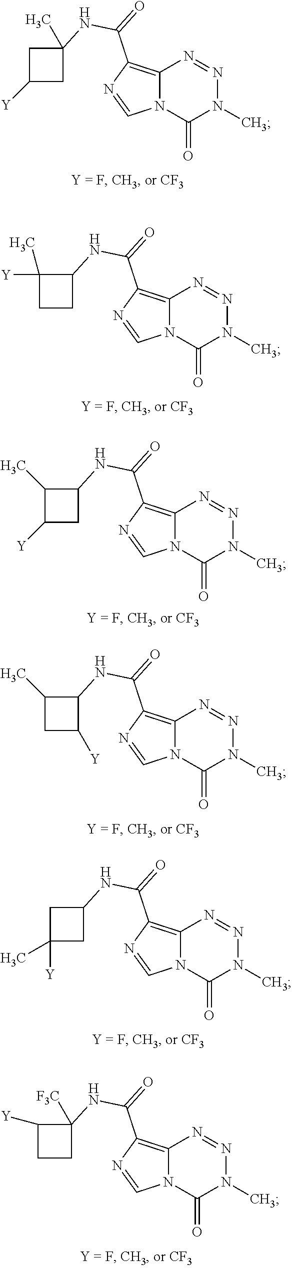

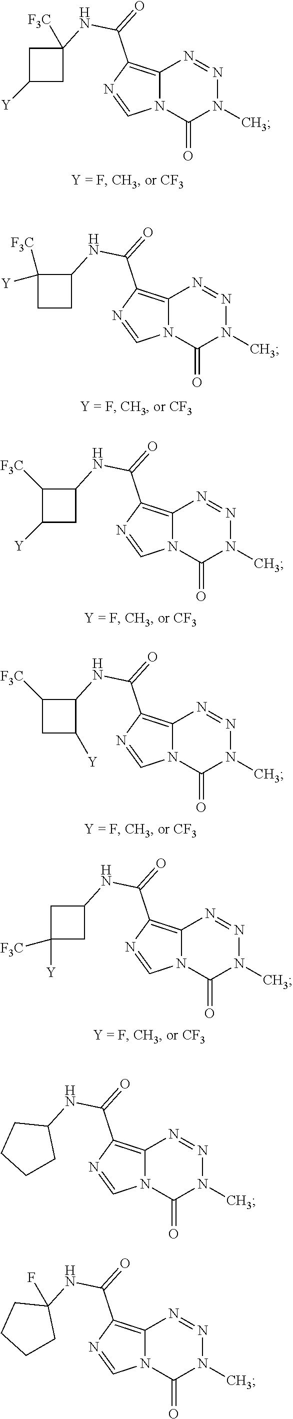

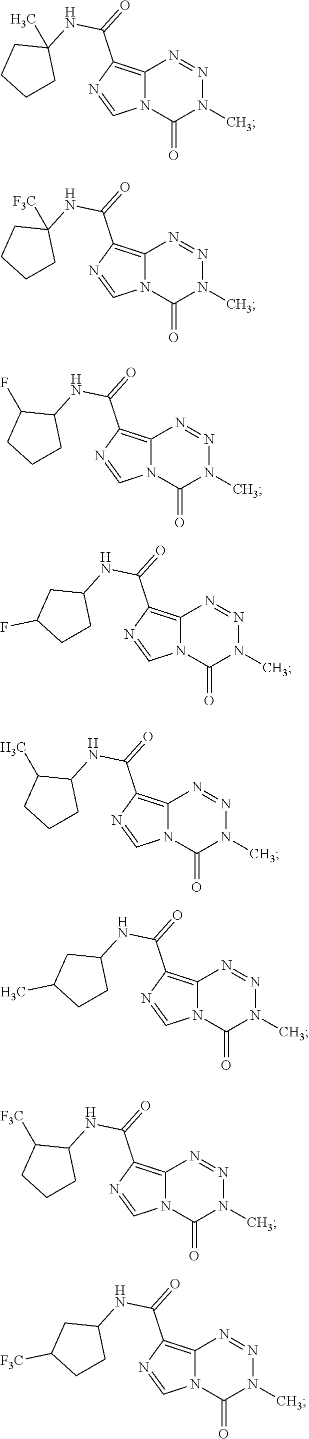

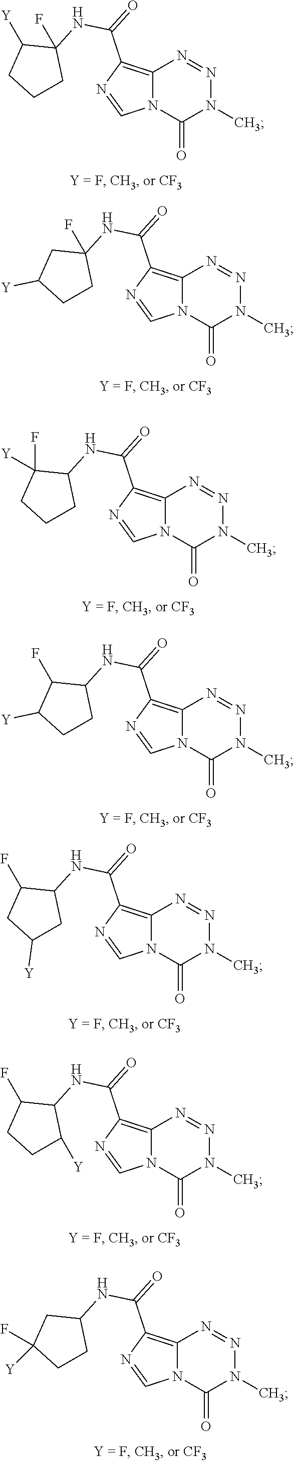

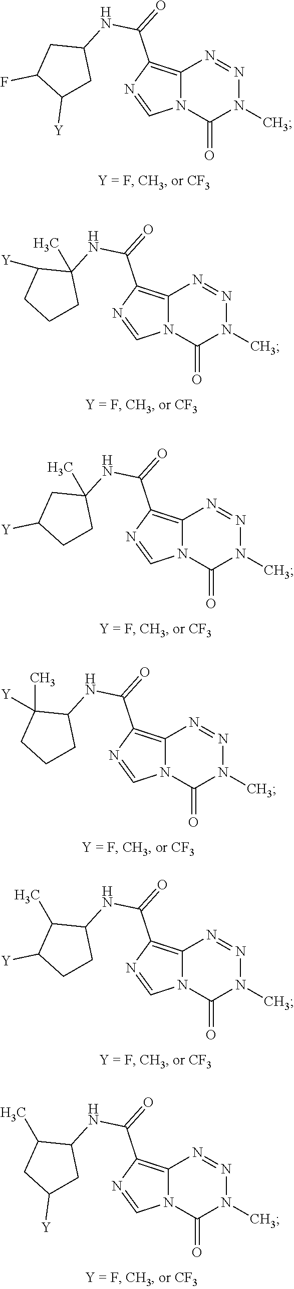

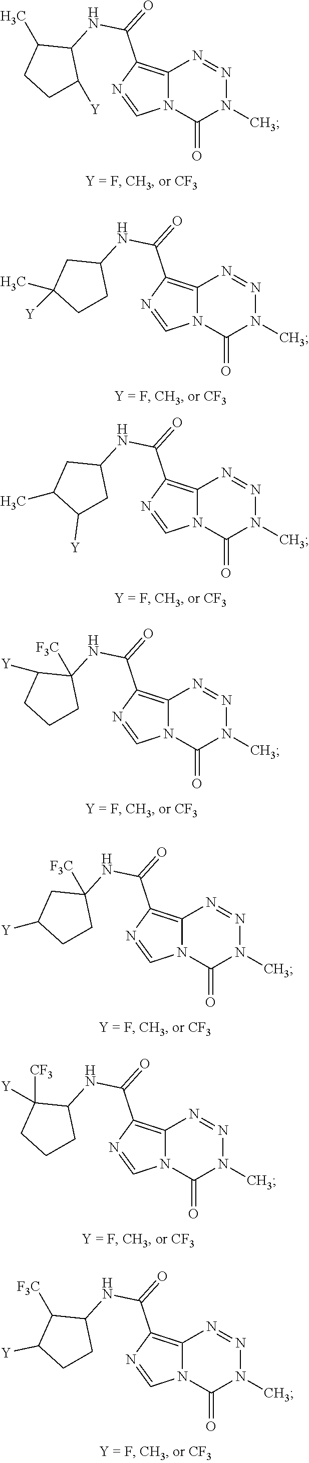

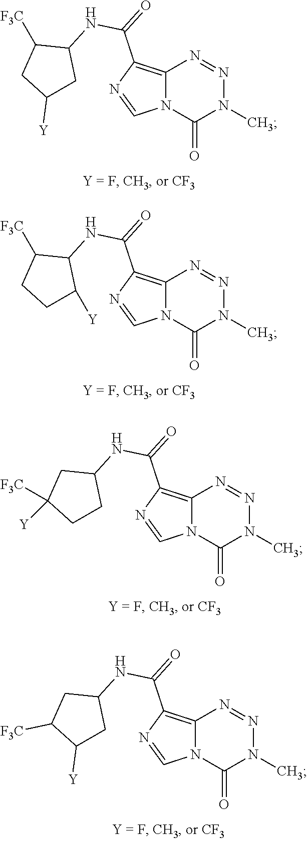

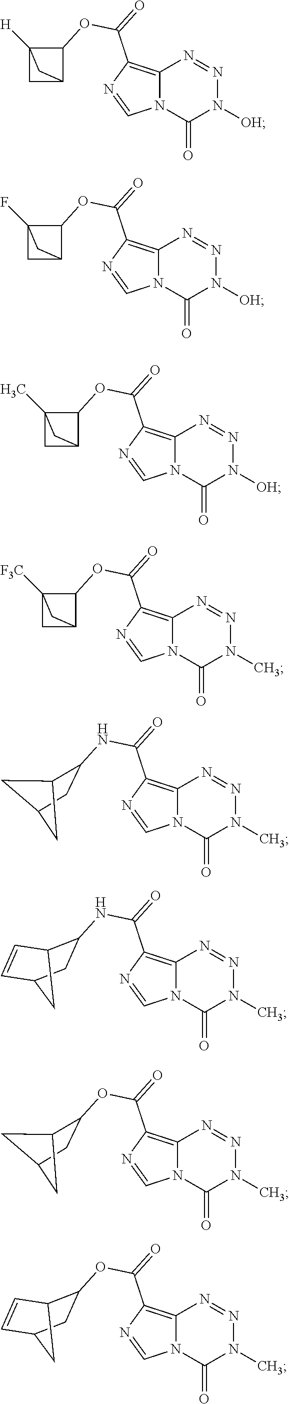

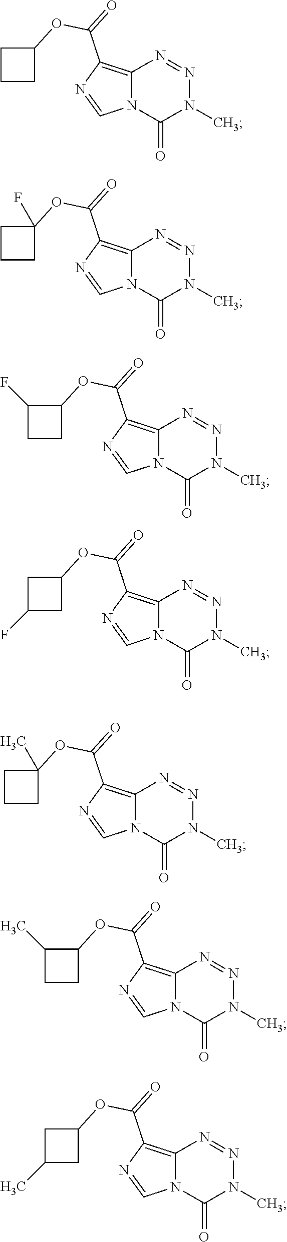

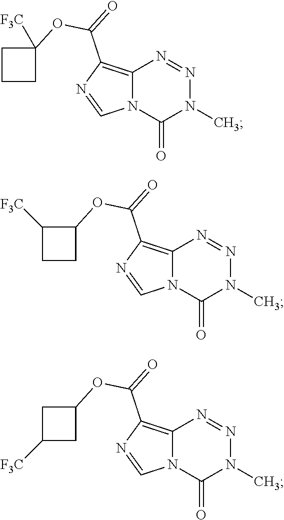

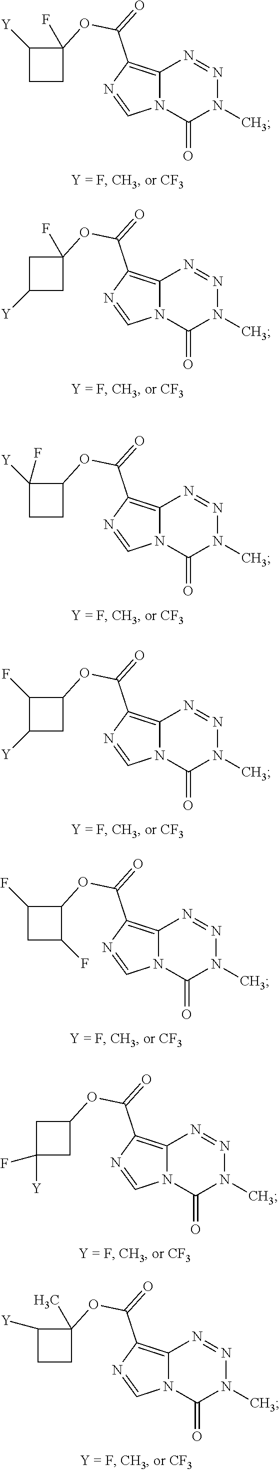

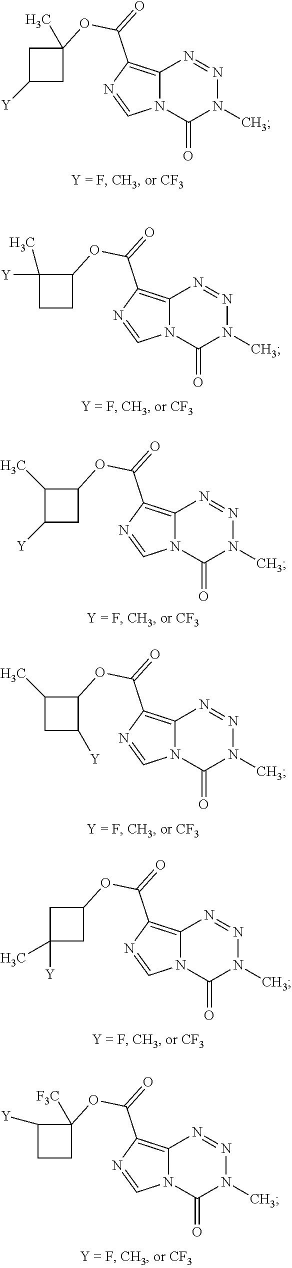

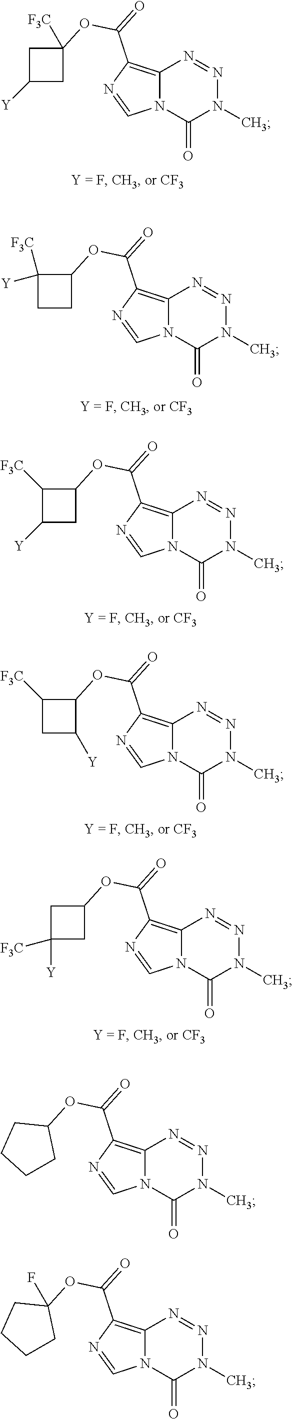

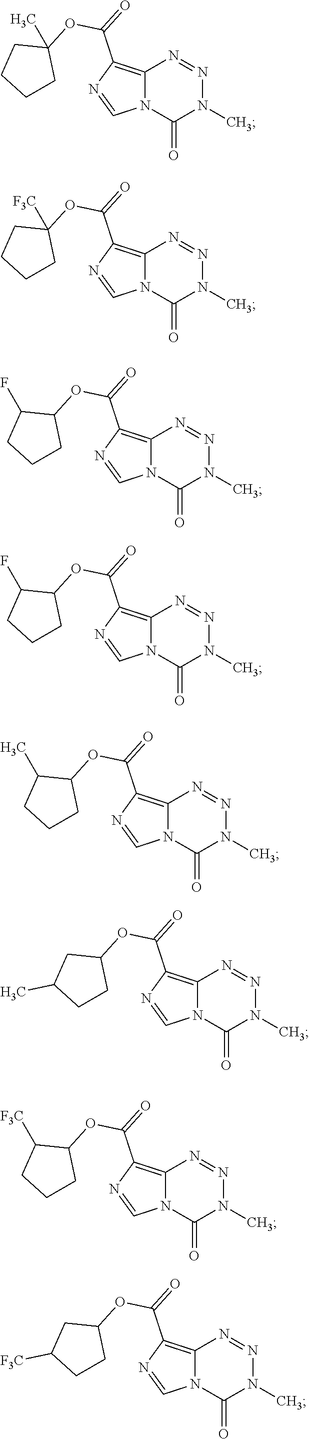

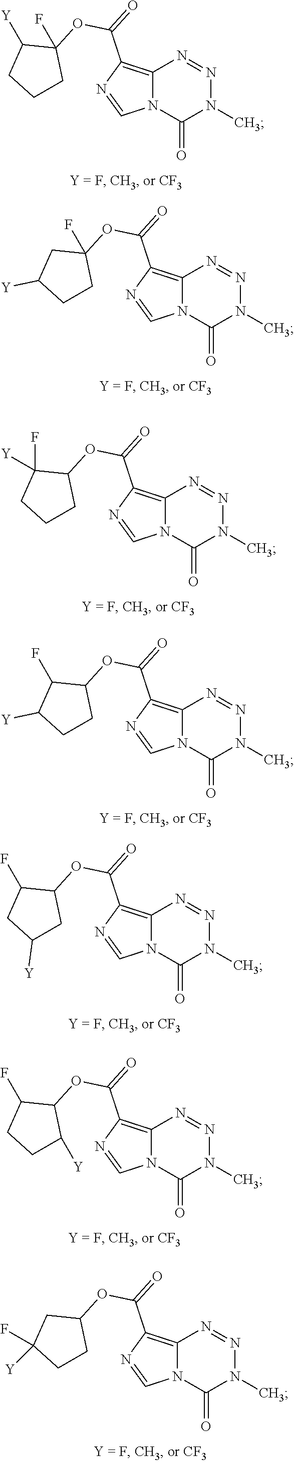

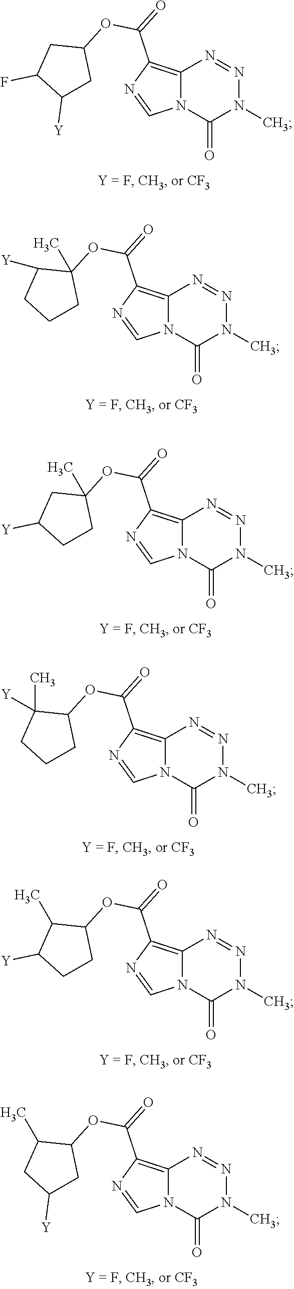

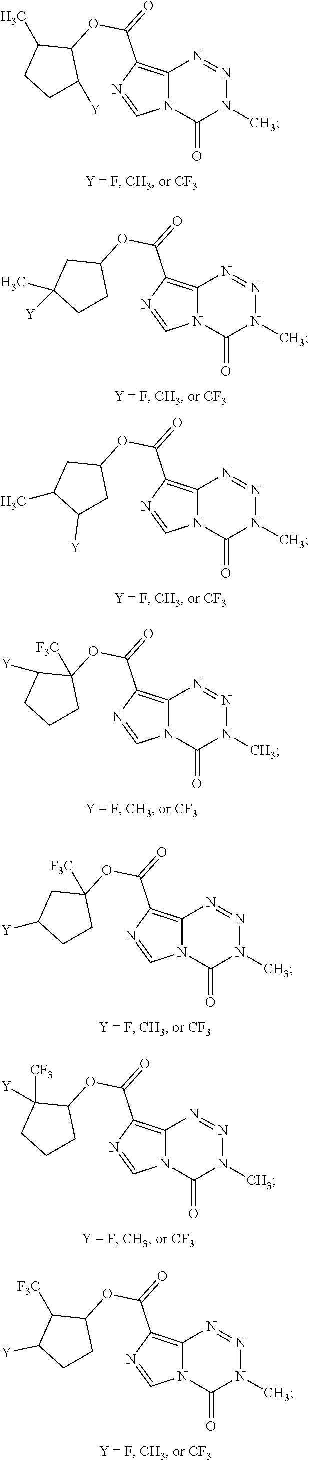

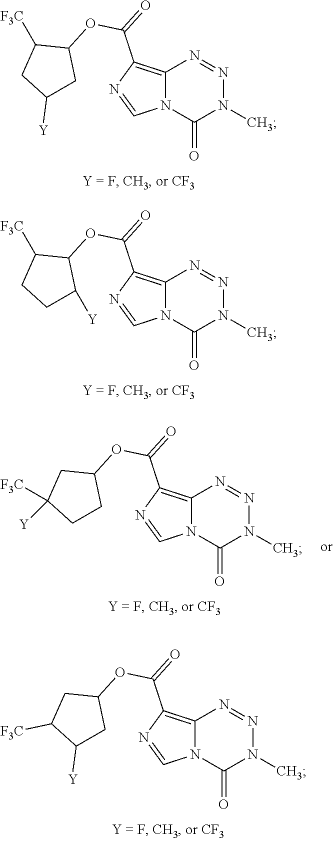

21. A composition comprising the compound of claim 1 and a pharmaceutically acceptable carrier.

22. A method of treating a subject with cancer, comprising administering to the subject an effective amount of the compound of claim 1, or a pharmaceutical composition thereof, to the subject.

23. The method of claim 22, wherein the subject with cancer has glioblastoma, breast cancer, gastric cancer, colorectal cancer, head and neck cancer, melanoma, lung cancer, pancreatic cancer, bladder cancer, prostate cancer, or acute myeloblastic leukemia.

24. The method of claim 23, wherein the subject with cancer has glioblastoma multiforme or a tumor that expresses or overexpresses O⁶-methylguanine DNA methyltransferase (MGMT) and/or has hypomethylation of MGMT promoter.

25-26. (canceled)

27. The method of claim 22, further comprising administering to the subject one of more of surgery, radiation therapy, chemotherapy, or immunotherapy.
